(19) United States
(12) Patent Application Publication
(10) Pub. No.: US 2024/0285740 A1
(43) Pub. Date: Aug. 29, 2024

(54) COMPOSITIONS AND METHODS FOR TREATING CANCER

(71) Applicant: Dana-Farber Cancer Institute, Inc., Boston, MA (US)

(72) Inventors: Kenneth C. Anderson, Wellesley, MA (US); Jooeun Bae, West Roxbury, MA (US)

(73) Assignee: Dana-Farber Cancer Institute, Inc., Boston, MA (US)

(21) Appl. No.: 18/289,780

(22) PCT Filed: May 9, 2022
(86) PCT No.: PCT/US2022/028317

§ 371 Date: Nov. 7, 2023

Related U.S. Application Data
(60) Provisional application No. 63/187,538, filed on May 12, 2021.

(51) Int. Cl. A61K 39/00; A61K 9/51; A61K 38/17; A61K 39/395; A61K 45/06; A61P 35/00

(52) U.S. Cl. CPC A61K 39/001152; A61K 9/51; A61K 38/1774; A61K 39/001111; A61K 39/001129; A61K 39/3955; A61K 39/4611; A61K 45/06; A61P 35/00; A61K 2039/54; A61K 2039/542; A61K 2039/543

(57) ABSTRACT

This disclosure features human Lymphocyte-activation gene 3 (LAG3) and human Galectin-3 (GAL3) inhibitory agents, immunogenic peptides X-Box Binding Protein 1 (XBP1), CD2 Subset 1 (CS1), and CD138 peptides, and methods of using the inhibitory agents and peptides to treat blood cancers and pre-cancerous conditions.

### CROSS-REFERENCE TO RELATED APPLICATION

This application claims the benefit of priority of U.S. Provisional Appl. No. 63/187,538, filed May 12, 2021, the content of which is incorporated herein by reference in its entirety.

### STATEMENT OF FEDERALLY SPONSORED RESEARCH

This invention was made with government support under Grant Nos. RO1-207237, RO1-124929, P50-100007 and PO1-155258 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Apr. 21, 2022, is named 00530-0412WO1_SL.txt and is 47,441 bytes in size.

### TECHNICAL FIELD

This disclosure relates to compositions and methods for the treatment of cancer (e.g., blood cancer) and/or pre-cancerous conditions.

### BACKGROUND

Blood cancers such as leukemia (e.g., acute myelogenous leukemia or AML), Non-Hodgkin lymphoma (NHL), and multiple myeloma (MM) affect an estimated 1.2 million people in the US (Cancer Facts and Figures 2020) American Cancer Society). Of these, multiple myeloma (MM) is the second most common hematological malignancy that affects approximately 47,000 in United States annually (Shah D., Medscape (2021) April 15). Monoclonal gammopathy of undetermined significance (MGUS) and smoldering multiple myeloma (SMM) are monoclonal gammopathies which are of particular importance because MGUS or SMM patients are at risk for progression into MM or Waldenström macroglobulinemia (WM). See Perez-Persona et al. Blood (2007) 110, 2586-2592; Han, Jh., et al., Blood Cancer J. (2020) 10, 34. MM is associated with significant morbidity due to its end-organ destruction (Kazandjian D. Semin Oncol. (2016); 43(6):676-681). Although MM is treated using, e.g., chemotherapy alone or in combination with a bone marrow transplant, the prognosis for patients afflicted with multiple myeloma is generally poor. Efficacious therapies and/or prophylactic regimens for MM, WM and its associated pre-cancerous conditions are therefore urgently needed.

### SUMMARY

This disclosure relates to human Lymphocyte-activation gene 3 (LAG3) and human Galectin-3 (GAL3) inhibitory agents, and methods of their use either alone or in combination with (1) immunogenic X-Box Binding Protein 1 (XBP1), CD2 Subset 1 (CS1), and CD138 peptides, and/or (2) ex vivo activated immune cells specific for the XBP1/CS1/CD138 peptides, and/or (3) checkpoint inhibitors, for the treatment of cancer (e.g., blood cancer) and/or pre-cancerous conditions.

In a first aspect, this disclosure features a method of treating a human subject with a blood cancer or a pre-cancerous blood condition, the method comprising administering to the human subject a therapeutically effective amount of one or more inhibitory agents that inhibit interaction between human Lymphocyte-activation gene 3 (LAG3) and human Galectin-3 (GAL3).

In a second aspect, this disclosure features a method of treating a human subject with a pre-cancerous blood condition, a blood cancer, wherein precancerous cells or cancer cells of these conditions express one or more of X-Box Binding Protein 1 (XBP1), CD2 Subset 1 (CS1), and CD138, the method comprising administering to the human subject a combination of (a) a therapeutically effective amount of one or more inhibitory agents that inhibit the interaction between human LAG3 and human GAL3; with (b) at least one of (i) a multipeptide vaccine comprising a mixture of immunogenic peptides from one or more of XBP1, CS1, and CD138 that can induce antigen-specific T lymphocytes with anti-cancer activity; (ii) ex vivo activated XBP1, CS1, and/or CD138-specific T lymphocytes or peripheral blood mononuclear cells (PBMCs) with anti-cancer activity; and (iii) nanoparticles comprising a mixture of immunogenic peptides from one or more (1, 2, 3) of XBP1, CS1, and CD138 that can induce antigen-specific T lymphocytes with anti-cancer activity.

In a third aspect, this disclosure features a method for inhibiting progression from smoldering multiple myeloma (SMM) or monoclonal gammopathy of undermined significance (MGUS) to multiple myeloma (MM) in a human subject in need thereof, the method comprising administering to the human subject a combination of (a) a therapeutically effective amount of one or more inhibitory agents that inhibit the interaction between human LAG3 and human GAL3; with (b) at least one of (i) a multipeptide vaccine comprising a mixture of immunogenic peptides from one or more of XBP1, CS1, and CD138 that can induce antigen-specific T lymphocytes with anti-cancer activity;(ii) ex vivo activated XBP1, CS1, and/or CD138-specific T lymphocytes or peripheral blood mononuclear cells (PBMCs) with anti-cancer activity; and (c) nanoparticles comprising a mixture of immunogenic peptides from one or more (1, 2, 3) of XBP1, CS1, and CD138 that can induce antigen-specific T lymphocytes with anti-cancer activity.

In some cases of the second or third aspects above, peptide vaccination further comprises administering an adjuvant. In some cases, the adjuvant is incomplete Freund's adjuvant (IFA), Polyinosinic-polycytidylic acid, or poly-L-lysine (poly-ICLC)).

In certain instances of the second or third aspects above, the components of (a) and (b) of the combination are administered simultaneously, sequentially or, alternately. In some instances of the second or third aspects above, the components of (a) and (b) of the combination are administered multiple times during treatment.

In some instances of the second or third aspects above, the components of (a) and (b) of the combination are administered intravenously, intra-arterially, subcutaneously, intramuscularly, intraperitoneally, transdermally, orally, sublingually, intranasally, or transmucosally to the subject.

In some instances, the inhibitory agent is (a) an anti-LAG3 antibody that specifically binds to human LAG3; (b) an anti-GAL3 antibody that specifically binds to human GAL3; (c) a polypeptide comprising an extracellular domain of LAG3 that binds GAL3; or (d) a GAL3 inhibitor. In certain cases, the inhibitory agent triggers T lymphocyte proliferation in the human subject and/or Cluster of Differentiation 107a (CD107a) degranulation and/or Th1-type cytokine production. In some cases, the anti-LAG3 antibody and the anti-GAL3 antibody is a chimeric antibody, a humanized antibody, a bispecific antibody, or an antigen-binding fragment. In some cases, the anti-LAG3 antibody and the anti-GAL3 antibody is an antigen-binding fragment that is an Fab, F(ab)₂, a scFv, a sc(Fv)₂, or a diabody. In some instances, the Th1-type cytokine is one or more of interferon-γ (IFNγ), interleukin 2 (IL-2), IL-12, IL-18, and IL-27. For example, the Th1-type cytokine is IFNγ.

In some cases, the anti-LAG3 antibody is (i) an anti-LAG3 antagonistic antibody (optionally, Relatlimab, Encelimab, Favezelimab, Fianlimab, Ieramilimab, or Miptenalimab); (ii) an anti-LAG3 depleting antibody (optionally IMP731, or GSK2831781); or (iii) a soluble LAG3 immunoglobulin fusion protein (optionally Eftilagimod alpha); (iv) a bispecific antibody that binds LAG3 and Programmed cell death protein 1 (PD1), or a bispecific antibody that binds LAG3 and tumor necrosis factor receptor superfamily, member 4 (OX40), or a bispecific antibody that binds LAG3 and Glucocorticoid-Induced Tumor Necrosis Factor Receptor-Related (GITR). In some cases, the GAL3 inhibitor is (i) a small molecule inhibitor of LAG3 or GAL3; or (ii) GB0139 (TD139), belapectin, or modified citrus pectin.

In some instances, the anti-LAG3 antibody and/or the anti-GAL3 antibody has one or more of the following functions: (a) blocks interaction between LAG3 and GAL3; (b) the anti-LAG3 antibody competes with GAL3 for LAG3 binding and/or the anti-GAL3 antibody competes with LAG3 for GAL3 binding; (c) blocks LAG3 and/or GAL3 activation; or (d) blocks LAG3 and/or GAL3 signaling.

In some instances, the human subject is concurrently treated with one or more additional treatments, wherein the additional treatment is one or more forms of ionizing radiation and/or one or more agents selected from the group consisting of a therapeutic antibody, an immunomodulatory drug, a histone deacetylase (HDAC) inhibitor, an antineoplastic agent, a proteasome inhibitor, an antibody-drug conjugate, a nuclear export inhibitor; a corticosteroid. In some cases, the therapeutic antibody is selected from one or more of an anti-PD1 antibody (optionally Pembrolizumab or Nivolumab), an anti-PD-L1 antibody (optionally Durvalumab), an anti-CD38 antibody (optionally Daratumumab or Isatuximab), an anti-SLAMF7 antibody (optionally Elotuzumab), an anti-CTLA4 antibody (optionally Ipilimumab or Tremelimumab), an anti-TIM3 antibody (optionally Cobolimab), an anti-VISTA antibody (optionally SG7 or W0180), an anti-OX-40 antibody (optionally PF-04518600, or IBI101), and an anti-GITR antibody (optionally BMS-986156); the immunomodulatory drug is selected from one or more of lenalidomide, pomalidomide, and thalidomide; the HDAC inhibitor is citarinostat and/or panobinostat; the antineoplastic agent is selected from one or more of cyclophosphamide, etoposide, oxorubicin, liposomal doxorubicin, melphalan, melphalan flufenamide, and bendamustine; the proteasome inhibitor is selected from one or more of bortezomib, carfilzomib, and ixazomib; the antibody-drug conjugate is belantamab mafodotin-blmf; the nuclear export inhibitor is selinexor; and the corticosteroid is dexamethasone and/or prednisone.

In some instances, any of the above methods increase at least one of the following parameters in the human subject relative to a control population treated with the one or more additional treatments alone: (a) objective response rate (ORR); (b) time to next treatment (TTNT); (c) overall survival (OS); (d) progression free survival (PFS); and (e) the chance of achieving a negative minimal residual disease (MRD).

In some cases, the blood cancer is multiple myeloma (MM), leukemia, Non-Hodgkin lymphoma (NHL), or Waldenstrom's macroglobulinemia; and the pre-cancerous blood condition is smoldering multiple myeloma (SMM) or monoclonal gammopathy of undermined significance (MGUS). In some cases, the leukemia is acute myeloid leukemia (AML). The MM is active MM, newly diagnosed MM, relapsed MM, or relapsed/refractory MM.

In some instances of the second or third aspects above, the mixture of immunogenic peptides is selected from (a) one, two, three, or four HLA-A2-restricted peptides recited below: (i) a peptide of 50 amino acids or less in length comprising the amino acid sequence of non-spliced XBP1 set forth in SEQ ID NO: 24 (YISPWILAV) with 0, 1, 2, 3, or 4 substitutions, wherein the non-spliced XBP1 peptide binds to HLA-A2; (ii) a peptide of 50 amino acids or less in length and comprising the amino acid sequence of spliced XBP1 set forth in SEQ ID NO: 25 (YLFPQLISV) with 0, 1, 2, 3, or 4 substitutions, wherein the spliced XBP1 peptide binds to HLA-A2; (iii) a peptide of 50 amino acids or less in length and comprising the amino acid sequence of CD138 peptide set forth in SEQ ID NO: 26 (GLVGLIFAV) with 0, 1, 2, 3, or 4 substitutions, wherein the CD138 peptide binds to HLA-A2; and (iv) a peptide of 50 amino acids or less in length comprising the amino acid sequence of CS-1 set forth in SEQ ID NO: 27 (SLFVLGLFL) with 0, 1, 2, 3, or 4 substitutions, wherein the CS-1 peptide binds to HLA-A2; or (b) one, two, three, or four HLA-A24-restricted peptides recited below (i) a peptide of 50 amino acids or less in length comprising the amino acid sequence of non-spliced XBP1 set forth in SEQ ID NO: 28 (ISPWILAVL) with 0, 1, 2, 3, or 4 substitutions, wherein the non-spliced XBP1 peptide binds to HLA-A24; (ii) a peptide of 50 amino acids or less in length and comprising the amino acid sequence of spliced XBP1 set forth in SEQ ID NO: 29 (VYPEGPSSL) with 0, 1, 2, 3, or 4 substitutions, wherein the spliced XBP1 peptide binds to HLA-A24; (iii) a peptide of 50 amino acids or less in length and comprising the amino acid sequence of CD138 set forth in SEQ ID NO: 30 (IFAVCLVGF) with 0, 1, 2, 3, or 4 substitutions, wherein the CD138 peptide binds to HLA-A24; and (iv) a peptide of 50 amino acids or less in length comprising the amino acid sequence of CS-1 set forth in SEQ ID NO: 32 (LFVLGLFLW) with 0, 1, 2, 3, or 4 substitutions, wherein the CS-1 peptide binds to HLA-A24.

In some instances of the second or third aspects above, the mixture of immunogenic peptides is selected from (a) one, two, three, or four of HLA-A2-restricted peptides recited below: (i) a peptide comprising the amino acid sequence of non-spliced XBP1 set forth in SEQ ID NO: 24 (YISPWILAV); (ii) a peptide comprising the amino acid sequence of spliced XBP1 set forth in SEQ ID NO: 25 (YLFPQLISV); (iii) a peptide comprising the amino acid sequence of CD138 set forth in SEQ ID NO: 26 (GLVGLIFAV); and (iv) a peptide of comprising the amino acid sequence of CS-1 set forth in SEQ ID NO: 27 (SLFVLGLFL); or (b) one, two, three, or four of HLA-A24-restricted peptides recited below: (i) a peptide comprising the amino acid sequence of non-spliced XBP1 set forth in SEQ ID NO: 28 (ISPWILAVL); (ii) a peptide comprising the amino acid sequence of spliced XBP1 set forth in SEQ ID NO: 29 (VYPEGPSSL); (iii) a peptide comprising the amino acid sequence of CD138 set forth in SEQ ID NO: 30 (IFAVCLVGF); and (iv) a peptide comprising the amino acid sequence of CS-1 set forth in SEQ ID NO: 31 (LFVLGLFLW).

In some instances of the second or third aspects above, the mixture of immunogenic peptides is selected from (a) one, two, three, or four of HLA-A2-restricted peptides recited below: (i) a peptide consisting of the amino acid sequence of non-spliced XBP1 set forth in SEQ ID NO: 24 (YISPWILAV); (ii) a peptide consisting of the amino acid sequence of spliced XBP1 set forth in SEQ ID NO: 25 (YLFPQLISV); (iii) a peptide consisting of the amino acid sequence of CD138 set forth in SEQ ID NO: 26 (GLVGLIFAV); and (iv) a peptide of consisting of the amino acid sequence of CS-1 set forth in SEQ ID NO: 27 (SLFVLGLFL); or (b) one, two, three, or four of HLA-A24-restricted peptides recited below: (i) a peptide consisting of the amino acid sequence of non-spliced XBP1 set forth in SEQ ID NO: 28 (ISPWILAVL); (ii) a peptide consisting of the amino acid sequence of spliced XBP1 set forth in SEQ ID NO: 29 (VYPEGPSSL); (iii) a peptide consisting of the amino acid sequence of CD138 set forth in SEQ ID NO: 30 (IFAVCLVGF); and (iv) a peptide consisting of the amino acid sequence of CS-1 set forth in SEQ ID NO: 31 (LFVLGLFLW).

In some instances, the ex vivo activated T lymphocytes are generated by the following steps: (a) providing or isolating T lymphocytes and antigen presenting cells from the human subject or an HLA-matched donor; (b) contacting the antigen presenting cells with a multipeptide vaccine comprising three or more of a non-spliced XBP1 peptide, a spliced XBP1 peptide, a CD138 peptide, and a CS-1 peptide; and (c) contacting the T lymphocytes with the antigen presenting cells from step (b) to generate ex vivo activated T lymphocytes.

In some instances, the ex vivo activated PBMCs are generated by the following steps: (a) providing or isolating PBMCs from the human subject or an HLA-matched donor; (b) contacting the PBMCs with a mixture of immunogenic peptides comprising three or more of a non-spliced XBP1 peptide, a spliced XBP1 peptide, a CD138 peptide, and a CS-1 peptide; and (c) generating ex vivo activated PBMCs.

In some instances, the human subject with the pre-cancerous blood condition does not develop MM.

In another aspect, the disclosure provides a method for increasing T lymphocyte responses in a tumor microenvironment while reducing immunosuppression in a human subject in need thereof, the method comprising administering a combination of (a) a therapeutically effective amount of one or more inhibitory agents that inhibit the interaction between human LAG3 and human GAL3; with (b) a therapeutically effective amount of one or more of an anti-PD1 antibody, an anti-OX40 antibody, and an anti-GITR antibody. In some instances, the inhibitory agent is an anti-LAG3 antibody or an anti-GAL3 antibody. For example, the anti-LAG3 antibody is Relatlimab and the anti-PD1 antibody is Pembrolizumab or Nivolumab.

In yet another aspect, the disclosure provides a pharmaceutical composition comprising (a) any one or a mixture of two or more immunogenic peptides, wherein the immunogenic peptides comprise the amino acid sequence of a non-spliced XBP1 peptide, a spliced XBP1 peptide, a CD 138 peptide, and a CS1 peptide; (b) a LAG3 inhibitory agent and/or a GAL3 inhibitory agent; and (c) a pharmaceutically acceptable carrier.

In another aspect, the disclosure provides a combination comprising (a) a multipeptide vaccine comprising at least three immunogenic peptides, wherein the immunogenic peptides comprise the amino acid sequence of a non-spliced XBP1 peptide, a spliced XBP1 peptide, a CD 138 peptide, and a CS-1 peptide; and (b) a composition comprising a LAG3 inhibitory agent and/or a GAL3 inhibitory agent. In yet another aspect, the disclosure provides a combination comprising (a) means for targeting HLA-A2+ or HLA-A24+ pre-cancerous or cancerous cells that express one or more of XBP1, CS1, and CD138, and (b) a LAG3 inhibitory agent and/or a GAL3 inhibitory agent. In another aspect, the disclosure provides a combination comprising (a) anti-myeloma-specific T lymphocytes targeting XBP1, CD138 and CS1-expressing cells from SMM, MGUS, or MM patients and (b) means for specifically binding human LAG3 and/or means for specifically binding human GAL3. In yet another aspect, the disclosure provides a combination comprising (a) anti-myeloma-specific PBMCs targeting XBP1, CD138 and CS1-expressing cells from SMM, MGUS, or MM patients and (b) means for specifically binding human LAG3 and/or means for specifically binding human GAL3. In another aspect, the disclosure provides a combination comprising (a) nanoparticles comprising a mixture of immunogenic peptides from one or more (1, 2, 3) of XBP1, CS1, and CD138 and (b) means for specifically binding human LAG3 and/or means for specifically binding human GAL3. In some instances of any of the above combinations, (a) and (b) are formulated for administration to a human subject in need thereof, simultaneously, sequentially or, alternately.

In yet another aspect, the disclosure provides a kit comprising (a) a first composition comprising at least three of a non-spliced XBP1 peptide, a spliced XBP1 peptide, a CD 138 peptide and a CS-1 peptide, (b) a second composition comprising an anti-LAG3 antibody and/or an anti-GAL3 antibody, and optionally, (c) instructions for administering the first and second compositions to a subject. In some instances, the kit further comprises one or more additional therapeutic agents.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIGS. 1A-1C show proliferation of specific T cell subsets as measured by a Carboxy Fluorescein Succinimidyl Ester (CFSE) assay and acquired by flow cytometry. Bone marrow mononuclear cells (BMMC) from Multiple Myeloma (MM) patients (newly diagnosed, relapsed, relapsed/refractory) were treated with low dose (20 units) IL-2 and evaluated for proliferation. FIG. 1A shows proliferation of total CD3+ T cells, and CD3+ T cells expressing PD1, LAG3, OX40 or GITR in cultures of BMMC from MM patients (N=10). FIG. 1B shows proliferation of CD3+ T cells, CD4+ Th cells and CD8+ Tc cells with anti-PD1, anti-LAG3, anti-OX40 or anti-GITR treatment. FIG. 1C shows proliferation of CD3+ T cells in newly diagnosed (N=6) or relapsed (N=3) MM patients PBMC with anti-LAG3 or anti-OX40 treatment, with or without MM lysates stimulation. (*p<0.05)

FIGS. 2A-2B show characterization of regulatory T cells in BMMC or PBMC from patients with smoldering multiple myeloma (SMM), monoclonal gammopathy of undermined significance (MGUS), or MM and healthy individuals. Freshly isolated BMMC or peripheral blood mononuclear cells (PBMCs) from patients with MGUS (BM: N=5, PB: N=5), SMM (BM: N=5, PB: N=5), newly diagnosed MM (BM: N=5, PB: N=5), relapsed MM (BM: N=5, PB: N=5), relapsed/refractory MM (BM: N=5, PB: N=5), and healthy donors (BM: N=4, PB: N=5) were evaluated for the frequency of regulatory T cells and their expression of immune checkpoints using flow cytometry. FIG. 2A shows CD4+ Treg (CD25+FOXP3+/CD3+CD4+) cells in BMMC and PBMCs in active MM patients, MGUS/SMM patients or healthy individuals. FIG. 2B shows PD1, LAG3 or GITR expression in CD4+ Treg cells from active MM patients, MGUS/SMM patients or healthy individuals. (*p<0.05)

FIGS. 3A-3C show characterization of Myeloid-derived suppressor cells (MDSCs) and CD138+ MM cells in BMMC or PBMC from patients with MGUS, SMM or MM and healthy donors. Freshly isolated BMMC or PBMC from patients with MGUS (BM: N=5, PB: N=5), SMM (BM: N=5, PB: N=5), newly diagnosed MM (BM: N=5, PB: N=5), relapsed MM (BM: N=5, PB: N=5), relapsed/refractory MM (BM: N=5, PB: N=5) and healthy donors (BM: N=4, PB: N=5) were evaluated for the frequency of MDSC or CD138+MM cells and their expression of immune checkpoints using flow cytometry. FIG. 3A shows G-type MDSC (CD11b+CD33+ HLA-DRlow/−CD14−CD15+), and M-type MDSC (CD11b+CD33+ HLA−DRlow/−CD14+CD15−) cell populations from active MM patients, MGUS/SMM patients or healthy individuals. FIG. 3B shows PD-L1, PD-L2 or LAG3 cell populations in G-type MDSC from BMMC and PBMC in active MM patients, MGUS/SMM patients or healthy individuals. FIG. 3C shows PD-L1 and PD-L2 expression in CD138+ tumor cells in MM patients' BMMCs and healthy individual BMMCs. (*p<0.05)

FIGS. 4A-4B show distribution, location and expression level of key immune checkpoints, activation and costimulatory molecules in MM patients' BMMCs. Cell surface and intracellular expression of immune checkpoints were evaluated and compared in BMMCs from MM patients (newly diagnosed, relapsed, relapsed/refractory; N=9) using flow cytometry. FIG. 4A shows surface and intracellular expression of CTLA4, PD1, LAG3, and TIM3 in CD3+ T cells in BMMCs from MM patients. FIG. 4B shows surface and intracellular expression of CTLA4, PD1, GAL-9 and ICOS-L in CD138+ tumor cells in BMMCs from MM patients. (*p<0.05)

FIGS. 5A-5C show the impact of checkpoint inhibitor or immune agonist treatment on MM patients' BMMCs. BMMCs from MM patients were treated with a checkpoint inhibitor or immune agonist in the presence of low level of IL-2 (20 units/ml) and examined for checkpoint expression and immune function using flow cytometry. FIG. 5A shows proliferation of PD1+ or LAG3+ T cells when BMMCs from MM patients (N=10) were treated with anti-PD1, anti-LAG3, anti-OX40 or anti-GITR antibodies. FIG. 5B shows CD4+ Treg proliferation when BMMC of MM patients (N=5) were treated with anti-PD1, anti-LAG3, anti-OX40 or anti-GITR antibodies. FIG. 5C shows Treg proliferation when BMMC from MM patients (N=3) were treated with anti-PD1, anti-LAG3, anti-OX40 or anti-GITR antibodies or combinations thereof as indicated. (*p<0.05)

FIGS. 6A-6C show the impact of checkpoint inhibitor treatment on anti-MM activities of XBP1/CD138/CS1-specific CTL. HLA-A2-specific XBP1/CD138/CS1− specific CTL (N=5) were generated by four cycles of weekly stimulation of CD3+ T cells with immunogenic XBP1/CD138/CS1 peptides and then examined for their phenotypic profiles and functional activities against MM using flow cytometry. FIG. 6A shows T cell activation (CD69) and checkpoint (CTLA4, PD1, LAG3, VISTA, TIM3) expression on MM-specific CTL at the indicated times post the fourth cycle of multi-peptide stimulation. FIG. 6B shows induced proliferation of total CD8+ CTL as well as central memory, effector memory, CD28+ and CD38+ CTL subsets; and FIG. 6C shows CD107a+ degranulation and IFN-γ production; in XBP1/CD138/CS1-specific CTLs which were untreated, or treated with immune checkpoint inhibitors anti-LAG3 antibody or anti-PD1 antibody, in response to HLA-A2-matched U266 MM cells.

FIGS. 7A-7C show the phenotype and functional characterization of the LAG3 ligands, GAL-3 and HLA-DP/DQ/DR on CD138+ cells in BMMCs or PBMCs from MM patients, evaluated by flow cytometry. FIG. 7A shows GAL-3 and HLA-DP/DQ/DR intracellular and cell surface expression in CD138+ tumor cells from MM patients (N=4). FIG. 7B shows proliferation of CD8+Tc cells and CD4+Th cells after treatment of MM patients' BMMCs with anti-GAL-3 and anti-HLA-DP/DQ/DR antibodies. FIG. 7C shows proliferation of CD8+Tc cells and CD4+Th cells in BMMCs from MM patients (N=5) with anti-GAL-3 and anti-HLA-DP/DQ/DR antibodies (*p<0.05).

FIGS. 8A-8C shows the impact of inhibition of LAG3 ligands on proliferation and anti-tumor activities analyzed by flow cytometry using XBP1/CD138/CS1-specific CD8+ CTL against MM cells. FIG. 8A shows intracellular and cell surface expression of GAL-3 and HLA-DP/DQ/DR in CD138+ tumor cells of MM cell lines (N=3). FIG. 8B shows proliferation of MM-specific CD8+ CTLs in response to HLA-A2+U266 MM cells, treated with anti-GAL-3 and anti-HLA-DP/DQ/DR antibodies. The XBP1/CD138/CS1-CTL: MM cells ratios used were 1:1, 1:0.5, and 1:0.25). FIG. 8C shows proliferation of LAG+ cells triggered in XBP1/CD138/CS1-specific CTLs by stimulation with U266 treated with anti-GAL-3 and anti-HLA-DP/DQ/DR antibodies. The XBP1/CD138/CS1-CTL: MM cells ratios used were 1:1, 1:0.5, 1:0.25, and 1:0. (*p<0.05)

FIG. 9 shows proliferation of CD3+ T cells, CD4+ T cells and CD8+ T cells in BMMC cells stimulated with irradiated tumor cells or tumor lysates and treated with anti-LAG3, anti-PD1, anti-OX40, and anti-GITR antibodies. (*p<0.05)

### DETAILED DESCRIPTION

This disclosure is based, in part, on the findings that (1) proliferating T cells in bone marrow mononuclear cells or peripheral blood mononuclear cells from patients with multiple myeloma upregulate a key checkpoint, Lymphocyte-activation gene 3 (LAG3); (2) Galectin-3 (GAL3), the ligand for LAG3, is robustly expressed on CD138+MM cells, (3) checkpoint inhibitor anti-LAG3 treatment enhances T cell responses in the tumor microenvironment; (4) blocking of LAG3 or its ligand GAL3 effectively triggers T cell proliferation in MM patients' BMMC/PBMC and anti-MM activities (CD107a degranulation, Th1-type cytokine production) of X-Box Binding Protein 1 (XBP1), CD2 Subset 1 (CS1), and/or CD138-specific memory CTL; (5) combination treatments with antibodies to Programmed cell death protein 1 (PD1), LAG3, tumor necrosis factor receptor superfamily, member 4 (OX40) and Glucocorticoid-Induced Tumor Necrosis Factor Receptor-Related (GITR) enhanced more robust immune responses, with less suppressors in MM patients' BMMCs and (6) blocking LAG3 or its ligand GAL3 augments anti-tumor activity of XBP1/CD138/CS1-specific memory CD8+ CTLs in multiple myeloma.

Thus, this disclosure features inhibitory agents to human LAG3 and human GAL3, which can be used to induce anti-tumor activity (e.g., trigger T cell proliferation), as well as immunogenic XBP1-, CD138-, and/or CS-1-derived peptides, which can be used to induce an immune response (e.g., stimulate a cytotoxic T cell response) in a subject (e.g., human). The LAG3/GAL3 inhibitory agents alone or in combination with (a) antibodies to PD1, LAG3, OX40 and GITR and/or (b) the XBP1/CD138/CS1 peptides (e.g., a multipeptide vaccine) and/or (c) ex vivo immune cells activated with XBP1/CD138/CS1 peptides of this disclosure, and/or (d) nanoparticles comprising a mixture of immunogenic peptides from one or more (1, 2, 3) of XBP1, CS1, and CD138 that can induce antigen-specific T lymphocytes with anti-cancer activity, can be used in a variety of applications such as methods for treating a cancer such as a blood cancer (e.g., multiple myeloma (MM), Waldenstrom's macroglobulinemia (WM), leukemia (e.g., acute myelogenous leukemia (AML), or Non-Hodgkin lymphoma) or a pre-cancerous state such as a pre-cancerous blood condition (e.g., smoldering multiple myeloma (SMM) or monoclonal gammopathy of undermined significance (MGUS)), methods for inhibiting progression from SMM or MGUS to MM, or methods for increasing T lymphocyte responses in a tumor microenvironment while reducing immunosuppression. The disclosure also features pharmaceutical compositions of the LAG3/GAL3 inhibitory agents and the immunogenic XBP1/CD138 and/or CS1 peptides and combinations of the LAG3/GAL3 inhibitory agents with multipeptide vaccines; and/or ex vivo activated immune cells specific for XBP1/CD138 and/or CS1 peptides; and/or nanoparticles comprising a mixture of immunogenic peptides from one or more (1, 2, 3) of XBP1, CS1, and CD138.

A detailed description of the LAG3/GAL3 inhibitory agents and the immunogenic XBP1/CD138 and/or CS1 peptides, as well as methods of using these agents and peptides, as well as compositions and combinations thereof, are set forth below.

### Human LAG3 and LAG3 Inhibitory Agents

Human Lymphocyte Activated Gene-3 (LAG3; CD223) is a cell surface molecule expressed on activated T cells, NK cells, B cells, and dendritic cells, and plays an important role in the function of these lymphocyte subsets. See, e.g., Maruhashi T et al., Journal for ImmunoTherapy of Cancer 2020; 8:e001014. Doi: 10.1136/jitc-2020-001014; and U.S. Ser. No. 10/188,730, incorporated by reference herein). LAG3 is an immune checkpoint that regulates T cell function in a tumor environment. It is a co-inhibitory receptor which suppresses T cells activation and cytokine secretion. LAG-3 binds to major histocompatibility complex-II (MHC-II) on antigen presenting cells (APCs), and also binds to Galectin-3 (GAL3), widely expressed in different cell types. See, e.g., Long L. et al., Genes & Cancer, (2018); 9(5-6): 176-189.

The term “LAG3”, with respect to the polypeptide to which inhibitory agents of the disclosure bind, refers to human LAG3, as well as fragments thereof such as the mature fragment thereof lacking the signal peptide that are still capable of binding its ligands (e.g., GAL3).

Human LAG3 has two isoforms, the amino acid sequences of which are as follows:

human LAG3 Isoform-1
(Uniprot accession no. P18627-1):
SEQ ID NO: 1
MWEAQFLGLLFLQPLWVAPVKPLQPGAEVPVVWAQEGAPAQLPCS
PTIPLQDLSLLRRAGVTWQHQPDSGPPAAAPGHPLAPGPHPAAPS
SWGPRPRRYTVLSVGPGGLRSGRLPLQPRVQLDERGRQRGDFSLW
LRPARRADAGEYRAAVHLRDRALSCRLRLRLGQASMTASPPGSLR
ASDWVILNCSFSRPDRPASVHWFRNRGQGRVPVRESPHHHLAESF
LFLPQVSPMDSGPWGCILTYRDGFNVSIMYNLTVLGLEPPTPLTV
YAGAGSRVGLPCRLPAGVGTRSFLTAKWTPPGGGPDLLVTGDNGD
FTLRLEDVSQAQAGTYTCHIHLQEQQLNATVTLAIITVTPKSFGS
PGSLGKLLCEVTPVSGQERFVWSSLDTPSQRSFSGPWLEAQEAQL
LSQPWQCQLYQGERLLGAAVYFTELSSPGAQRSGRAPGALPAGHL
LLFLILGVLSLLLLVTGAFGFHLWRRQWRPRRFSALEQGIHPPQA
QSKIEELEQEPEPEPEPEPEPEPEPEPEQL
human LAG3 Isoform-2
(Uniprot accession no. P18627-2):
SEQ ID NO: 2
MWEAQFLGLLFLQPLWVAPVKPLQPGAEVPVVWAQEGAPAQLPCS
PTIPLQDLSLLRRAGVTWQHQPDSGPPAAAPGHPLAPGPHPAAPS
SWGPRPRRYTVLSVGPGGLRSGRLPLQPRVQLDERGRQRGDFSLW
LRPARRADAGEYRAAVHLRDRALSCRLRLRLGQASMTASPPGSLR
ASDWVILNCSFSRPDRPASVHWFRNRGQGRVPVRESPHHHLAESF
LFLPQVSPMDSGPWGCILTYRDGFNVSIMYNLTVLGLEPPTPLTV
YAGAGSRVGLPCRLPAGVGTRSFLTAKWTPPGGGPDLLVTGDNGD
FTLRLEDVSQAQAGTYTCHIHLQEQQLNATVTLAIITGQPQVGKE

An “inhibitory agent” is an agent that blocks the activity of LAG3, and/or inhibits the interaction between LAG3 and its ligand, GAL3. In some embodiments, a LAG3 inhibitory agent is an “anti-LAG3 antibody” (e.g., an antagonistic antibody) or an antigen binding fragment thereof. An antagonist anti-LAG3 antibody or antigen-binding fragment thereof antagonizes an activity of LAG3 (e.g., human LAG3) such as by inhibiting LAG3 binding to GAL3; competing with GAL3 for LAG3 binding; or when a cell or subject is contacted with the anti-LAG3 antibody or fragment thereof, biological activity associated with LAG3 antagonism occurs, such as proliferation of antigen-specific T-cells, CD107a degranulation, and/or stimulation of Th1-type cytokine production (e.g., interferon-γ (IFNγ) IL-2, IL-12, IL-18, IL-27. In some embodiments, a LAG3 inhibitory agent is a polypeptide comprising soluble LAG3 (that can bind GAL3) or a polypeptide comprising a soluble LAG3-immunoglobulin fusion (e.g., a human Fc fusion such as a human IgG1 Fc fusion). In some embodiments, the LAG3 inhibitory agent is an extracellular domain of LAG3 that binds GAL3. GAL-3 is a lectin with carbohydrate-recognition domains (which may bind to glycosylated sites of LAG3) and oligomerization domains that facilitate LAG3 cross-linking (Graydon, C G et al. Frontiers in Immunology (2020) 11:615317). In some embodiments, the LAG3 inhibitory agent blocks the binding of GAL3 to carbohydrates on LAG3 (e.g. glycosylated sites) and/or block cross-linking of LAG3. In some embodiments, the LAG3 inhibitory agent is a small molecule inhibitor of LAG3 (see, e.g., Rudd et al., Cell Reports, (2020) Volume 30, Issue 7, Pages 2075-2082.e4).

An “anti-LAG3 antibody” that specifically binds to human LAG3 may be a chimeric antibody, a humanized antibody, a bispecific antibody, or an antigen-binding (meaning LAG3-binding) fragment. The antigen-binding fragment may be an Fab, F(ab)₂, a scFv, a sc(Fv)₂, or a diabody. Several anti-LAG3 antibodies are known in the art and may be used in the methods of this disclosure. These include, but are not limited to, anti-LAG3 antagonistic antibodies such as Relatlimab, Encelimab, Favezelimab, Fianlimab, Ieramilimab, or Miptenalimab); anti-LAG3 depleting antibodies, such as IMP731 or GSK2831781); a bispecific antibody that binds LAG3 and Programmed cell death protein 1 (PD1), a bispecific antibody that binds LAG3 and tumor necrosis factor receptor superfamily, member 4 (OX40), or a bispecific antibody that binds LAG3 and Glucocorticoid-Induced Tumor Necrosis Factor Receptor-Related (GITR). Such antibodies are reviewed in Shan C et al; Oncology Letters (2020) 20:207; and Long L. et al., Genes & Cancer, (2018); 9(5-6): 176-189.

In some instances, a LAG3 inhibitory agent is a soluble LAG3 immunoglobulin fusion protein, such as Eftilagimod alpha, which is a 160 kDa protein consisting of the four extracellular domains of LAG-3 fused to the Fc region of an IgG1(LAG-3Ig).

In some embodiments, a LAG3 inhibitory agent is an Fc-bearing bispecific tetravalent DART® protein (Tebotelimab) that blocks PD-1 and LAG-3 checkpoint molecules. In some embodiments, a LAG3 inhibitory agent is a bispecific, tetravalent antibody (mAb) against LAG-3 and PD-L1. See, e.g., Kraman M. et al, Clin Cancer Res (2020) 26:(13) 3333-3344.

In some embodiments, the anti-LAG3 antibody of the disclosed compositions and methods has the light chain and heavy chain CDRs, the variable heavy and light chains variable regions, and the heavy and light chains disclosed in U.S. Pat. Nos. U.S. Pat. No. 9,505,839, U.S. Ser. No. 10/188,730, U.S. Ser. No. 10/358,495, and U.S. Patent Application Publication No. US20200277372, the disclosures of which are incorporated by reference herein in their entirety. For example, the anti-LAG3 antibody can have the following six CDR sequences, VH, VL, and H/L chains as shown below in Table 1, which correspond to SEQ ID NOs: 12, 14, 15-20, of U.S. Pat. No. 9,505,839:

TABLE 1
anti-LAG3 antibody (Reatlimab) CDR,
VH, VL, H, L sequences

| SEQ | | |
|---|---|---|
| ID | Type of | |
| NO: | sequence | Sequence |
| 3 | Light chain | DYYWN |
| | CDR1 | |
| 4 | Light chain | EINHRGSTNSNPSLKS |
| | CDR2 | |
| 5 | Light chain | GYSDYEYNWFDP |
| | CDR3 | |
| 6 | Heavy chain | RASQSISSYLA |
| | CDR1 | |
| 7 | Heavy chain | DASNRAT |
| | CDR2 | |
| 8 | Heavy chain | QQRSNWPLT |
| | CDR3 | |
| 9 | Heavy chain | QVQLQQWGAGLLKPSETLSLTCAVYGGSFSDYYW |
| | variable | NWIRQPPGKGLEWIGEINHRGSTNSNPSLKSRVT |
| | region | LSLDTSKNQFSLKLRSVTAADTAVYYCAFGYSDY |
| | (VH) | EYNWFDPWGQGTLVTVSS |
| 10 | Light chain | EIVLTQSPATLSLSPGERATLSCRASQSISSYLA |
| | variable | WYQQKPGQAPRLLIYDASNRATGIPARFSGSGSG |
| | region | FTDTLTISSLEPEDFAVYYCQQRSNWPLTFGQGT |
| | (VH) | NLEIK |
| 11 | Heavy chain | QVQLQQWGAGLLKPSETLSLTCAVYGGSFSDYY |
| | complete | WNWIRQPPGKGLEWIGEINHRGSTNSNPSLKSR |
| | sequence | VTLSLDTSKNQFSLKLRSVTAADTAVYYCAFGY |
| | | SDYEYNWFDPWGQGTLVTVSSASTKGPSVFPLA |
| | | PCSRSTSESTAALGCLVKDYFPEPVTVSWNSGA |
| | | LTSGVHTFPAVLQSSGLYSLSSVVTVPSSSLGT |
| | | KTYTCNVDHKPSNTKVDKRVESKYGPPCPPCPA |
| | | PEFLGGPSVFLFPPKPKDTLMISRTPEVTCVVV |
| | | DVSQEDPEVQFNWYVDGVEVHNAKTKPREEQFN |
| | | STYRVVSVLTVLHQDWLNGKEYKCKVSNKGLPS |
| | | SIEKTISKAKGQPREPQVYTLPPSQEEMTKNQV |
| | | SLTCLVKGFYPSDIAVEWESNGQPENNYKTTPP |
| | | VLDSDGSFFLYSRLTVDKSRWQEGNVFSCSVMH |
| | | EALHNHYTQKSLSLSLGK |
| 12 | Light chain | EIVLTQSPATLSLSPGERATLSCRASQSISSYL |
| | complete | AWYQQKPGQAPRLLIYDASNRATGIPARFSGSG |
| | sequence | SGTDFTLTISSLEPEDFAVYYCQQRSNWPLTFG |
| | | QGTNLEIKRTVAAPSVFIFPPSDEQLKSGTASV |
| | | VCLLNNFYPREAKVQWKVDNALQSGNSQESVTE |
| | | QDSKDSTYSLSSTLTLSKADYEKHKVYACEVTH |
| | | QGLSSPVTKSFNRGEC |

In some embodiments, any LAG3 antibody that inhibits LAG-3 binding to GAL3 can be used in the compositions and methods of the disclosure. In some instances, art recognized anti-LAG-3 antibodies can be used in the compositions and methods of the disclosure. For example, the anti-human LAG-3 antibody described in US2011/0150892 A1, and referred to as monoclonal antibody 25F7 can be used. Other art recognized anti-LAG-3 antibodies that can be used include IMP731 (H5L7BW) described in US 2011/007023, MK-4280 (28G-10) described in WO2016028672, aLAG3(0414) and aLAG3(0416) described in WO2018185046, anti-PD1/LAG3 0927 described in WO2018185043, REGN3767 described in Journal for ImmunoTherapy of Cancer, (2016) Vol. 4, Supp. Supplement 1 Abstract Number: P195, BAP050 described in WO2017/019894, IMP-701 (LAG-525), aLAG3(0414), aLAG3(0416), Sym022, TSR-033, TSR-075, XmAb22841, MGD013, BI754111, FS118, P 13B02-30, AVA-017 and GSK2831781. These and other anti-LAG-3 antibodies useful in the claimed invention can be found in, for example: WO2016/028672, WO2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018185046, WO2018185043, and WO2014/140180. The contents of each of these references are incorporated by reference herein in their entirety. In some embodiments, the anti-LAG3 antibody binds to specific epitopes within a LAG3 protein as described in any of the above references.

In some embodiments, the monotherapy or combination therapies herein comprise administering an antibody molecule related to Relatlimab (e.g., in place of Relatlimab in any of the methods herein). In some embodiments, the antibody molecule comprises a heavy chain sequence having at least 70%, 80%, 85%, 90%, 95%, 96%, 97%, 98%, or 99% identity to SEQ ID NO: 11. In some embodiments, the antibody molecule comprises a light chain sequence having at least 70%, 80%, 85%, 90%, 95%, 96%, 97%, 98%, or 99% identity to SEQ ID NO: 12. In embodiments, the antibody molecule comprises a VH region having at least 70%, 80%, 85%, 90%, 95%, 96%, 97%, 98%, or 99% identity to SEQ ID NO: 9, or having the VH region of SEQ ID NO: 9. In embodiments, the antibody molecule comprises a VL region having at least 70%, 80%, 85%, 90%, 95%, 96%, 97%, 98%, or 99% identity to the VL region of SEQ ID NO: 10, or having the VL region from SEQ ID NO: 10. In embodiments, the antibody molecule comprises the HC CDR1, HC CDR2, and HC CDR3 of SEQ ID NOs: 3-5 and a LC CDR1, LC CDR2, and LC CDR3 of SEQ ID NOs: 6-8. In some instances, the antibody molecule comprises the HC CDR1, HC CDR2, and HC CDR3 of SEQ ID NO:9 and the LC CDR1, LC CDR2, and LC CDR3 of SEQ ID NO: 10. In some cases, the CDRs are defined according to Kabat, Chothia, enhanced Chothia, contact, IMGT, AbM, or the Abysis definitions.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al Lazikani et al.,(1997) JMB 273,927-948 (“Chothia” numbering scheme), or a combination thereof.

### Human GAL3 and GAL3 Inhibitory Agents

Human Galectin3 (Gal3) is a lectin, or a carbohydrate-binding protein, with specificity towards beta-galactosides. In human cells, Gal3 is expressed and can be found in the nucleus, cytoplasm, cell surface, and in the extracellular space. It is expressed in multiple myeloma CD138+ cells and Galectin-3 is associated with tumor cell adhesion, proliferation, differentiation, angiogenesis, and metastasis (Storti, Paola et al. International J. Mol. Sci 18(12):2740 (2017); Chauhan, D. et al, Cancer Res Sept 2005 (65) (18) 8350-8358).

The term “GAL3”, with respect to the polypeptide to which inhibitory agents of the disclosure bind, refers to human GAL3. In some embodiments, the anti-GAL3 antibody binds to specific epitopes within a GAL3 protein as described in U.S. Patent Application Publ. No. US20200223921 or International Patent Appl Publ. No. WO2020160156, incorporated by reference in its entirety.

The amino acid sequences of human GAL3 is as follows:

human GAL3 Isoform-1
(Uniprot accession no. P17931-1):
SEQ ID NO: 13
MADNFSLHDALSGSGNPNPQGWPGAWGNQPAGAGGYPGASYPGAY
PGQAPPGAYPGQAPPGAYPGAPGAYPGAPAPGVYPGPPSGPGAYP
SSGQPSATGAYPATGPYGAPAGPLIVPYNLPLPGGVVPRMLITIL
GTVKPNANRIALDFQRGNDVAFHFNPRFNENNRRVIVCNTKLDNN
WGREERQSVFPFESGKPFKIQVLVEPDHFKVAVNDAHLLQYNHR
VKKLNEISKLGISGDIDLTSASYTMI

A “GAL3 inhibitory agent” is an agent that blocks the activity of GAL3, and/or inhibits the interaction between LAG3 and GAL3. In some embodiments, a GAL3 inhibitory agent is an “anti-GAL3 antibody” or an antigen binding fragment thereof. In some embodiments, the anti-GAL3 antibody of the disclosed compositions and methods has the light chain and heavy chain CDRs, the variable heavy and light chains variable regions, and the heavy and light chains disclosed in U.S. Patent Application Publ. No. US20200223921, and International Patent Publ. No. WO2020160156 the disclosures of which are incorporated by reference herein in their entirety. For example, the anti-GAL3 antibody has following six CDR sequences, VH, VL, and H/L chains as shown below in Table 2, which correspond to SEQ ID NOs: 9-11, 16-19, and 24-26 of US Patent Application Publ. No. US20200223921:

TABLE 2
anti-GAL3 CDR, VH, VL, H, L sequences

| SEQ | | |
|---|---|---|
| ID | Type of | |
| NO: | sequence | Sequence |
| 14 | Light chain | RSSKSLLYKDGKTYLN |
| | CDR1 | |
| 15 | Light chain | LMSTHAS |
| | CDR2 | |
| 16 | Light chain | QQLVDYPLT |
| | CDR3 | |
| 17 | Heavy chain | GYTFTNY |
| | CDR1 | |
| 18 | Heavy chain | NTNTGE |
| | CDR2 | |
| 19 | Heavy chain | YDNFFAY |
| | CDR3 | |
| 20 | Heavy chain | QVQLVQSGSELKKPGASVKVSCKASGYTFTN |
| | variable | YGMNWVRQAPGQGLKWMGWINTNTGEPTYAQ |
| | region | EFTGRFVFSLDTSVSTAYLQISSLKAEDTAV |
| | (VH) | YFCAPYDNFFAYWGQGTTVTVS |
| 21 | Light chain | DIVLTQSPLSLPVTPGEPASISCRSSKSLLY |
| | variable | KDGKTYLNWFLQKPGQSPQLLIYLMSTHASG |
| | region | VPDRFSGSGSGTDFTLKISRVEAEDVGVYYC |
| | (VH) | QQLVDYPLTFGGGTKLEIK |
| 22 | Heavy chain | QVQLVQSGSELKKPGASVKVSCKASGYTFTNY |
| | complete | GMNWVRQAPGQGLKWMGWINTNTGEPTYAQEF |
| | sequence | TGRFVFSLDTSVSTAYLQISSLKAEDTAVYFC |
| | | APYDNFFAYWGQGTTVTVSSASTKGPSVFPLA |
| | | PCSRSTSESTAALGCLVKDYFPEPVTVSWNSG |
| | | ALTSGVHTFPAVLQSSGLYSLSSVVTVPSSSL |
| | | GTKTYTCNVDHKPSNTKVDKRVESKYGPPCPP |
| | | CPAPEFLGGPSVFLFPPKPKDTLMISRTPEVT |
| | | CVVVDVSQEDPEVQFNWYVDGVEVHNAKTKPR |
| | | EEQFNSTYRVVSVLTVLHQDWLNGKEYKCKVS |
| | | NKGLPSSIEKTISKAKGQPREPQVYTLPPSQE |
| | | EMTKNQVSLTCLVKGFYPSDIAVEWESNGQPE |
| | | NNYKTTPPVLDSDGSFFLYSRLTVDKSRWQEG |
| | | NVFSCSVMHEALHNHYTQKSLSLSLG |
| 23 | Light chain | DIVLTQSPLSLPVTPGEPASISCRSSKSLLYKD |
| | complete | GKTYLNWFLQKPGQSPQLLIYLMSTHASGVPDR |
| | sequence | FSGSGSGTDFTLKISRVEAEDVGVYYCQQLVDY |
| | | PLTFGGGTKLEIKRTVAAPSVFIFPPSDEQLKS |
| | | GTASVVCLLNNFYPREAKVQWKVDNALQSGNSQ |
| | | ESVTEQDSKDSTYSLSSTLTLSKADYEKHKVYA |
| | | CEVTHQGLSSPVTKSFNRGEC |

An “anti-GAL3 antibody” that specifically binds to human GAL3 may be a chimeric antibody, a humanized antibody, a bispecific antibody, or an antigen-binding (i.e., a GAL-3 binding) fragment. The antigen-binding fragment may be an Fab, F(ab)₂, a scFv, a sc(Fv)₂, or a diabody. In some embodiments, the GAL3 antibody is an antagonistic antibody. An antagonistic anti-GAL3 antibody or antigen-binding fragment thereof antagonizes an activity of GAL3 (e.g., human GAL3) such as by inhibiting GAL3 binding to LAG3; competing with GAL3 for LAG3 binding; or when a cell or subject is contacted with the anti-GAL3 antibody or fragment thereof, biological activity associated with GAL3 antagonism occurs, such as proliferation of antigen-specific T-cells, CD107a degranulation, and/or stimulation of Th1-type cytokine production (e.g., interferon-γ (IFNγ) IL-2, IL-12, IL-18, IL-27, etc). In some embodiments, the GAL3 inhibitory agent is a polypeptide comprising soluble GAL3 or a polypeptide comprising soluble GAL3-immunoglobulin fusion. In some embodiments, the GAL3 inhibitory agent blocks the binding of GAL3 to carbohydrates on LAG3 (e.g. glycosylated sites).

In some embodiments, the antibody molecule comprises a heavy chain sequence having at least 70%, 80%, 85%, 90%, 95%, 96%, 97%, 98%, or 99% identity to SEQ ID NO: 22. In some embodiments, the antibody molecule comprises a light chain sequence having at least 70%, 80%, 85%, 90%, 95%, 96%, 97%, 98%, or 99% identity to SEQ ID NO: 23. In embodiments, the antibody molecule comprises a VH region having at least 70%, 80%, 85%, 90%, 95%, 96%, 97%, 98%, or 99% identity to SEQ ID NO: 20, or having the VH region from SEQ ID NO: 20. In embodiments, the antibody molecule comprises a VL region having at least 70%, 80%, 85%, 90%, 95%, 96%, 97%, 98%, or 99% identity to the VL region of SEQ ID NO: 21, or having the VL region of SEQ ID NO: 21. In embodiments, the antibody molecule comprises the HC CDR1, HC CDR2, and HC CDR3 of SEQ ID NOs: 14-16 and a LC CDR1, LC CDR2, and LC CDR3 of SEQ ID NOs: 17-19. In some instances, the antibody molecule comprises the HC CDR1, HC CDR2, and HC CDR3 of SEQ ID NO: 20 and the LC CDR1, LC CDR2, and LC CDR3 of SEQ ID NO:21. In some cases, the CDRs are defined according to Kabat, Chothia, enhanced Chothia, AbM, contact, IMGT, or Abysis definitions.

In some embodiments, the GAL3 inhibitory agent is a GAL3 inhibitor selected from any of the compounds disclosed in US Patent Nos. U.S. Ser. No. 10/774,102, U.S. Ser. No. 10/526,360, or U.S. Ser. No. 10/464,964; International Patent Application Publication Nos. WO2021004940, WO2021001538, WO2021001528, WO2020078808, WO2016004093, or WO2004091634; or US Patent Application Publication Nos. US20160122407, or US20160346317. In some embodiments, the GAL3 inhibitory agent is GCS-100, a citrus-derived polysaccharide inhibitor of GAL3. See e.g., Chauhan, D. et al, Cancer Res Sept 2005 65:18; 8350-8358.

In some embodiments, the GAL3 inhibitor is one of the agents disclosed in Table 3, as disclosed in Chan, Y. C. et al., Int. J. Mol. Sci. (2018), 19, 392.

TABLE 3
Galectin-3 inhibitory agents

| Galectin-3 inhibitors | Structure |
|---|---|
| Davanat ™ (GM-CT-01) | 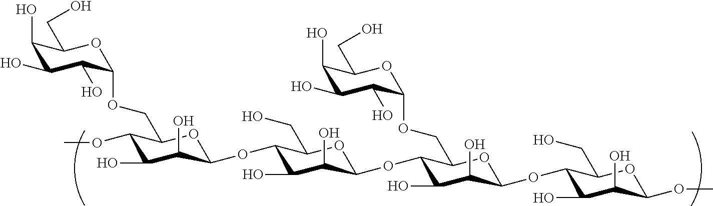 |
| Belapectin (GR-MD-02) | 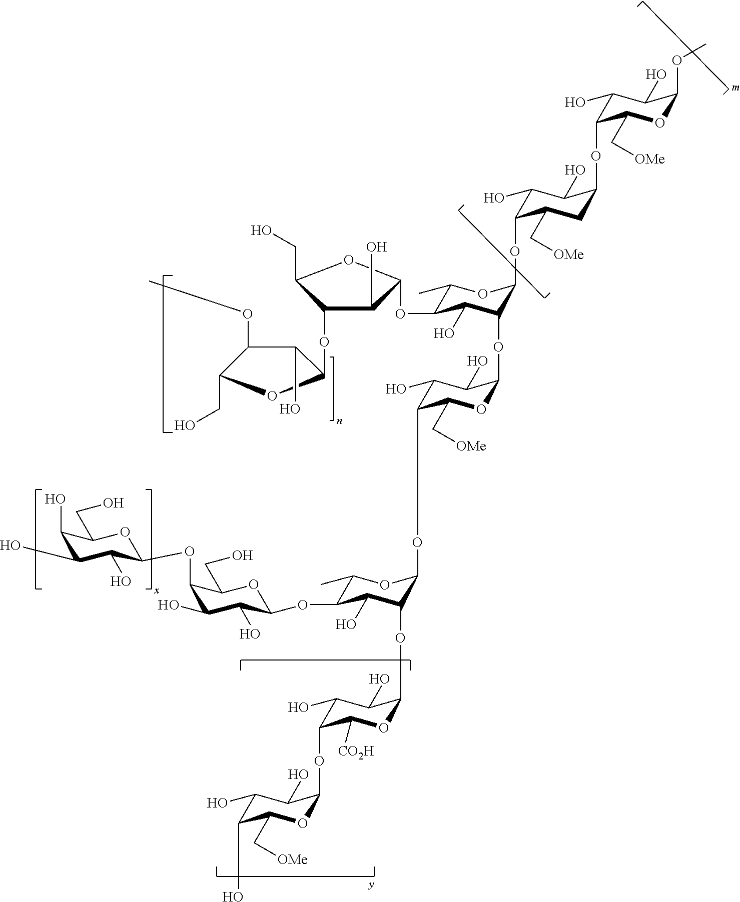 |
| GB0139 (TD139) | 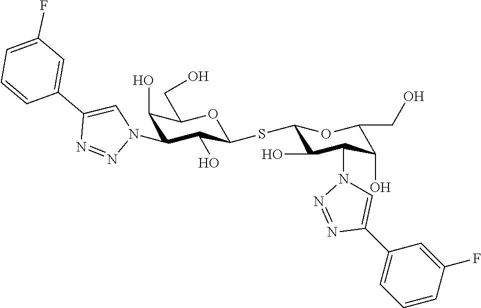 |

### HLA-A2 and HLA-A24 Immunogenic Peptides

The disclosure features isolated immunogenic peptides comprising an amino acid sequence that is identical to any one of SEQ ID NOs: 24-31, as depicted in Table 4 below, or differs from the sequences set forth below at 1, 2, 3, or 4 positions but still binds HLA-A2 or HLA-A24.

TABLE 4
HLA-A2+ and HLA-A24+ peptides

| SEQ | | | |
|---|---|---|---|
| ID | | | HLA- |
| NO: | Peptide | Sequence | restriction |
| 24 | non-spliced | YISPWILAV | HLA-A2+ |
| | XBP1 | | |
| 25 | spliced XBP1 | YLFPQLISV | HLA-A2+ |
| 26 | CD138 | GLVGLIFAV | HLA-A2+ |
| 27 | CS-1 | SLFVLGLFL | HLA-A2+ |
| 28 | non-spliced | ISPWILAVL | HLA-A24+ |
| | XBP1 | | |
| 29 | spliced XBP1 | VYPEGPSSL | HLA-A24+ |
| 30 | CD138 | IFAVCLVGF | HLA-A24+ |
| 31 | CS-1 | LFVLGLFLW | HLA-A24+ |

Other examples of immunogenic XBP1/CD138 and/or CS1 peptides that can be used in the compositions and methods of the disclosure include, but are not limited to the peptides provided in U.S. Pat. No. 9,950,047, the contents of which are incorporated by reference in their entirety herein.

Preferably, the isolated immunogenic peptide is at least 9, 10, 11, 12, 13, 14, 15, 16, 17, 18, 19, 20, 25, 30, 35, 40, 45, or 50 amino acids in length (e.g., between 9 and 50 amino acids in length, e.g., 9-50, 9-45, 9-40, 9-45, 9-30, 9-25, 9-20, 9-15 amino acids in length) and comprises an amino acid sequence that is at least 70%, 75%, 80%, 85%, 90%, 95%, 96%, 97%, 98%, 99% identity or is identical to an amino acid sequence of SEQ ID NOs: 24-31. Other preferred peptides can be at least 9, 10, 11, 12, 13, 14, 15, 16, 17, 18, 19, 20, 25, 30, 35, 40, 45, or 50 amino acids in length (e.g., between 9 and 50 amino acids in length, e.g., 9-50, 9-45, 9-40, 9-45, 9-30, 9-25, 9-20, 9-15 amino acids in length) and comprise an amino acid sequence of SEQ ID NOs: 24-31, or an amino acid sequence with one, two, three or four substitutions of the amino acid sequence of SEQ ID NOs: 24-31. These immunogenic peptides bind HLA-A2 or HLA-A24.

Percent identity between two peptide sequences (e.g., a peptide of SEQ ID NOs: 24-31 and another amino acid sequence that may be at least 66% identical to the peptide) can be determined using a variety of algorithms and computer programs including, but not limited to, Clustal W (The European Bioinformatics Institute (EMBL-EBI), BLAST-Protein (National Center for Biotechnology Information (NCBI), United States National Institutes of Health), and PSAlign (University of Texas A&M; Sze et al. Journal of Computational Biology (2006) 13:309-319).

Variants of the peptides described herein can include forms of the peptides having: (i) not more than 4 (e.g., 3, 2, or 1) amino acid substitutions (e.g., conservative or non-conservative substitutions); (ii) terminal or internal deletions; or (iii) terminal or internal additions, all of which are elaborated on below. These variant immunogenic peptides bind HLA-A2 or HLA-A24. The peptides of the disclosure comprise, consist of, or consist essentially of, an amino acid sequence of any of SEQ ID NOs: 24-31 (as depicted in Table 4), but with not more than four (e.g., not more than three, not more than two, or not more than 1) amino acid substitutions. The substitutions can be, e.g., conservative or non-conservative.

Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The non-polar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine, and histidine. The negatively charged (acidic) amino acids include aspartic acid and glutamic acid. Any substitution of one member of the above-mentioned polar, basic or acidic groups by another member of the same group can be deemed a conservative substitution. By contrast, a non-conservative substitution is a substitution of one amino acid for another with dissimilar characteristics.

In some embodiments, one or more (e.g., one, two, three, four, or all five) of positions three, four, five, six, seven, and eight of any of the peptides are not substituted. In some embodiments, one or more of positions three, four, five, six, seven, and eight of any of the peptides are identical to the amino acids of the peptides in Table 4.

“Non-spliced XBP1” peptides refer to a peptide having an amino acid sequence of at least 5 (e.g., 5, 6, 7, 8, 9, 10, 11, 12, 13, 14, 15, 16, 17, 18, 19, 20, 21, 22, 23, 24, 55 25, 26, 27, 28, 29, 30, 31, 32, 33, 34, 35, 40, 45, or 50) consecutive amino acids from the non-spliced form of human XBP1 protein having 261 amino acids and the following sequence:

MVVVAAAPNPADGTPKVLLLSGQPASAAGAPAGQALPLMVPAQRGASPE AASGGLPQARKRQRLTHLSPEEKALRRKLKNRVAAQTARDRKKARMSELEQQV VDLEEENQKLLLENQLLREKTHGLVVENQELRQRLGMDALVAEEEAEAKGNEV RPVAGSAESAALRLRAPLQQVQAQLSPLQNISPWILAVLTLQIQSLISCWAFWTTW TQSCSSNALPQSLPAWRSSQRSTQKDPVPYQPPFLCQWGRHQPSWKPLMN (SEQ ID NO: 38; Genbank Accession No. NP 005071), and peptides having no more than one, two, three, four, five substitutions (e.g., conservative substitutions) of the amino acids derived from the amino acid sequence of SEQ ID NO: 38. The amino acid positions referred to in Table 4 are based on SEQ ID NO: 38.

“Spliced XBP1” peptides refer to a peptide having an amino acid sequence of at least 5 (e.g., 5, 6, 7, 8, 9, 10, 11, 12, 13, 14, 15, 16, 17, 18, 19, 20, 21, 22, 23, 24, 25, 26, 27, 28, 29, 31, 32, 33, 34, 35, 40, 45, or 50) consecutive amino acids from the spliced form of human XBP1 (XBP1 spliced) protein having 376 amino acids and the following sequence:

MVVVAAAPNPADGTPKVLLLSGQPASAAGAPAGQALPLMVPAQRGASPE AASGGLPQARKRQRLTHLSPEEKALRRKLKNRVAAQTARDRKKARMSELEQQV VDLEEENQKLLLENQLLREKTHGLVVENQELRQRLGMDALVAEEEAEAKGNEV RPVAGSAESAAGAGPVVTPPEHLPMDSGGIDSSDSESDILLGILDNLDPVMFFKCP SPEPASLEELPEVYPEGPSSLPASLSLSVGTSSAKLEAINELIRFDHIYTKPLVLEIPS ETESQANVVVKIEEAPLSPSENDHPEFIVSVKEEPVEDDLVPELGISNLLSSSHCPK PSSCLLDAYSDCGYGGSLSPFSDMSSLLGVNHSWEDTFANELFPQLISV (SEQ ID NO: 33; Genbank Accession No. NP 001073007), and peptides having no more than one, two, three, four, five substitutions (e.g., conservative substitutions) of the amino acids derived from the amino acid sequence of SEQ ID NO:33. The amino acid positions referred to in Table 4 are based on SEQ ID NO: 33.

“CD138” peptides refer to a peptide having an amino acid sequence of at least 5 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50) consecutive amino acids from the human CD138 protein having 310 amino acids and the following sequence: MRRAALWLWLCALALSLQPALPQIVATNLPPEDQDGSGDDSDNFSGSGAGALQD ITLSQQTPSTWKDTQLLTAIPTSPEPTGLEATAASTSTLPAGEGPKEGEAVVLPEVE PGLTAREQEATPRPRETTQLPTTHQASTTTATTAQEPATSHPHRDMQPGHHETSTP AGPSQADLHTPHTEDGGPSATERAAEDGASSQLPAAEGSGEQDFTFETSGENTAV VAVEPDRRNQSPVDQGATGASQGLLDRKEVLGGVIAGGLVGLIFAVCLVGFMLYR MKKKDEGSYSLEEPKQAN GGAYQKPTKQEEFYA (SEQ ID NO: 34; Genbank Accession No. NP 002988) and peptides having no more than one, two, three, four, five substitutions (e.g., conservative substitutions) of the amino acids derived from the amino acid sequence of SEQ ID NO: 34. The amino acid positions referred to in Table 4 are based on SEQ ID NO: 34.

“CS-1” peptides refer to a peptide having an amino acid sequence of at least 5 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50) consecutive amino acids from the human CS-1 protein having 335 amino acids and the following sequence: MAGSPTCLTLIYILWQLTGSAASGPVKELVGSVGGAVTFPLKSKVKQVDSIVWTF NTTPLVTIQPEGGTIIVTQNRNRERVDFPDGGYSLKLSKLKKNDSGIYYVGIYSSSL QQPSTQEYVLHVYEHLSKPKVTMGLQSNKNGTCVTNLTCCMEHGEEDVIYTWK ALGQAANESHNGSILPISWRWGESDMTFICVARNPVSRNFSSPILARKLCEGAAD DPDSSMVLLCLLLVPLLLSLFVLGLFLWFLKRERQEEYIEEKKRVDICRETPNICPH SGENTEYDTIPHTNRTILKEDPANTVYSTVEIPKKMENPHSLLTMPDTPRLFAYEN VI (SEQ ID NO: 35; Genbank Accession No. NP_067004) and peptides having no more than one, two, three, four, five substitutions (e.g., conservative substitutions) of the amino acids derived from the amino acid sequence of SEQ ID NO: 35. The amino acid positions referred to in Table 4 are based on SEQ ID NO: 35.

The peptides described herein can bind to a major histocompatibility complex (MHC) molecule (e.g., an MHC class I molecule or an MHC class II molecule). The “Major Histocompatibility Complex” or “MHC” is a cluster of genes that plays a role in control of the cellular interactions responsible for physiologic immune responses. In humans, the MHC is known as the human leukocyte antigen (HLA) complex (see, e.g., Paul et al., FUNDAMENTAL IMMUNOLOGY, 3rd Edition, Raven Press, New York, (1993) and Stites, et al., IMMUNOLOGY, 8th Edition, Lange Publishing, Los Altos, Calif. (1994)). HLA molecules are described in detail in U.S. Pat. No. 7,026,443, the entire disclosure of which is incorporated by reference in its entirety.

In some embodiments, the peptides of this disclosure, e.g., XBP1 peptides, CD138 peptides and CS-1 peptides, have affinity for MHC molecules, e.g., HLA-A molecules such as HLA-A2 and HLA-A24 as indicated in Table 1, elevated stability within the peptide binding cleft of the MHC molecules, e.g., HLA-A2 and HLA-A24, and the ability, when expressed on the surface of cell (e.g., a cancer cell) in the context of an MHC molecule, e.g., HLA-A2 or HLA-A24, to induce the activation and proliferation of T cells including, e.g., effector memory T cells and/or central memory T cells).

In some embodiments, the peptides can be modified (e.g., amino acids of the peptides can be substituted) in order to modulate (e.g., increase or decrease) one of more properties of the peptides. For example, one or more (e.g., two, three, or four) amino acids of one of the HLA-A2 or the HLA-A24 restricted peptides depicted in Table 1 can be substituted in order to increase the affinity of the peptide for an MHC molecule. In some embodiments, an amino acid of one of the peptides described herein (e.g., a T cell Receptor contacting amino acid residue of the peptide) can be modified in order to enhance a binding interaction between a T cell receptor and the peptide (in the context of an MHC molecule). Such modified peptides are often referred to as “altered peptide ligands.” (See, e.g., Kalergis et al. J Immunol. (2000) 165(1):280; Conlon et al. Science (2002) 1801; and International Publication No. WO02070003, the disclosure of each of which is incorporated by reference in their entirety). In some embodiments, the first, second, and/or ninth amino acids of the peptides in Table 1 can be modified such that they retain or enhance the binding or stability to HLA-A2 or HLA-A24. In some embodiments, the third, fourth, fifth, sixth, seventh, or eighth amino acids can be modified for recognition by T-cell receptors. Suitable methods for modifying the peptides as well as determining the effect of the modification are described in U.S. Pat. No. 9,950,047 and are described in, e.g., Collins et al. Immunological Reviews (1998) 163:151-160, and Bae J. et al. Clinical Cancer Res. (2004): 10: 7043-7052, the disclosures of which are incorporated by reference in their entirety.

In some embodiments, only one of the HLA-A2- or HLA-A24- restricted peptides disclosed above (e.g., a non-spliced XBP1 peptide, a spliced XBP1 peptide, a CD138 peptide, or a CS-1 peptide) are present in the compositions of this disclosure or used in the methods of this disclosure. In other embodiments, at least two of the HLA-A2- or HLA-A24-restricted peptides disclosed above are present in the compositions or used in the methods of this disclosure. In yet other embodiments, at least three of the HLA-A2- or HLA-A24-restricted peptides disclosed above are present in the compositions or used in the methods of this disclosure. In some embodiments, either all four of the HLA-A2- or all four of the HLA-A24-restricted peptides disclosed above are present in the compositions or used in the methods of this disclosure.

The present disclosure provides for various types of therapeutic applications including multipeptide vaccination using the XBP1/CD138 and/or CS1 peptides described above, in a human subject with blood cancer (e.g., MM) or a pre-cancerous condition (e.g., MGUS or SMM). In some instances, the multipeptide vaccine induces antigen-specific T lymphocytes which can kill cancer cells expressing one or more of XBP1, CS1, and CD138. In some instances, the multipeptide vaccine induces antigen-specific T lymphocytes which can kill cancerous or pre-cancerous cells (e.g., SMM, MGUS or MM cancer cells). In some cases, the T lymphocytes are CD8+ CTLs. The disclosure also provides for combinations of multipeptide vaccines with additional approaches, such as adoptive T cell therapy as described below.

As used herein, the term “heteroclitic” (e.g., a heteroclitic peptide) refers to a form of a peptide in which one or more amino acid residues have been modified from a wild-type or original sequence in order to produce a peptide that is more immunogenic than the corresponding peptide with wildtype sequence or original sequence.

### Multipeptide Vaccine

In some embodiments, the methods of this disclosure include administering to a subject with a blood cancer (e.g., MM), or a pre-cancerous condition (e.g., MGUS or SMM), LAG3 or GAL3 inhibitory agents with a multipeptide vaccine comprising a mixture of immunogenic peptides from one or more (1, 2, 3) of XBP1, CS1, and CD138 (SEQ ID NOs.: 24-27 or 28-31) that can induce antigen-specific T lymphocytes with “anti-cancer activity”, i.e., with the ability to induce precancerous or cancerous cell death. An antigen-specific T lymphocyte (i.e., an XBP1, CS1, and/or CD138-specific T lymphocyte) in the context of this disclosure can eliminate precancerous or cancerous cells that express one or more of XBP1, CS1, and CD138 on the cell surface. The cell death can occur via apoptotic or non-apoptotic pathways. Anti-cancer activity can be elicited by granule exocytosis i.e., perforin (PRF1) and granule-associated enzymes (granzymes; GZM)], or via the death ligand/death receptor system. See, e.g., Martínez-Lostao L et al., Clin Cancer Res; (2015) 21(22); 5047-56. The antigen-specific T lymphocytes (i.e., XBP1, CS1, and/or CD138-specific T cells) with anti-cancer activity include both CD8 T cells (cytotoxic T lymphocytes (CTLs)) and CD4 T cells.

A multipeptide vaccine of this disclosure can be used to boost endogenous immune response in the patients and can be prepared by any methods known in the art. It may be administered directly into the patient, intradermally, intramuscularly, subcutaneously, intraperitoneally. and intravenously, or applied ex vivo to cells derived from the patient, an HLA-matched donor, or a human cell line, which cells are subsequently administered to the patient, or used in vitro to select a subpopulation of immune cells derived from the patient, which are then re-administered to the patient.

The peptides of the multipeptide vaccine may be substantially pure, or combined with an immune-stimulating adjuvant (e.g., incomplete Freund's adjuvant (IFA), Polyinosinic-polycytidylic acid, or poly-L-lysine (poly-ICLC) or used in combination with immune-stimulatory cytokines, and/or be administered with a suitable delivery system, for example liposomes. The peptides of the multipeptide vaccine may each be conjugated to a suitable carrier such as a keyhole limpet haemocyanin (KLH) or mannan (see WO 95/18145). The peptides of the present disclosure (SEQ ID NOs: 24-31) are expected to stimulate CD4 or CD8 T cells. In one aspect, the vaccine of the disclosure comprises at least one peptide having the amino acid sequence set forth in SEQ ID NOs: 24-31. In some aspects, the vaccine of the disclosure comprises at least two HLA-A2+ peptides having the amino acid sequences set forth in SEQ ID NOs.: 24-27. In some aspects, the vaccine of the disclosure comprises at least two HLA-A24+ peptides having the amino acid sequences set forth in SEQ ID NOs: 28-31. In some aspects, the vaccine of the disclosure comprises at least three HLA-A2+ peptides having the amino acid sequences set forth in SEQ ID NOs: 24-27. In some aspects, the vaccine of the disclosure comprises at least three HLA-A24+ peptides having the amino acid sequences set forth in SEQ ID NOs: 28-31. In some aspects, the vaccine of the disclosure comprises the four HLA-A2+ peptides having the amino acid sequences set forth in SEQ ID NOs: 24-27. In some aspects, the vaccine of the disclosure comprises the four HLA-A24+ peptides having the amino acid sequences set forth in SEQ ID NOs: 28-31.

### Nanoparticle-Based Immunotherapy

The present disclosure also provides for nanoparticle-based immunotherapy wherein the one or more peptides of this disclosure can be combined with nanoparticles (e.g., polymeric nanocarriers or liposomal nanoparticles). For instance, the one or more peptides (e.g., XBP1, CD138, and/or CS1 peptides) is encapsulated in the nanoparticle. In some embodiments, the nanoparticle is a liposome. In some embodiments, the nanoparticle comprises a biodegradable polymer. In some embodiments, the nanoparticle comprises poly(D,L-lactide-co-glycolide) (PLGA). In some embodiments, the nanoparticle comprises poly(lactic-co-glycolic acid)-poly(ethylene glycol) (PLGA-PEG) copolymer. The nanoparticles or nanocarriers can be administered to a subject (e.g., human) in need thereof to induce immune response.

The peptides can be attached to the nanoparticles or nanocarriers via various attachment mechanisms. This attachment mechanism can be an electrostatic attraction, covalent coupling, or a hydrophobic interaction. In some embodiments, the nanoparticles can be loaded with adjuvants. The adjuvants can be a dendritic cell targeting molecule, for example, a Toll-like receptor agonist, e.g., R-848, which is recognized as a potent synthetic agonist of TLR7/TLR8, or an unmethylated CpG oligodeoxynucleotide, which is immunostimulatory agonist of TLR-9, or monophosphoryl lipid A, which is immunostimulatory agonist of TLR-4, or an endosomal membrane targeting agent, e.g., the Endo-Porter peptide.

The polymer that forms the nanoparticles can be any biodegradable or non-biodegradable synthetic or natural polymer. Preferably, the polymer is a biodegradable polymer. Examples of useful biodegradable polymers include polylactic acid (PLA), poly(glycolic acid) (PGA), or poly(lactic-co-glycolic acid) (PLGA). These polymers have an established safety record and can be used in human subjects (Jiang, et al., Adv. Drug Deliv. Rev., 57(3): 391-410, 2005; Aguado and Lambert, Immunobiology, 184(2-3): 113-25, 1992; Bramwell, et al., Adv. Drug Deliv. Rev., 57(9): 1247-65, 2005). Other amphiphilic poly(amino acid) nanoparticles, amphiphilic polysaccharide nanoparticles, or polyion nanoparticles can be used in the vaccine composition disclosed herein (see, Akagi et al., Adv Polym Sci. 247: 31-64, 2012). The foregoing polymers can be used alone, as physical mixtures, or by forming copolymers. In certain embodiments, the nanoparticles are formed by a mixture of poly(lactic-co-glycolic acid)-block-poly(L-histidine)-block-poly(ethylene glycol) (PLGA-PLH-PEG)triblock copolymer; PLGA-PEG diblock copolymer, and PLA. These copolymers can be synthesized using standard techniques. For example, the copolymer PLGA-PLH-PEG can be synthesized using a block end-grafting strategy.

As used herein, a “nanoparticle” is a particle in the range of between 500 nm to 0.5 nm, e.g., having a diameter that is between 50 and 500 nm, having a diameter that is between 100 and 400 nm, or having a diameter that is between 200 and 400 nm. Nanoparticles and how to make and use nanoparticles are known in the art, and are described, e.g., in US 2016/0008451, US 2010/0129439, US2018/0021258, each of which is incorporated herein by reference in its entirety. Bae et al. describe using peptide engineered nanoparticles to enhance induction and function of antigen-specific T cells against multiple myeloma. See Bae J et al., Leukemia (2020): January 34(1): 210-223, which is incorporated herein by reference in its entirety. In some embodiments, the nanoparticle is a liposome. In some embodiments, the nanoparticle is a polymeric particle.

The polymer that forms the nanoparticles can be any biodegradable or non-biodegradable synthetic or natural polymer. In some embodiments, the polymer is a biodegradable polymer. Examples of useful biodegradable polymers include polylactic acid (PLA), poly(glycolic acid) (PGA), or poly(lactic-co-glycolic acid) (PLGA). These polymers have an established safety record and can be used in human subjects (Jiang, et al, Adv. Drug Deliv. Rev., 57(3):391-410, 2005; Aguado and Lambert, Immunobiology, 184(2-3): 113-25, 1992; Bramwell, et al., Adv. Drug Deliv. Rev., 57(9): 1247-65, 2005). Other amphiphilic poly(amino acid) nanoparticles, amphiphilic polysaccharide nanoparticles, or polyion nanoparticles can be used in the composition disclosed herein (see, Akagi et al, Adv Polym Sci. 247:31-64, 2012).

The polymers can be used alone, as physical mixtures, or by forming copolymers. In some embodiments, the nanoparticles are formed by a mixture of poly(lactic-co-glycolic acid)-block-poly(L-histidine)-block-poly(ethylene glycol) (PLGA-PLH-PEG)triblock copolymer; PLGA-PEG diblock copolymer, and PLA. These copolymers can be synthesized using standard techniques. For example, the copolymer PLGA-PLH-PEG can be synthesized using a block end-grafting strategy. A linear structure (e.g., PLGA-PLH-PEG) can provide the nanoparticles several characteristics compatible with extended circulation and charge-mediated targeting.

In some embodiments, natural polymers can be used. Examples of natural polymers include alginate and other polysaccharides, collagen, albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Other suitable biodegradable polymers include, but are not limited to, poly(hydroxy acids), such as polymers and copolymers of lactic acid and glycolic acid, polyanhydrides, poly(ortho)esters, polyesters, polyurethanes, poly(butic acid), poly(valeric acid), poly(caprolactone), poly(hydroxyalkanoates), and poly(lactide-co-caprolactone).

The polymer can be a bioadhesive polymer that is hydrophilic or hydrophobic. Hydrophilic polymers include CARBOPOL™ (a high molecular weight, crosslinked, acrylic acid-based polymers manufactured by Noveon), polycarbophil, cellulose esters, and dextran.

These polymers can be obtained from sources such as Sigma Chemical Co., St. Louis, Mo.; Polysciences, Warrenton, Pa.; Aldrich, Milwaukee, Wis.; Fluka, Ronkonkoma, N.Y.; and BioRad, Richmond, Calif, or can be synthesized from monomers obtained from these or other suppliers using standard techniques.

A wide variety of polymers and methods for forming polymeric matrices therefrom are known conventionally. In general, a polymeric matrix comprises one or more polymers. Polymers can be natural or unnatural (synthetic) polymers. Polymers can be homopolymers or copolymers comprising two or more monomers. In terms of sequence, copolymers can be random, block, or comprise a combination of random and block sequences. Typically, polymers in accordance with the present invention are organic polymers.

Examples of polymers suitable for use in the composition described herein include, but are not limited to polyethylenes, polycarbonates (e.g. poly(1,3-dioxan-2one)), polyanhydrides (e.g. poly(sebacic anhydride)), polypropylfumarates, polyamides (e.g., polycaprolactam), polyacetals, polyethers, polyesters (e.g., polylactide, polyglycolide, polylactide-co-glycolide, polycaprolactone, polyhydroxyacid (e.g. poly(-hydroxyalkanoate))), poly(orthoesters), polycyanoacrylates, polyvinyl alcohols, polyurethanes, polyphosphazenes, polyacrylates, polymethacrylates, polyureas, polystyrenes, and polyamines, polylysine, polylysine-PEG copolymers, and poly(ethyleneimine), poly(ethylene imine)-PEG copolymers.

In some embodiments, polymers in accordance with the present invention include polymers that have been approved for use in humans by the U.S. Food and Drug Administration (FDA) under 21 C.F.R. § 177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers can comprise anionic groups (e.g., phosphate group, sulfate group, carboxylate group); cationic groups (e.g., quaternary amine group); or polar groups (e.g., hydroxyl group, thiol group, amine group). In some embodiments, polymers can be hydrophobic. Selection of the hydrophilicity or hydrophobicity of the polymer can have an impact on the nature of materials that are incorporated (e.g., coupled) within the synthetic nanoparticle.

In some embodiments, polymers can be modified with one or more moieties and/or functional groups. A variety of moieties or functional groups can be used in accordance with the present invention. In some embodiments, polymers can be modified with polyethylene glycol (PEG), with a carbohydrate, and/or with acyclic polyacetals derived from polysaccharides (Papisov, 2001, ACS Symposium Series, 786:301). Certain embodiments can be made using the general teachings of U.S. Pat. No. 5,543,158 to Gref et al, or WO publication WO2009/051837 by Von Andrian et al.

In some embodiments, polymers can be modified with a lipid or fatty acid group. In some embodiments, a fatty acid group can be one or more of butyric, caproic, caprylic, capric, lauric, myristic, palmitic, stearic, arachidic, behenic, or lignoceric acid. In some embodiments, a fatty acid group can be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers can be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, poly(caprolactone), poly(caprolactone)-PEG copolymers, poly(L-lactide-co-L-lysine), poly(serine ester), poly(4-hydroxy-L-proline ester), poly[a-(4-aminobutyl)-L-glycolic acid], and derivatives thereof. The degradation rate of PLGA can be adjusted by altering the lactic acid: glycolic acid ratio. In some embodiments, PLGA to be used in accordance with the present invention is characterized by a lactic acid: glycolic acid ratio of approximately 85:15, approximately 75:25, approximately 60:40, approximately 50:50, approximately 40:60, approximately 25:75, or approximately 15:85.

In some embodiments, polymers can be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer can comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g., DNA, or derivatives thereof). Amine-containing polymers such as poly(lysine) (Zauner et al., 1998, Adv. Drug Del. Rev., 30:97; and Kabanov et al, 1995, Bioconjugate Chem., 6:7), poly(ethylene imine) (PEI; Boussif et al, 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al, 1993, Bioconjugate Chem., 4:372) are positively-charged at physiological pH, form ion pairs with nucleic acids, and mediate transfection in a variety of cell lines. In some embodiments, polymers can be degradable polyesters bearing cationic side chains (Putnam et al, 1999, Macromolecules, 32:3658; Barrera et al, 1993, J. Am. Chem. Soc, 115: 11010; Kwon et al, 1989, Macromolecules, 22:3250; Lim et al, 1999, J. Am. Chem. Soc, 121:5633; and Zhou et al., 1990, Macromolecules, 23:3399). Examples of these polyesters include poly(L-lactide-co-L-lysine) (Barrera et al, 1993, J. Am. Chem. Soc, 115: 11010), poly(serine ester) (Zhou et al, 1990, Macromolecules, 23:3399), poly(4-hydroxy-L-proline ester) (Putnam et al, 1999, Macromolecules, 32:3658; and Lim et al, 1999, J. Am. Chem. Soc, 121:5633), and poly(4-hydroxy-L-proline ester) (Putnam et al, 1999, Macromolecules, 32:3658; and Lim et al, 1999, J. Am. Chem. Soc, 121 :5633).

The properties of these and other polymers and methods for preparing them are well known in the art (see, for example, U.S. Patents 6, 123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al, 2001, J. Am. Chem. Soc, 123:9480; Lim et al, 2001, J. Am. Chem. Soc, 123:2460; Langer, 2000, Acc. Chem. Res., 33:94; Langer, 1999, J. Control. Release, 62:7; and Uhrich et al, 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al, Prentice-Hall, 1981; Deming et al., 1997, Nature, 390:386; and in U.S. Pat. Nos. 6,506,577, 6,632,922, 6,686,446, and 6,818,732. Each of the forgoing is incorporated herein by reference in its entirety.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanoparticles can comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, synthetic nanoparticles can optionally comprise one or more amphiphilic entities. In some embodiments, an amphiphilic entity can promote the production of synthetic nanoparticles with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanoparticles in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol;sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component can be a mixture of different amphiphilic entities. Those skilled in the art will recognize that this is an exemplary, not comprehensive, list of substances with surfactant activity. Any amphiphilic entity can be used in the production of synthetic nanoparticles to be used in accordance with the present invention.

In some embodiments, synthetic nanoparticles can optionally comprise one or more carbohydrates. Carbohydrates can be natural or synthetic. A carbohydrate can be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, hydroxyethylstarch, carageenan, glycon, amylose, chitosan, N,O-carboxylmethylchitosan, algin and alginic acid, starch, chitin, inulin, konjac, glucommannan, pustulan, heparin, hyaluronic acid, curdlan, and xanthan. In embodiments, the inventive synthetic nanoparticles do not comprise (or specifically exclude) carbohydrates, such as a polysaccharide. In certain embodiments, the carbohydrate can comprise a carbohydrate derivative such as a sugar alcohol, including but not limited to mannitol, sorbitol, xylitol, erythritol, maltitol, and lactitol.

The nanoparticles disclosed herein (e.g., liposomes or poly(lactic-co-glycolic acid) (PLGA) encapsulating one or more of the peptides disclosed herein) can be administered to a human subject in combination with LAG3 and/or GAL3 inhibitory agents, as well as another therapy described herein (e.g., multipeptide vaccine, ex vivo cellular immunotherapy). In some instances, a further therapeutic agent, such as an immune agonist (e.g., anti-OX40 antibody; anti-GITR antibody), and/or lenalidomide can be administered.

### Ex Vivo Cellular Immunotherapy

“Ex vivo cellular immunotherapy” in the context of this disclosure refers to the ex vivo process which involves harvesting cells from patients or donors, in vitro manipulation to enhance the therapeutic potential of the cell harvest, and subsequent administering (e.g., via intravenous transfusion) to the patients. The cells harvested by this process are referred to herein as “ex vivo activated immune cells”, which include ex vivo T cells (e.g., cytotoxic T lymphocytes of CTLs) and/or ex vivo PBMCs (e.g., dendritic cells) activated with the XBP1, CS1, and/or CD138 peptides of this disclosure.

In some embodiments, the methods of this disclosure include administering to a subject with a cancer (e.g., a blood cancer such as MM, leukemia e.g., (AML), or NHL), or a pre-cancerous condition (e.g., MGUS or SMM), ex vivo activated XBP1, CS1, and/or CD138 specific immune cells (e.g., antigen-specific T cells or PBMCs) with anti-cancer activity. The immune cells can be T cells or peripheral blood mononuclear cells (e.g., antigen presenting cells). The antigen-specific cytotoxic T lymphocytes (CTLs) of the disclosure that are generated ex vivo, have functional activity against HLA-A2+ or HLA-A24+MM cells. In some aspects, antigen-specific T cells can be generated with (a) peptide-stimulated T cell-based therapy; (b) PBMC-based therapy (e.g., dendritic cell (DC)-based therapy); (c) induced pluripotent stem cells (iPSC)-based therapy; or (d) T cell receptor (TCR)-based therapy. These are described in greater detail below.

### (a) T Cell-Based Immunotherapy

In peptide-stimulated T cell-based therapy (adoptive T cell therapy), the T cells or PBMC are generated ex vivo by stimulation with one or more (1, 2, 3, 4) of the XBP1, CD138 and/or CS1 peptide(s) (one or more of the peptides of SEQ ID NOs: 24-27 or one or more of the peptides of SEQ ID NOs.: 28-31) and the antigen-specific T cells induced can be used as adoptive therapy in combination with LAG3 and/or GAL3 inhibitory agents (and/or one or more of anti-OX40, anti-GITR, anti-PD1, anti-PD-L1 antibodies or antigen-binding fragments thereof) in human subjects with a cancer (e.g., a blood cancer such as MM), or a pre-cancerous condition (e.g., MGUS or SMM).

The method for generating adoptive T cells includes contacting in vitro a T cell (e.g., in a population of lymphocytes obtained from a subject) with an antigen-presenting cell (APC) expressing an MHC molecule bound to one or more of the peptides described herein for an amount of time (and under conditions) that is sufficient to activate the T cell (e.g., cytotoxic T cells and/or CD4+ helper T cells). Thus, the disclosure provides methods of generating and/or proliferating XBP1, CS1, and/or CD138 specific T cells (e.g., cytotoxic T cells and/or CD4+ helper T cells). The methods involve contacting one or more T cells (e.g., cytotoxic T cells and/or CD4+ helper T cells) with one or more antigen presenting cells pulsed with a peptide as described herein. These T cells can be cytotoxic T cells, e.g., memory cytotoxic T cells, effector cytotoxic T cells, or CD4+ helper T cells.

The activated T cells can be used to kill a target cell. In some embodiments, the methods involve contacting the target cell with cytotoxic T cells that are specific for one or more (e.g., 1, 2, 3) of the following: XBP1, CS1, and/or CD138, wherein the target cell expresses or overexpresses XBP1, CS1, and/or CD138, and expresses HLA-A2 or HLA-A24.

The activated T cells can also be reintroduced into the subject from which the cells were obtained. In some embodiments, T cells can be obtained from a subject of the same species other than the subject (allogeneic) can be contacted with the reagents (or immunogenic/antigenic compositions) and administered to the subject.

In some embodiments, T cells are derived from in vitro induction in patient-derived peripheral blood mononuclear cells (PBMC). The following protocol can be used to produce antigen specific CTL in vitro from patient derived PBMC. To generate dendritic cells, the plastic adherent cells from PBMCs are cultured in AIM-V medium supplemented with recombinant human GM-CSF and recombinant human IL-4 at 37° C. in a humidified CO₂ (5%) incubator. Six days later, the immature dendritic cells in the cultures are stimulated with recombinant human TNF-α for maturation. Mature dendritic cells are then harvested on day 8, resuspended in PBS at 1×10⁶ per mL with peptide (2 μg/mL), and incubated for 2 hours at 37° C. Autologous CD8+ T cells are enriched from PBMCs using magnetic microbeads (Miltenyi Biotech, Auburn, Calif.). CD8+ T cells (2×10⁶ per well) are cocultured with 2×10⁵ per well peptide-pulsed dendritic cells in 2 mL/well of AIM-V medium supplemented with 5% human AB serum and 10 units/mL rhIL-7 (Cell Sciences) in each well of 24-well tissue culture plates. About 20 U/ml of IL-2 is added 24 h later at regular intervals, 2 days after each restimulation. On day 7, lymphocytes are restimulated with autologous dendritic cells pulsed with peptide in AIM-V medium supplemented with 5% human AB serum, rhIL-2, and rhIL-7 (10 units/mL each). About 20 U/ml of IL-2 is added 24 h later at regular intervals, 2 days after each restimulation. On the seventh day, after the three rounds of restimulation, cells are harvested and tested the activity of CTL. The stimulated CD8+ cultured cells (CTL) are co-cultured with T2 cells (a human TAP-deficient cell line) pulsed with 2 μg/ml Her-2, gp100, AIM-2, MAGE-1, or IL13 receptor α2 peptides. After 24 hours incubation, IFN-γ in the medium is measured by ELISA assay.

### (b) Peripheral Blood Mononuclear Cells (PBMC)-Based Immunotherapy

In PBMC-based immunotherapy, PBMCs (e.g., antigen-presenting cells) are pulsed with XBP1, CD138, and/or CS1 peptide(s) (one or more of the peptides of SEQ ID NOs: 24-27 or one or more of the 28-31) and injected to patients in order to generate the antigen-specific T cells (i.e., T cells specific for XBP1, CD138, and/or CS1), and administered LAG3 and/or GAL-3 inhibitory agents.

An ex vivo strategy for inducing an immune response in a subject can involve contacting suitable PBMCs, in particular, antigen presenting cells (APCs; e.g., dendritic cells, monocytes, or macrophages) obtained from the subject with any of the peptides described herein. Alternatively, the cells can be transfected with a nucleic acid (e.g., an expression vector) encoding one or more of the peptides and optionally cultured for a period of time and under conditions that permit the expression of the peptides. The transfection method will depend on the type of cell and nucleic acid being transfected into the cell. Following the contacting or transfection, the cells are then returned to the subject.

The cells can be any of a wide range of types expressing MHC class I or II molecules. For example, the cells can include macrophages, monocytes, dendritic cells, T cells (e.g., T helper cells, CD4+ cells, CD8+ cells, or cytotoxic T cells), or B cells.

Thus, the disclosure provides a composition comprising a PBMC (e.g., an APC), wherein the APC presents a peptide sequence on its surface, wherein the peptide sequence comprises at least one major histocompatibility complex (MHC) class I or class II peptide epitope of one or more of the XBP1, CS1, and/or CD138 peptides of SEQ ID NOs: 24-31 or variants thereof as described elsewhere. In some embodiments, the APC is a dendritic cell. In some embodiments, the MHC peptide epitope is MHC class I peptide epitope (e.g., HLA-A2 or HLA-A24 peptide epitope). In some embodiments, the APC acquires the peptide sequence in vitro by exposure to a synthetic peptide comprising the peptide sequence.

In some embodiments of any of the ex vivo methods, cells that are obtained from the subject, or from a subject of the same species other than the subject (allogeneic), e.g., an HLA-matched donor, can be contacted with the reagents (or immunogenic/antigenic compositions) and administered to the subject.

In some embodiments, the composition comprises at least 10⁴, 10⁵, 10⁶, 10⁷, 10⁸, or 10⁹ PBMCs (e.g., dendritic cells). In some embodiments, the composition comprises less than 10⁵, 10⁶, 10⁷, 10⁸, 10⁹, or 10¹⁰ PBMCs (e.g., dendritic cells).

### Isolation and Preparation of APCs

The APCs can be isolated and prepared by methods known in the art. See, e.g., U.S. Patent Application Publ. No. US20200352995, U.S. Pat. No. 5,643,786, O'Doherty, U. et al., J. Exp. Med., (1993) 178: 1067-1078; Young J W et al, J. Exp. Med., (1990) 171: 1315-1332,; Freudenthal P S, Proc. Nat. Acad. Sci. USA, (1990) 57: 7698-7702; Macatonia et al., Immunol., (1989) 67: 285-289,; Markowicz S, J. Clin. Invest., (1990) 85: 955-961; Mehta-Damani A. et al., J. Immunol., (1994) 153: 996-1003; and Thomas R. et al., J. Immunol., (1993) 151: 6840-6852.

Briefly, APCs, such as dendritic cells (DC), suitable for administration to subjects (e.g., multiple myeloma patients) can be isolated or obtained from any tissue in which such cells are found, or can be otherwise cultured and provided. APC (e.g., DC) can be found, by way of example, in the bone marrow or peripheral blood mononuclear cells (PBMC) of a mammal, in the spleen of a mammal or in the skin of a mammal (i.e., Langerhan's cells, which possess certain qualities similar to that of DC, can be found in the skin). For instance, bone marrow can be harvested from a mammal and cultured in a medium that promotes the growth of DC. GM-CSF, IL-4 and/or other cytokines (e.g., TNF-α), growth factors and supplements can be included in this medium. After a suitable amount of time in culture in medium containing appropriate cytokines (e.g., suitable to expand and differentiate the DCs into mature DCs, e.g., 4, 6, 8, 10, 12, or 14 days), clusters of DC cultured in the presence of antigens of interest (e.g., in the presence of one or more HLA-A2+ peptides of SEQ ID NOs: 24-27 or one of more HLA-A24+ peptides of SEQ ID NOs: 28-31) and harvested using standard techniques. Antigens (e.g., peptides of the disclosure) can be added to cultures at a concentration of 1 μg/ml-50 μg/ml per antigen, e.g., 2, 5, 10, 20, 30, or 40 μg/ml per antigen.

In some embodiments, APC are isolated from a subject (e.g., a human). Mononuclear cells are isolated from blood using leukapheresis (e.g., using a COBE Spectra Apheresis System). The mononuclear cells are allowed to become adherent by incubation in tissue culture flasks for 2 hours at 37° C. Non-adherent cells are removed by washing. Adherent cells are cultured in medium supplemented with granulocyte macrophage colony stimulating factor (GM-CSF) (800 units/ml, clinical grade, Immunex, Seattle, Wash.) and interleukin-4 (IL-4)(500 units/ml, R&D Systems, Minneapolis, Minn.) for five days. On day five, TNF-α is added to the culture medium for another 3-4 days. On day 8 or 9, cells are harvested and washed, and incubated with peptide antigens for 16-20 hours on a tissue rotator. Peptide antigens are added to the cultures at a concentration of 10 μg/ml (per antigen).

Various other methods can be used to isolate the APCs, as would be recognized by one of skill in the art. DCs occur in low numbers in all tissues in which they reside, making isolation and enrichment of DCs a requirement. Any of a number of procedures entailing repetitive density gradient separation, fluorescence activated cell sorting techniques, positive selection, negative selection, or a combination thereof are routinely used to obtain enriched populations of isolated DCs.

The dendritic cells prepared according to methods described herein present epitopes corresponding to the antigens at a higher average density than epitopes present on dendritic cells exposed to a tumor cells (e.g., a multiple myeloma cells). The relative density of one or more antigens on antigen presenting cells can be determined by both indirect and direct means. Primary immune response of naïve animals is roughly proportional to antigen density of antigen presenting cells (Bullock TNJ et al., J. Immunol., (2003) 170: 1822-1829). Relative antigen density between two populations of antigen presenting cells can therefore be estimated by immunizing an animal with each population, isolating B or T cells, and monitoring the specific immune response against the specific antigen by, e.g., tetramer assays, ELISPOT, or quantitative PCR.

Relative antigen density can also be measured directly. In one method, the antigen presenting cells are stained with an antibody that binds specifically to the MHC-antigen complex, and the cells are then analyzed to determine the relative amount of antibody binding to each cell (see, e.g., Gonzalez P A et al., Proc. Natl. Acad. Sci. USA, (2005) 102: 4824-4829). Exemplary methods to analyze antibody binding include flow cytometry and fluorescence activated cell sorting. The results of the analysis can be reported e.g., as the proportion of cells that are positive for staining for an individual MHC-antigen complex or the average relative amount of staining per cell. In some embodiments, a histogram of relative amount of staining per cell can be created.

In some embodiments, antigen density can be measured directly by direct analysis of the peptides bound to MHC, e.g., by mass spectrometry (see, e.g., Purcell A W, Mol. Cell. Proteomics, (2004)3: 193-208). Typically, MHC-bound peptides are isolated by one of several methods. In one method, cell lysates of antigen presenting cells are analyzed, often following ultrafiltration to enrich for small peptides (see, e.g., Falk K et al., J. Exp. Med., (1991) 174: 425-434). In another method, MHC-bound peptides are isolated directly from the cell surface, e.g., by acid elution (see, e.g., Storkus W J et al., J. Immunother., (1993) 14: 94-103; Storkus et al., J. Immunol., (1993) 151: 3719-27). In another method, MHC-peptide complexes are immunoaffinity purified from antigen presenting cell lysates, and the MHC-bound peptides are then eluted by acid treatment (see, e.g., Falk K et al., Nature, 351: 290-296). Following isolation of MHC-bound peptides, the peptides are then analyzed by mass spectrometry, often following a separation step (e.g., liquid chromatography, capillary gel electrophoresis, or two-dimensional gel electrophoresis). The individual peptide antigens can be both identified and quantified using mass spectrometry to determine the relative average proportion of each antigen in a population of antigen presenting cells. In some methods, the relative amounts of a peptide in two populations of antigen presenting cells are compared using stable isotope labeling of one population, followed by mass spectrometry (see Lemmel C et al., Nat. Biotechnol., (2004) 22: 450-454).

### (c) Chimeric Antigen Receptor (CAR) T-Cell Based Immunotherapy

In T cell receptor (TCR)-based therapy, the TCR on XBP1/CD138/CS1 peptide(s)-specific T cells are transduced into T cells, which possess the characteristic of antigen-specific T cells, which cells can further be used as adoptive therapy in combination with LAG3 and/or GAL3 inhibitory agents. CAR-modified T cells can be engineered to target the MM-associated antigens (e.g., XBP1, CS1, and/or CD138; peptides of SEQ ID NOs: 24-27 or 28-31). T cells are genetically engineered to express CARs specifically directed towards antigens on the patient's cancer cells, which are then infused back into the patient.

The common form of CARs are fusions of single-chain variable fragments (scFv), fused to CD3-zeta transmembrane- and endodomain. The scFV can be derived from the antigen-specific receptor of T cells (e.g., XBP1, CS1, and CD138-specific cytotoxic T cells), or antibodies that specifically bind to the antigen.

In some embodiments, the sequence of the T cell receptors in XBP1, CS1, and CD138-specific cytotoxic T cells is determined, e.g., by sequencing. The sequence of the T cell receptors in XBP1, CS1, and CD138-specific cytotoxic T cells can be used to generate a CAR.

In some embodiments, these T cells are collected from the patient. In some embodiments, these T cells are obtained from induced pluripotent stem cell (iPSC).

Viral vectors such as retrovirus, lentivirus or transposon, are often used to integrate the transgene (e.g., CAR) into the host cell genome. Alternatively, non-integrating vectors such as plasmids or mRNA can be used to transfer the CAR gene to the T cells, and make T cells to express CAR under appropriate conditions.

### (d) Induced Pluripotent Stem Cell Immunotherapy

In iPSC-based therapy, the iPSCs are generated from the XBP1, CD138, and/or CS1 peptide(s) (peptides of SEQ ID NOs: 24-27 or 28-31)-specific T cells and then differentiated into rejuvenated antigen-specific T cells, which can be used as adoptive therapy in combination with LAG3 and/or GAL3 inhibitory agents.

The present disclosure also provides methods for using “induced pluripotent stem cells (iPSC)” to generate CTLs. iPSCs are a special type of pluripotent cell that are derived from adult somatic cells upon ectopic expression of a set of defined transcription factors. Importantly, tumor antigen-specific CTL can be reprogrammed by iPSC technology from antigen-specific CTL (Vizcardo et al. 2013, Ando M et al. Stem Cell Reports (2015), 5(4): 597-608). These iPSC-CTL are functionally rejuvenated and demonstrate longer telomeres (1.5 fold increase) and a higher proliferative capacity (5-50 fold increase) than the original CTL from which they were derived (Nishimura T et al. Stem Cell Cell (2013) 12(1): 114-126. This powerful reprogramming therapeutic approach has the potential to markedly increase the efficacy and durability of antigen-specific cancer immunotherapy. Thus, the disclosure provides methods of using rejuvenating cytotoxic T cells specific to XBP1, CS1, and/or CD138. In some embodiments, the methods of generating CTLs from iPSCs can increase the proliferative capacity by at least 5, 6, 7, 8, 9, 10, 20, 30, 40, 50, 60, 70, 80, 90, or 100 folds.

Activation of tumor-specific CTLs is the main goal of many cancer immunotherapies. The isolation of tumor-specific T-cells from a cancer patient, in vitro preparation (activation and expansion), and transfusion of these T-cells to the patient are basic steps of adaptive immunotherapy with T-cell. iPSC technology can be used to improve the efficacy of adoptive cell transfer immunotherapy (ACT).

The iPSC can be obtained from differentiated cells (e.g., fibroblasts, immune cells, T cells, B cells) induced through retroviral transfection of Yamanaka factors (a combination of Oct3/4, Sox2, Klf4, and c-Myc), and differentiated into T-cell lineages by culturing it on monolayer OP9-DL1 cell system in addition to Flt-3 ligand and IL-7.

In some embodiments, iPSCs can be generated from T-cells. After the expansion, these cells are differentiated again into T-cells. Human T lymphocyte can act as cell source for iPSC generation. Peripheral blood mononuclear cells (PBMCs) can be separated from whole blood by leukapheresis or venipuncture and then CD3+ T-cells can be expanded by stimulation with IL-2 and anti-CD3 antibody. T-cell-derived iPSCs (TiPS) can be generated from activated T-cell when exposed to retroviral transduction of the reprogramming factors. These T-iPSCs preserve their original T-cell receptor (TCR) gene rearrangements, so they can be used as an unlimited source of hematopoietic stem cells bearing endogenous tumor-specific TCR gene for cancer ACT therapy.

Thus, in some embodiments, iPSCs are generated from antigen-specific cytotoxic T cells. These antigen-specific T cells are generated by the methods as described herein, e.g., by contacting one or more T cells with one or more antigen presenting cells pulsed with a peptide comprising an amino acid sequence as described herein (e.g., SEQ ID NOs: 24-27 or SEQ ID NOs: 28-31). As the T-iPSCs preserve their original T-cell receptor (TCR) gene rearrangements, after these T-iPSCs differentiates into T cells, these T cells can recognize XBP1, CS1, and/or CD138 on a cancer cell (e.g., an MM cell).

In some embodiments, a nucleic acid that encodes CAR that specifically recognizes XBP1, CS1, and/or CD138 can be introduced into T-iPSCs. Once after these T-iPSCs differentiates into T cells, these T cells can recognize XBP1, CS1, and/or CD138 on a cancer cell (e.g., an MM cell).

In some embodiments, the differentiated T cells are administered to a subject. In some embodiments, T-iPSCs are administered to a subject, and then these cells are differentiated into cytotoxic T cells in the body of the subject.

### Additional Treatments

This disclosure features combination therapies wherein the LAG3 and/or GAL3 inhibitory agent is administered with the multipeptide vaccine or adoptive T cell therapy of this disclosure to a subject (e.g. a subject with MM) in combination with one or more additional treatments. The additional treatment can be an art-recognized therapy for blood cancers (e.g., MM, leukemia, or NHL) and/or a therapy that prevents the pre-cancerous blood condition from progressing into a blood cancer. See van de Donk NWCJ, et al., Lancet. (2021) Jan. 30; 397(10272):410-427 for a review of treatments that can be used for MM, SMM, and MGUS. Such treatments include, but are not limited to the following: one or more forms of ionizing radiation and/or one or more agents selected from the group consisting of a therapeutic antibody, an immunomodulatory drug, a histone deacetylase (HDAC) inhibitor, an antineoplastic agent, a proteasome inhibitor, an antibody-drug conjugate, a nuclear export inhibitor and a corticosteroid. The therapeutic antibody that can be used as an additional treatment includes, but is not limited to, an anti-PD1 antibody (e.g., Pembrolizumab, Nivolumab, or Dostarlimab), an anti-PD-L1 antibody (e.g., Durvalumab), an anti-CD38 antibody (e.g., Daratumumab or Isatuximab), an anti-SLAMF7 antibody (e.g., Elotuzumab), an anti-CTLA4 antibody (e.g., Ipilimumab or Tremelimumab), an anti-TIM3 antibody (e.g., Cobolimab), an anti-VISTA antibody (e.g., SG7 or W0180), an anti-OX-40 antibody (e.g., PF-04518600, or IBI101), or an anti-GITR antibody (e.g., BMS-986156). The immunomodulatory drug that can be used as an additional treatment includes, but is not limited to, lenalidomide, pomalidomide, or thalidomide. The HDAC inhibitor that can be used as an additional treatment includes, but is not limited to, citarinostat or panobinostat. The antineoplastic agent that can be used as an additional treatment includes, but is not limited to, cyclophosphamide, etoposide, oxorubicin, liposomal doxorubicin, melphalan, melphalan flufenamide, and bendamustine. The proteasome inhibitor that can be used as an additional treatment includes, but is not limited to, bortezomib, carfilzomib, and ixazomib. The antibody-drug conjugate that can be used as an additional treatment includes, but is not limited to, belantamab mafodotin-blmf. The nuclear export inhibitor that can be used as an additional treatment includes, but is not limited to, selinexor. The corticosteroid that can be used as an additional treatment includes, but is not limited to, dexamethasone or prednisone.

In some embodiments, the combination therapy disclosed herein includes an antibody or PD-1-binding fragment thereof comprising the six CDRs of the anti-PD1 antibody Pembrolizumab. The CDRS can bev based on any definition known in the art (e.g., Kabat, Chothia, enhanced Chothia, contact, Abysis, AbM, IMGT, or contact definitions). In some embodiments, the combination therapy disclosed herein includes an antibody or PD-1-binding fragment thereof comprising the VH and VL of the anti-PD1 antibody Pembrolizumab. In some embodiments, the combination therapy disclosed herein includes an antibody or PD-1-binding fragment thereof comprising the heavy and light chains of the anti-PD1 antibody Pembrolizumab. Pembrolizumab is also disclosed in U.S. Pat. No. 9,995,753, incorporated by reference in its entirety.

The International Nonproprietary Names for Pharmaceutical Substances (INN) (WHO Drug Information, Vol. 28, No. 3, 2014) (incorporated by reference in its entirety, including the section entitled “pembrolizumab” on p. 407) provides the pembrolizumab heavy and light chain sequences as:

Pembrolizumab Heavy chain sequence:
(SEQ ID NO: 36)
QVQLVQSGVE VKKPGASVKV SCKASGYTFT NYYMYWVRQA
PGQGLEWMGG INPSNGGTNF NEKFKNRVTL TTDSSTTTAY
MELKSLQFDD TAVYYCARRD YRFDMGFDYW GQGTTVTVSS
ASTKGPSVFP LAPCSRSTSE STAALGCLVK DYFPEPVTVS
WNSGALTSGV HTFPAVLQSS GLYSLSSVVT VPSSSLGTKT
YTCNVDHKPS NTKVDKRVES KYGPPCPPCP APEFLGGPSV
FLFPPKPKDT LMISRTPEVT CVVVDVSQED PEVQFNWYVD
GVEVHNAKTK PREEQFNSTY RVVSVLTVLH QDWLNGKEYK
CKVSNKGLPS SIEKTISKAK GQPREPQVYT LPPSQEEMTK
NQVSLTCLVK GFYPSDIAVE WESNGQPENN YKTTPPVLDS
DGSFFLYSRL TVDKSRWQEG NVFSCSVMHE ALHNHYTQKS
LSLSLGK
Pembrolizumab Light chain sequence:
(SEQ ID NO: 37)
EIVLTQSPAT LSLSPGERAT LSCRASKGVS TSGYSYLHWY
QQKPGQAPRL LIYLASYLES GVPARFSGSG SGTDFTLTIS
SLEPEDFAVY YCQHSRDLPL TFGGGTKVEI KRTVAAPSVF
IFPPSDEQLK SGTASVVCLL NNFYPREAKV QWKVDNALQS
GNSQESVTEQ DSKDSTYSLS STLTLSKADY EKHKVYACEV.

The components of the combination therapy may be administered substantially at the same time or sequentially.

### Pharmaceutical Compositions and Combinations

Any of the inhibitory agents, peptides (or nucleic acids encoding the peptides), cells, and additional therapeutic agents described herein can be incorporated into pharmaceutical compositions. The pharmaceutical compositions can include the individual agent (e.g., anti-LAG3 antibody or multipeptide vaccine) or a combination of the agents along with a pharmaceutically acceptable carrier. The first component can include a LAG3 inhibitory agent and/or a GAL3 inhibitory agent. The second component can include one or more of the HLA-A2+ or HLA-A24+ peptides (e.g., one or a mixture of two, three, or four of the XBP1, CD138 and/or CS1 peptides of SEQ ID NOs: 24-27 or SEQ ID NOs: 28-31). Preferably, the second component comprises two or more (e.g., 2, 3, or 4) of the peptides described herein (e.g., two or more of SEQ ID NOs 24-27 or SEQ ID NOs: 28-31) or nanoparticles containing those peptides. Alternately, the second component can include cells (e.g., ex vivo activated T cells or PBMCs that are specific for XBP1, CD138 and/or CS1 peptides). As used herein the language “pharmaceutically acceptable carrier” includes solvents, dispersion media, coatings, antibacterial and antifungal agents, isotonic and absorption delaying agents, and the like, compatible with pharmaceutical administration. One or more peptides or inhibitory agents can be formulated as a pharmaceutical composition in the form of a syrup, an elixir, a suspension, a powder, a granule, a tablet, a capsule, a lozenge, a troche, an aqueous solution, a cream, an ointment, a lotion, a gel, an emulsion, etc. Supplementary active compounds (e.g., one or more chemotherapeutic agents) can also be incorporated into the compositions.

A pharmaceutical composition is generally formulated to be compatible with its intended route of administration. Examples of routes of administration include oral, rectal, and parenteral, e.g., intravenous, intra-arterial, intranasal, intraperitoneal, intramuscular, intradermal, subcutaneous, inhalation, transdermal, or transmucosal. In some embodiments, the composition is administered instranasally. See, e.g., Kumar S, et al. Vaccine. 2017; 35(7):1080-1086. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The compositions can be enclosed in ampoules, disposable syringes or multiple dose vials made of glass or plastic.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ.) or phosphate buffered saline (PBS). In all cases, the pharmaceutical composition must be sterile and should be fluid to the extent that easy syringability exists. It should be stable under the conditions of manufacture and storage and must be preserved against any contamination by microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of contamination by microorganisms can be achieved by various antibacterial and antifungal agents, for example, parabens, chlorobutanol, phenol, ascorbic acid, thimerosal, and the like. In many cases, it will be desirable to include isotonic agents, for example, sugars, polyalcohols such as manitol, sorbitol, sodium chloride in the composition. Prolonged absorption of the injectable compositions can be facilitated by including in the composition an agent that delays absorption, for example, aluminum monostearate and gelatin.

Sterile injectable solutions can be prepared by incorporating one or more of the inhibitory agents or peptides (or one or more the nucleic acids encoding the peptides) in the required amount in an appropriate solvent with one or a combination of ingredients, as required, followed by filtered sterilization. Generally, dispersions are prepared by incorporating the inhibitory agents, peptide(s) (or nucleic acid(s) encoding the peptide(s)) into a sterile vehicle which contains a basic dispersion medium and the required other ingredients from those enumerated above. In the case of sterile powders for the preparation of sterile injectable solutions, the methods of preparation can include vacuum drying or freeze-drying which yields a powder of the active ingredient plus any additional desired ingredient from a previously sterile-filtered solution thereof.

Oral compositions generally include an inert diluent or an edible carrier. For the purpose of oral therapeutic administration, the one or more peptides can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

The powders and tablets can contain from 1% to 95% (w/w) of an individual peptide or a mixture of two or more peptides. In certain embodiments, the peptide can range from about 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the inhibitory agent, or peptide (or nucleic acid) with encapsulating material as a carrier providing a capsule in which the inhibitory agent, or peptide with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

For administration by inhalation, the inhibitory agent, peptides or nucleic acids can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the inhibitory agents, peptides or nucleic acids can be formulated into ointments, salves, gels, or creams as generally known in the art.

The inhibitory agents, peptides or nucleic acids can also be prepared in the form of suppositories (e.g., with conventional suppository bases such as cocoa butter and other glycerides) or retention enemas for rectal delivery.

In some embodiments, the inhibitory agents, peptides or nucleic acids can be prepared with carriers that will protect the inhibitory agents or peptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to, e.g., APCs with monoclonal antibodies to APC-specific antigens) can also be used as pharmaceutically acceptable carriers. These can be prepared according to methods known to those skilled in the art, for example, as described in U.S. Pat. No. 4,522,811.

It can be advantageous to formulate oral or parenteral compositions in dosage unit form for ease of administration and uniformity of dosage. Dosage unit form, as used herein, refers to physically discrete units suited as unitary dosages for the subject to be treated; each unit containing a predetermined quantity of the peptides (or nucleic acids) calculated to produce the desired therapeutic effect in association with the required pharmaceutical carrier. Dosage units can also be accompanied by instructions for use.

The nucleic acid molecules encoding the peptides can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al. Proc. Natl. Acad. Sci. USA (1994) 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Additional examples of gene delivery vehicles include, but are not limited to, liposomes, biocompatible polymers, including natural polymers and synthetic polymers; lipoproteins; polypeptides; polysaccharides; lipopolysaccharides; artificial viral envelopes; metal particles; bacteria; viruses such as baculovirus, adenovirus, and retrovirus; bacteriophage; cosmids; plasmids; fungal vectors and other recombination vehicles typically used in the art which have been described for expression in a variety of eukaryotic and prokaryotic hosts, and may be used for gene therapy as well as for simple protein expression.

Examples of viral vectors include retroviral vectors, lentivirus vectors, adenovirus vectors, adeno-associated virus vectors, alphavirus vectors and the like. Liposomes that comprise a targeting moiety such as an antibody or fragment thereof can also be used to prepare pharmaceutical compositions of nucleic acids for delivery to a subject. Any of the pharmaceutical compositions described herein can be included in a container, pack, or dispenser together with instructions for administration as described below.

### Administration of Multipeptide Vaccine

Generally, the multipeptide vaccine of the disclosure to be delivered to the subject will be suspended in a pharmaceutically-acceptable carrier (e.g., physiological saline) and administered orally, rectally, or parenterally, e.g., injected intravenously, subcutaneously, intramuscularly, intrathecally, intraperitoneally, intrarectally, intravaginally, intranasally, intragastrically, intratracheally, or intrapulmonarily. The multipeptide vaccine can be administered with any adjuvant.

Administration can be by periodic injections of a bolus of the pharmaceutical composition or can be uninterrupted or continuous by intravenous or intraperitoneal administration from a reservoir which is external (e.g., an IV bag) or internal (e.g., a bioerodable implant, a bioartificial organ, or a colony of implanted reagent production cells). See, e.g., U.S. Pat. Nos. 4,407,957, 5,798,113 and 5,800,828, each incorporated herein by reference in their entirety.

In general, the dosage of the multipeptide vaccine (or each of the peptides in the vaccine) required depends on the choice of the route of administration; the nature of the formulation; the nature or severity of the subject's illness; the immune status of the subject; the subject's size, weight, surface area, age, and sex; other drugs being administered; and the judgment of the attending medical professional.

In some embodiments, a multipeptide vaccine of the disclosure can be administered to a subject at least two (e.g., three, four, five, six, seven, eight, nine, 10, 11, 12, 15, or 20 or more) times. For example, the multipeptide vaccine can be administered to a subject once a month for three months; once a week for a month; every other week, once a year for three years, once a year for five years; once every five years; once every ten years; or once every three years for a lifetime.

### Administration of Ex Vivo Immune Cells

The ex vivo activated immune cells (antigen-specific T cells and/or PBMCs) of the disclosure may be delivered to a subject by any suitable delivery route, which can include injection, infusion, inoculation, direct surgical delivery, or any combination thereof. In some embodiments, the ex vivo activated immune cells are administered to a human in the deltoid region or axillary region. For example, the cells are administered into the axillary region as an intradermal injection. In some embodiments, the cells are administered intravenously.

An appropriate carrier for administering the cells may be selected by one of skill in the art by routine techniques. For example, the pharmaceutical carrier can be a buffered saline solution, e.g., cell culture media, and can include DMSO for preserving cell viability.

The quantity of cells appropriate for administration to a subject in need thereof to effect the methods of the present disclosure and the most convenient route of such administration may be based upon a variety of factors, as may the formulation of the cells themselves. Some of these factors include the physical characteristics of the subject (e.g., age, weight, and sex), the physical characteristics of the blood cancer or pre-cancerous condition, and the extent to which other therapeutic methodologies (e.g., chemotherapy or corticosteroids) are being implemented in connection with an overall treatment regimen. Notwithstanding the variety of factors one should consider in implementing the methods of the present disclosure to treat the blood cancer or pre-cancerous condition, a mammal can be administered with from about 10⁵ to about 10⁸ PBMCs (e.g., 10⁷ cells) in from about 0.05 mL to about 2 mL solution (e.g., saline) in a single administration. Additional administrations can be carried out, depending upon the above-described and other factors, such as the severity of the condition. In one embodiment, from about one to about five administrations of about 10⁶ cells is performed at two-week intervals.

In some aspects, the disclosure includes combinations of the therapeutic agents described herein. For instance, the disclosure contemplates a “combination” of a LAG3 and/or GAL3 inhibitory agent, and means for targeting HLA-A2+ or HLA-A24+ pre-cancerous or cancerous cells that express one or more of XBP1, CS1, and CD138. Such means for targeting include a multipeptide vaccine and/or ex vivo activated immune cells specific for 1, 2, 3, or 4 of the HLA-A2+ or the HLA-A24+ peptides disclosed herein. In some cases, the combination includes means for specifically binding human LAG3 and/or means for specifically binding human GAL3, and anti-myeloma-specific T lymphocytes or PBMCs targeting XBP1, CD138 and CS1-expressing cells from SMM, MGUS, or MM patients.

In some embodiments, the term “combination” implies administration of these therapeutic agents in a sequential manner, wherein each therapeutic agent is administered at a different time, as well as administration of these therapeutic agents, or at least two of the therapeutic agents concurrently, or in a substantially simultaneous manner. Simultaneous administration can be accomplished, for example, by administering to the subject a single capsule having a fixed ratio of each therapeutic agent or in multiple, single capsules for each of the therapeutic agents. Sequential, or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. Therapeutic agents may also be administered in alternation. In one example, where the combination therapy involves administering an anti-LAG3/GAL3 inhibitory agent with a multipeptide vaccine as well ex vivo activated immune cells of this disclosure, the inhibitory agent may be administered orally, the multipeptide vaccine may be administered subcutaneously, and the immune cells may be administered intravenously.

### Kits

A kit comprising (a) a first composition comprising at least three of a non-spliced XBP1 peptide, a spliced XBP1 peptide, a CD138 peptide and a CS-1 peptide (and/or ex vivo activated T cells specific to one or more (1, 2, 3, 4) of a non-spliced XBP1 peptide, a spliced XBP1 peptide, a CD138 peptide and a CS-1 peptide), (b) a second composition comprising an anti-LAG3 antibody and/or an anti-GAL3 antibody, and optionally, (c) instructions for administering the first and second compositions to a subject. In some cases, the kit can also comprise one or more of an anti-OX40 antibody or OX40-binding fragment thereof, an anti-PD-1 antibody or PD-1-binding fragment thereof, an anti-PD-L-1 antibody or PD-1-binding fragment thereof, or an anti-GITR antibody or GITR-binding fragment thereof,

The disclosure also features a variety of kits. The kits can include two compositions. The first composition can include e.g., one or more (e.g., one, two, three, or four) of any of the HLA-A2+ or HLAA24+ peptides (or expression vectors containing nucleic acid sequences encoding one or more peptides) described herein. The second composition can include e.g., a LAG3 and/or GAL3 inhibitory agent. The kits can further include instructions for administering the first and second compositions to the subject. The kits can include one or more pharmaceutically acceptable carriers and/or one or more immune stimulating agents. The immune stimulating agents can be, e.g., an adjuvant. The kits can also contain one or more additional therapeutic agents, including but not limited to: an anti-PD1 antibody (e.g., Pembrolizumab, Nivolumab, or Dostarlimab), an anti-PD-L1 antibody (e.g., Durvalumab), an anti-CD38 antibody (e.g., Daratumumab or Isatuximab), an anti-SLAMF7 antibody (e.g., Elotuzumab), an anti-CTLA4 antibody (e.g., Ipilimumab or Tremelimumab), an anti-TIM3 antibody (e.g., Cobolimab), an anti-VISTA antibody (e.g., SG7 or W0180), an anti-OX-40 antibody (e.g., PF-04518600, or IBI101), and an anti-GITR antibody (e.g., BMS-986156); an immunomodulatory drug (e.g., lenalidomide, pomalidomide, or thalidomide; an HDAC inhibitor (e.g., citarinostat or panobinostat); a antineoplastic agent (e.g., cyclophosphamide, etoposide, oxorubicin, liposomal doxorubicin, melphalan, melphalan flufenamide, and bendamustine; a proteasome inhibitor (e.g., bortezomib, carfilzomib, and ixazomib), an antibody-drug conjugate (e.g., belantamab mafodotin-blmf; a nuclear export inhibitor (e.g., selinexor); a corticosteroid (e.g., dexamethasone or prednisone).

In some embodiments, the kits can contain one or more of any of the anti-LAG3 or anti-GAL3 antibodies described herein. In some embodiments, the kits can include two antibodies, each specific for a different protein. For example, a kit can contain one LAG3-specific antibody (described herein) and one GAL3-specific antibody (described herein). The kits can optionally include instructions for assaying a biological sample for the presence or amount of one or more of LAG3 and/or GAL3 proteins. Also featured are articles of manufacture that include: a container; and a composition contained within the container, wherein the composition comprises an active ingredient for inducing an immune response in a mammal (e.g., a human), wherein the active ingredient comprises one or more (e.g., two, three, or four) of any of the peptides described herein, and wherein the container has a label indicating that the composition is for use in inducing an immune response in a mammal (e.g., any of the mammals described herein). The label can further indicate that the composition is to be administered to a mammal having, suspected of having, or at risk of developing, multiple myeloma. The composition of the article of manufacture can be dried or lyophilized and can include, e.g., one or more solutions (and/or instructions) for solubilizing a dried or lyophilized composition.

The articles of manufacture can also include instructions for administering the composition to the mammal.

### Methods of Treatment

The disclosure features a variety of methods for treating a cancer (e.g., blood cancer such as MM, leukemia, or NHL, or Waldenstrom's macroglobulinemia, or any other blood cancer expressing XBP1, CD138 and/or CS1 in a subject); and/or a pre-cancerous blood condition (e.g., smoldering multiple myeloma (SMM) or monoclonal gammopathy of undermined significance (MGUS)) using the combinations, compositions, pharmaceutical compositions, and kits described herein. A human subject at risk of developing blood cancer is said to have a “pre-cancerous blood condition”. In some embodiments, the disclosure also provides methods for preventing the development of blood cancer (e.g., MM) from a pre-cancerous condition (e.g., SMM or MGUS), i.e., the treated subject does not develop a clinically observable level of the blood cancer at all (e.g., the subject does not exhibit one or more symptoms of MM or the subject does not develop a detectable level of MM). In other embodiments, the disclosure provides methods for increasing T lymphocyte responses in a tumor microenvironment while reducing immunosuppression in a human subject.

As used herein, the term “treat” “treatment,” or “treating” a subject having blood cancer or a pre-cancerous condition, are used in connection with a given treatment for a given disorder, wherein at least one symptom of the disorder is alleviated, or ameliorated. The treatment may inhibit deterioration or worsening of a symptom of the disclosed conditions (e.g., MM, leukemia, or NHL) or may cause the condition to develop more slowly and/or to a lesser degree (e.g., fewer symptoms or lower numbers of cancer cells in the subject) in the subject than it would have absent the treatment. For example, a treatment will be said to have “treated” the condition if it is given during the condition, e.g., during an early diagnosis of a cancer (e.g., early MM), (e.g., the detection of a few cancer cells in a sample from the subject), or during the stage of the pre-cancerous condition (e.g., MGUS or SMM) that would have been expected to produce a given manifestation of the condition (e.g., progressive multiple myeloma), and results in the subject's experiencing fewer and/or milder symptoms of the condition than otherwise expected. A treatment can “treat” a cancer (e.g., MM, leukemia, NHL, or Waldenstrom's macroglobulinemia) when the subject displays only mild overt symptoms of the blood cancer.

In some embodiments, the methods disclosed herein extend the progression-free survival (PFS), objective response rate (ORR), and/or overall survival (OS) of a patient with a blood cancer (e.g., multiple myeloma). In some embodiments of the compositions or methods for use herein extend the time to next treatment (TTNT) for a patient.

In some embodiments of the compositions or methods for use herein increase the chance of achieving a negative minimal residual disease (MRD). Minimal residual disease (MRD) in multiple myeloma is an independent predictor of progression-free survival (PFS) and can be used as a trial endpoint to improve the identification of effective treatments in multiple myeloma. Monitoring minimal residual disease (MRD) in patients with multiple myeloma thus provides prognostic value in predicting PFS and OS and making treatment decisions. The detection of minimal residual disease (MRD) in myeloma can use a 0.01% threshold (10+) after treatment, i.e., having 10+ cells or fewer is considered MRD-negative, and having 10+ cells or higher MRD-positive. Rawstron et al, Blood. 2015; 125(12): 1932-1935 (2015). Methods for measuring MRD include polymerase chain reaction (PCR) and multiparameter flow cytometry (MPF). Assays for MRD, e.g., based on clonotype profile measurement are also described in U.S. Pat. No. 8,628,927, to Faham et al., which is incorporated herein by reference.

In some embodiments, the LAG3 and/or GAL3 inhibitory agent is administered to a subject (e.g. a subject with MM) as a monotherapy. In other embodiments, the LAG3 and/or GAL3 inhibitory agent is administered to a subject (e.g. a subject with MM) in combination with a multipeptide vaccine that includes the HLA-A2+ or HLA-A24+ peptides of this disclosure. In some embodiments, the LAG3 and/or GAL3 inhibitory agent is administered to a subject (e.g. a subject with MM) in combination with ex vivo activated T cells and/or APCs specific to the HLA-A2+ or HLA-A24+ peptides of this disclosure. In some embodiments of the above combination therapies, the treatment includes administering both the multipeptide vaccine and the ex vivo activated T cells and/or APCs multiple times to the subject over the course of treatment and in any order. For instance, a patient may be administered a multipeptide vaccine of this disclosure concurrently with the a LAG3 and/or GAL3 inhibitory agent, followed by administration of cells generated by any of the ex vivo immunotherapy approaches of this disclosure, followed by repeated administration of each of the therapy components (multipeptide vaccine, LAG3 and/or GAL3 inhibitory agent, ex vivo activated immune cells) at periodic intervals over the course of treatment (e.g., every few days or weeks).

In some embodiments of any of the above methods, the anti-LAG3 inhibitory agent is an anti-LAG3 antibody, e.g., Relatlimab; and the anti-GAL3 inhibitory agent TD139. In some embodiments of any of the above methods, the anti-LAG3 inhibitory agent is an anti-LAG3 antibody, e.g., Relatlimab; and the anti-GAL3 inhibitory agent is an anti-GAL3 antibody. In some embodiments of any of the above methods, the subject can concurrently be administered an additional therapeutic agent. For instance, the subject may be administered an anti-GAL3 or anti-LAG3 antibody in combination with the multiple vaccine and ex vivo activated immune cells of this disclosure, further in combination with a checkpoint inhibitor (e.g., an anti-PD1 antibody like Pembrolizumab, Nivolumab, or Dostarlimab) and/or an immune agonist (anti-OX40 antibody like PF-04518600, or IBI101 or anti-GITR antibody like BMS-986156), still further in combination with an immunomodulatory drug (e.g., lenalidomide). Moreover, the subject may be concurrently undergoing traditional therapy for a condition of this disclosure (e.g., MM), or may have already received chemotherapy, radiation, corticosteroid treatment and/or continuous checkpoint inhibitor therapy for the condition, or treatment with an agent including, but not limited to, a therapeutic antibody, an immunomodulatory drug, a histone deacetylase (HDAC) inhibitor, an antineoplastic agent, a proteasome inhibitor, an antibody-drug conjugate, a nuclear export inhibitor and a corticosteroid. The therapeutic antibody that can be used as an additional treatment includes, but is not limited to, an anti-PD-L1 antibody (e.g., Durvalumab), an anti-CD38 antibody (e.g., Daratumumab or Isatuximab), an anti-SLAMF7 antibody (e.g., Elotuzumab), an anti-CTLA4 antibody (e.g., Ipilimumab or Tremelimumab), an anti-TIM3 antibody (e.g., Cobolimab), or an anti-VISTA antibody (e.g., SG7 or W0180). The immunomodulatory drug that can be used as an additional treatment includes, but is not limited to pomalidomide, or thalidomide. The HDAC inhibitor that can be used as an additional treatment includes, but is not limited to, citarinostat or panobinostat. The antineoplastic agent that can be used as an additional treatment includes, but is not limited to, cyclophosphamide, etoposide, oxorubicin, liposomal doxorubicin, melphalan, melphalan flufenamide, and bendamustine. The proteasome inhibitor that can be used as an additional treatment includes, but is not limited to, bortezomib, carfilzomib, and ixazomib. The antibody-drug conjugate that can be used as an additional treatment includes, but is not limited to, belantamab mafodotin-blmf. The nuclear export inhibitor that can be used as an additional treatment includes, but is not limited to, selinexor. The corticosteroid that can be used as an additional treatment includes, but is not limited to, dexamethasone or prednisone.

In embodiments where the method increases T lymphocyte responses in a tumor microenvironment while reducing immunosuppression in a human subject, the subject may be administered LAG3 and/or GAL3 inhibitory agents in combination with one or more agents, (e.g., an anti-PD1 antibody, an anti-OX40 antibody, an anti-GITR antibody, etc.). An increase in “T lymphocyte responses” may be an increase in features/phenomenon including but not limited to: T cell proliferation, anti-MM activity of effector T cells, expression of activating costimulatory molecules on T cells etc. The method may also “reduce immunosuppression”, i.e., reduce immune suppressor cells and/or regulatory T cells, block inhibitory molecules on suppressive/regulatory and cancer cells; minimize induction of immune suppressor cells including CD4+ regulatory T cells (Treg); etc.

As defined herein, a “therapeutically effective amount” of an inhibitory agent, peptide, cell, or additional therapeutic agent is an amount of the agent that is capable of producing an immune response in a treated subject, and/or an amount that reduces a clinical feature of the disease (e.g., neoplastic proliferation of plasma cells), and/or an amount that reduces a symptom of a condition of the disclosure. A therapeutically effective amount of an inhibitory agent, peptide, or additional therapeutic agent of this disclosure (i.e., an effective dosage) includes milligram, microgram, nanogram, or picogram amounts of the agent per kilogram of subject or sample weight (e.g., about 1 nanogram per kilogram to about 500 micrograms per kilogram, about 1 microgram per kilogram to about 500 milligrams per kilogram, about 100 micrograms per kilogram to about 5 milligrams per kilogram, or about 1 microgram per kilogram to about 50 micrograms per kilogram).

### EXAMPLES

The practice of the methods and compositions of the disclosure employs, unless otherwise indicated, conventional techniques of molecular biology (including recombinant techniques), cell culture, immunology, cell biology, and biochemistry, which are well within the purview of the skilled artisan. Such techniques are explained in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the methods and compositions of the disclosure. Particularly useful techniques for particular embodiments will be discussed in the sections that follow. The materials, reagents, and methods, further described below, are used in the following examples. The invention, as described in the following examples, do not limit the scope of the invention described in the claims.

### Materials & Methods

### Cell Lines and Preparation of Tumor Cell Lysates or Irradiated Whole Tumor Cells

Multiple myeloma (MM) cell lines U266, McCAR, HSB2, MMIS, RPMI8226, OPM2, H929, ANBL6, OCIMY5, AMO1 and KMS11 were obtained from ATCC (Manassas, VA). The T2 cell line, a human B and T cell hybrid expressing HLA-A2 molecules, was provided by Dr. J. Molldrem (University of Texas M. D. Anderson Cancer Center, Houston, TX). The cell lines were cultured in DMEM media supplemented with 10% fetal calf serum (FCS; BioWhittaker, Walkersville, MD), 100 IU/ml penicillin, and 100 μg/ml streptomycin (Gibco-Life Technologies, Rockville, MD). A mixture of ten MM cell lines was utilized to prepare tumor cell lysates by repeated (10×) cycles of freeze (−140° C.)/thaw (37° C.) or prepared as irradiated (20 Gy) whole tumor cells as sources of MM antigen stimulation.

### Reagents

Fluorochrome conjugated anti-human monoclonal antibodies (mAb) specific to CD3, CD4, CD8, CD11b, CD11b, CD14, CD15, CD25, CD28, CD33, CD38, CD69, CD138, FOXP3, HLA-DR, PD1, PD-L1, PD-L2, CTLA4, LAG3, TIM3, VISTA, ICOS, OX40, GITR, GAL-3, GAL-9, ICOS-L, HLA-DP/DQ/DR, CCR7, CD45RO, CD69, CD107a or IFN-γ were purchased from Becton Dickinson (BD) (San Diego, CA), LifeSpan Bioscience (Seattle, WA), BioLegend (San Diego, CA) or eBioscience (San Diego, CA). Live/Dead Aqua fixable cell stain kit was purchased from Molecular Probes (Grand Island, NY). Recombinant human GM-CSF was obtained from Immunex (Seattle, WA), and human IL-2, IL-4, IFN-α and TNF-α were purchased from R&D Systems (Minneapolis, MN). Clinical grade checkpoint inhibitors (anti-PD1 antibody Nivolumab and anti-LAG3 antibody Relatlimab) or immune agonists (anti-OX40, anti-GITR) were provided by Bristol-Myers Squibb (New York, NY). Anti-GAL3 antibody was purchased from BD (Clone B2C10; Cat Nos.: 565676-565678, and 565682)

Bone Marrow (BM) or Peripheral Blood (PB) Samples from MGUS, SMM or MM Patients or Healthy Donors

BM aspirates and peripheral blood (PB) samples were obtained from patients with MGUS [BM: N=5, PB: N=5] and SMM [BM: N=5, PB: N=5] and patients with MM (Newly Diagnosed [BM: N=15, PB: N=10], Relapsed [BM: N=12, PB: N=12], Relapsed/Refractory [BM: N=15, PB: N=12]) after informed consent, in accordance with the Declaration of Helsinki, and with approval by the Institutional Review Board at Dana-Farber Cancer Institute (Boston, MA). In addition, healthy individuals BM [N=5] or leukapheresis [N=5] products were purchased from either AllCells (Alameda, CA) or the Blood Donor Center at Boston Children's Hospital (Boston, MA), respectively. Mononuclear cells were isolated from BM (BMMC) or PB (PBMC) by standard density gradient centrifugation using Ficoll-Paque™ Plus (Amersham Pharmacia Biotech AB, Uppsala Sweden) and used in these studies.

### Phenotypic Characterization of Immune and Regulatory Cell Subsets and Expression of Checkpoints or Costimulatory Molecules

BMMC or PBMC from patients with MGUS, SMM, MM or healthy individuals were evaluated by flow cytometry analyses by staining cells with fluorochrome-conjugated mAb specific to each cell surface antigen for 30 minutes at room temperature, followed by LIVE/DEAD reagent staining to confirm viability. Regulatory T cells (Treg) were identified by cell surface staining (CD3, CD4, CD8, CD25), permeabilized using Foxp3/Transcription Factor Staining Buffer Set (eBioscience) and stained for intracellular FOXP3 expression. Myeloid-derived-suppressor cells (MDSC) were identified as G-type MDSC (CD11b+CD33+ HLA-DRlow/−CD14−CD15+) and M-type MDSC (CD11b+CD33+ HLA-DRlow/−CD14+CD15−). Cells were acquired using a BD Fortessa X-20 (BD Biosciences) flow cytometer, and the data was analyzed using DIVA™ v8.0 (BD) or FlowJo v10.0.7 (Tree star, Ashland, OR) software.

Generation of MM-Specific CTL Ex Vivo with Immunogenic XBP1 CD138 CSI Peptides

XBP1/CD138/CS1-specific CTL were generated ex vivo after four cycles of weekly stimulation of HLA-A2⁺ CD3⁺ T lymphocytes (N=5) with a cocktail of four peptides containing heteroclitic XBP1 US₁₈₄₋₁₉₂ (YISPWILAV (SEQ ID NO: 24)), heteroclitic XBP1 SP₃₆₇₋₃₇₅ (YLFPQLISV (SEQ ID NO: 25)), native CD138₂₆₀₋₂₆₈ (GLVGLIFAV (SEQ ID NO: 26)) and native CS1₂₃₉₋₂₄₇ (SLFVLGLFL (SEQ ID NO: 27)), as described previously.

### Cell Proliferation by Carboxy Fluorescein Succinimidyl Ester (CFSE) Tracking

Proliferation of specific cell populations was evaluated using CFSE-based proliferation assays. In brief, MM patient BMMC, PBMC or XBP1/CD138/CS1-specific CTL were labeled with CFSE (Molecular Probes, Eugene, OR) and incubated with clinical grade checkpoint inhibitor (1 μg/ml) or immune agonist (1 μg/ml) in the presence of low dose (20 units) IL-2, with or without stimulation with irradiated MM cells (patients' cells, cell line) or MM lysates. After 4-7 days incubation, the cells were stained with LIVE/DEAD reagent and specific fluorochrome conjugated mAbs, washed, fixed in 2% paraformaldehyde, and acquired using a LSRII Fortessa™ flow cytometer.

### XBP1 CD138 CSI-Specific CTL Functional Activities Measured by CD107a Degranulation and Intracellular IFN-γ Production

The anti-tumor activities of MM-specific CTL were measured by CD107a degranulation and IFN-γ production against MM. In brief, XBP1/CD138/CS1-specific CTL were treated with clinical grade anti-PD1 or anti-LAG3 (1 μg/ml) for 24 hours. The cells were then cultured with U266 MM cells in the presence of CD107a mAb. After 1-hour incubation, Brefeldin A (BD) and Monensin (BD) were added, and cultures were incubated for an additional 5 hours. Cells were harvested, washed in PBS, stained with LIVE/DEAD reagent, washed, incubated with fluorochrome conjugated mAb to identify T cells, allowing for assays of their functional activity against MM. After surface staining, cells were fixed/permeabilized, stained for Th1 cytokines, washed with Perm/Wash solution (BD), and analyzed by flow cytometry. MP-CTL specific CD107a degranulation and Th1 cytokine production were analyzed using DIVA™ v8.0 or FlowJo v10.0.7 software.

### Statistical Analysis

Results are presented as mean±SE. Groups were compared using an unpaired Student's t-test. Differences were considered significant when p<0.05.

### Example 1. Impact of Immune Modulator Treatment on Proliferation of MM Patient T Cells Expressing Checkpoint or Costimulatory Molecules

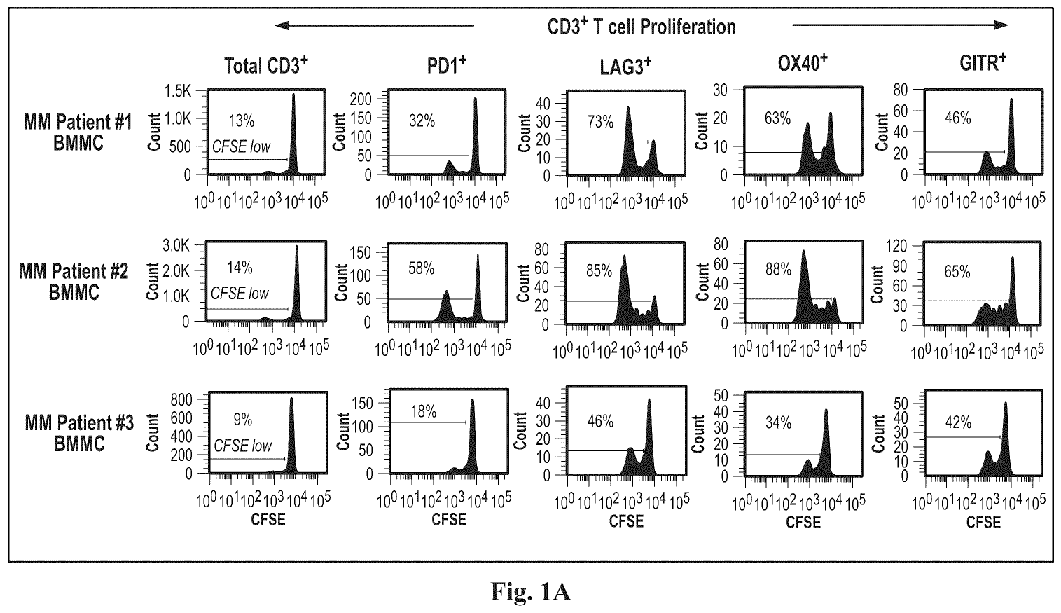

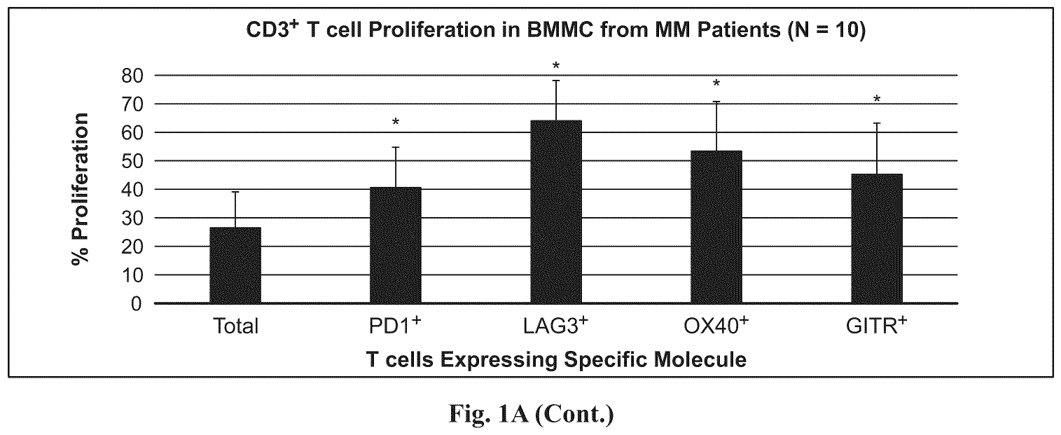

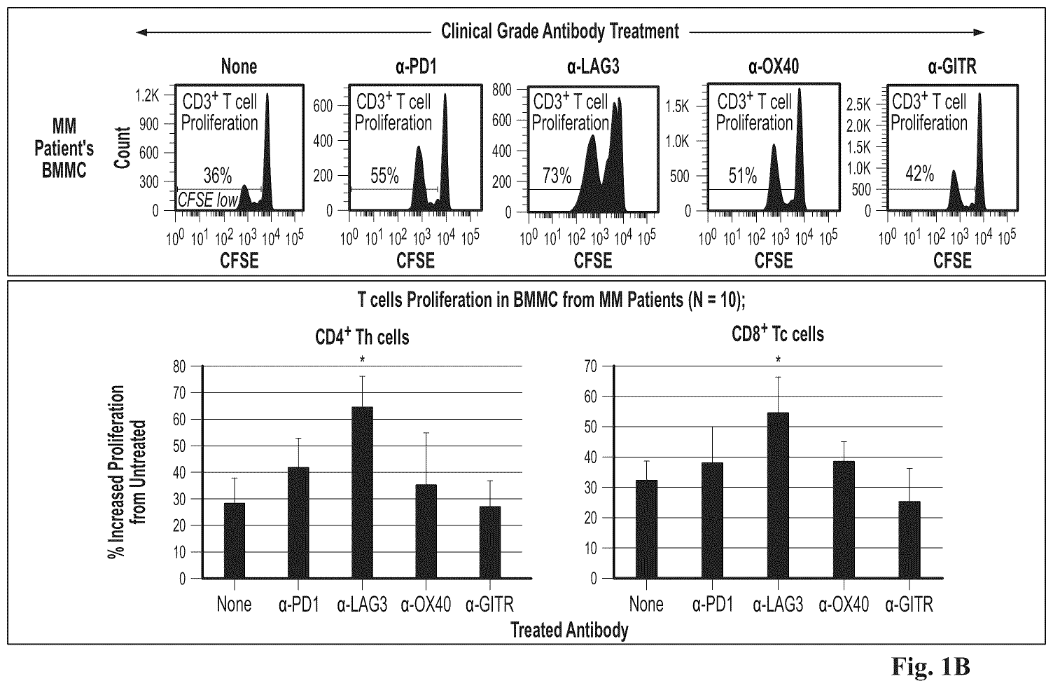

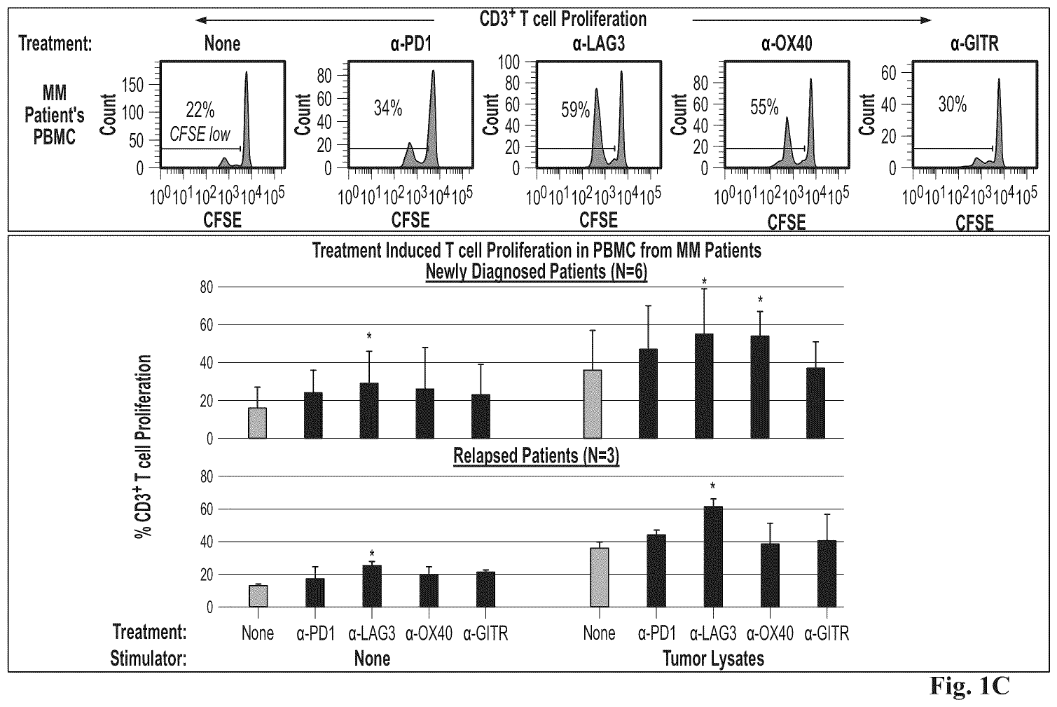

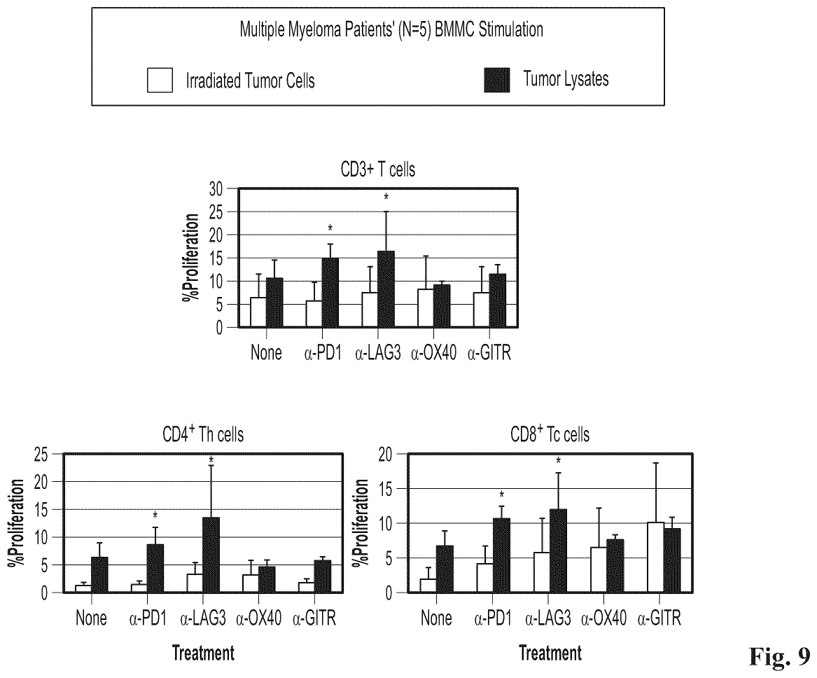

Proliferation in response to low dose IL-2 in specific T cell subsets in bone marrow mononuclear cells (BMMC) or Peripheral Blood Mononuclear Cells (PBMC) from patients with newly diagnosed, relapsed or relapsed/refractory MM was evaluated using Carboxy Fluorescein Succinimidyl Ester (CFSE)-based assays. Proliferation of CD3⁺ T cells expressing PD1, LAG3, OX40 or GITR was significantly (*p<0.05) higher as compared to total CD3⁺ T cells in MM patient BMMC. In BMMC from patients (N=10) with newly diagnosed, relapsed or relapsed/refractory MM, T cell subsets expressing the LAG3 immune checkpoint demonstrated the highest (*p<0.05) proliferation (FIG. 1A; Histograms, Bar graph). Next, the impact of clinical grade immune modulators on T cell proliferation within BMMC from patients with newly diagnosed, relapsed or relapsed/refractory MM (N=10) was assessed. Overall, CD3⁺ T cell proliferation was triggered by treatment with each clinical grade antibody (PD-1, LAG3, OX40, GITR) compared to untreated control (FIG. 1B). Of note, a significant increase (*p<0.05) in proliferation of CD4+ Th cells was induced by treatment with anti-PD1 or anti-LAG3; and increased (*p<0.05) proliferation of CD8+ Tc cells after treatment with anti-LAG3 or anti-OX40. The highest proliferation in both CD4+ Th cells and CD8+ Tc cells was induced by anti-LAG3 treatment (FIG. 1B; Histograms, Bar graphs). Next, MM patient T cell proliferation in response to tumor lysates from ten different MM cell lines, in the presence or absence of immune modulator, was examined. As shown in FIG. 1C, PBMC from patients with newly diagnosed (N=6) or relapsed (N=3) MM treated with anti-LAG3 had significantly (*p<0.05) higher T cell proliferation than with the other clinical grade immune modulators anti-PD1, anti-OX40, anti-GITR, either in the presence or absence of MM lysate stimulation. In addition, BMMC from MM patients (N=5) treated with anti-LAG3 had significantly higher (*p<0.05) T cell proliferation than with the other clinical grade immune modulators, upon stimulation with the mixture of ten different MM cell lines, either as irradiated whole cells or tumor lysates (FIG. 9). The tumor lysates induced a greater T cell response in MM patients' BMMC (N=5) than irradiated whole tumor cells, which was enhanced to a greater extent by checkpoint inhibitors (anti-LAG3>anti-PD1) than by immune agonists (anti-OX40, anti-GITR) (FIG. 9).

Taken together, these data indicate the therapeutic potential of LAG3 blockade to effectively augment T cell proliferation directed against MM.

### Example 2. Decreased Effector CD4+ Th Cells, Increased Regulatory and Immune Suppressor Cells, and Upregulation of Immune Checkpoints in Active MM Patients Compared to MGUS/SMM Patients or Healthy Donors

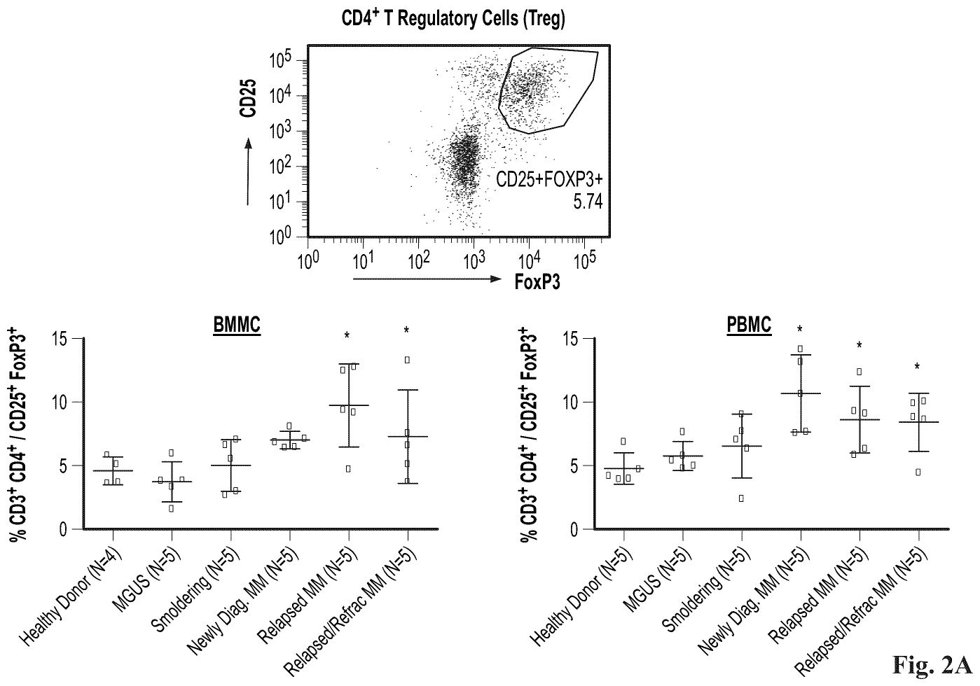

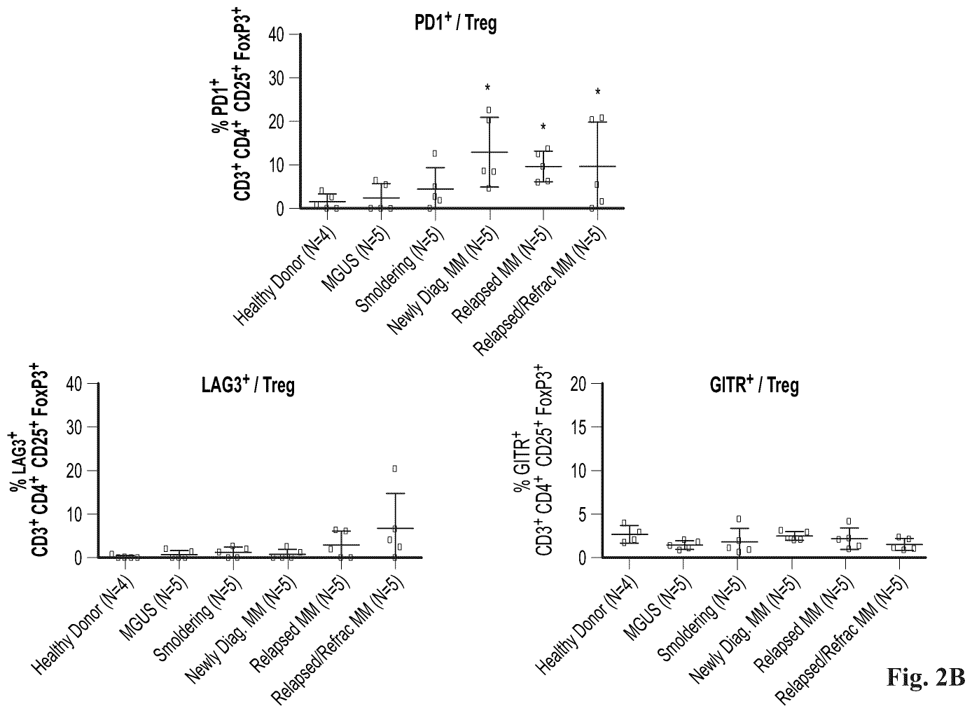

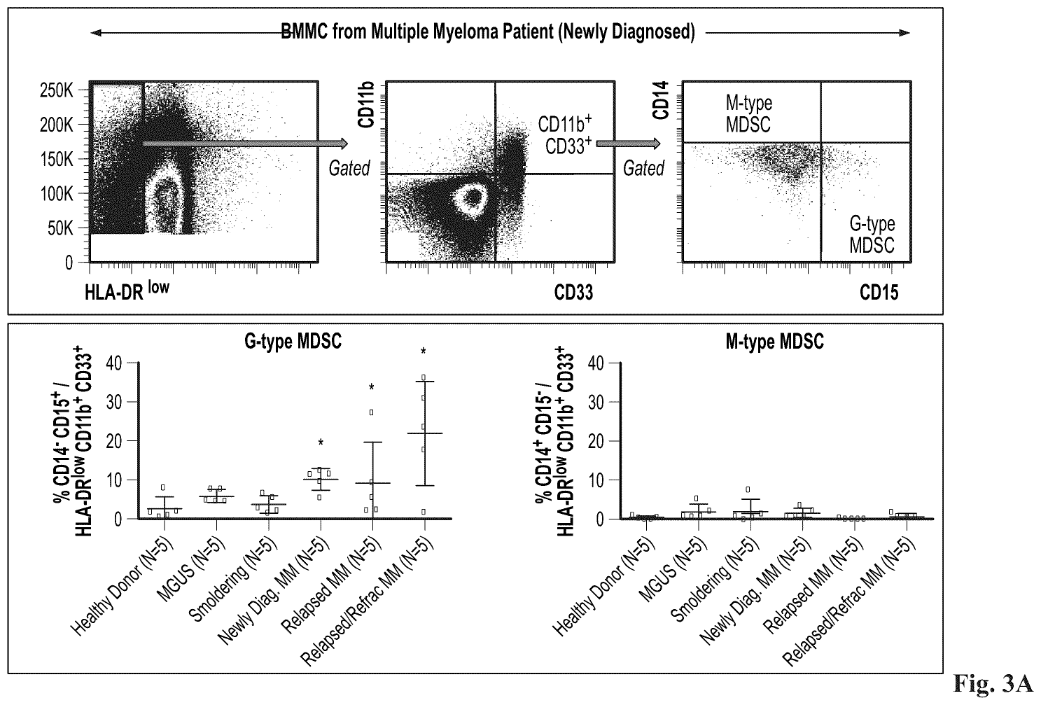

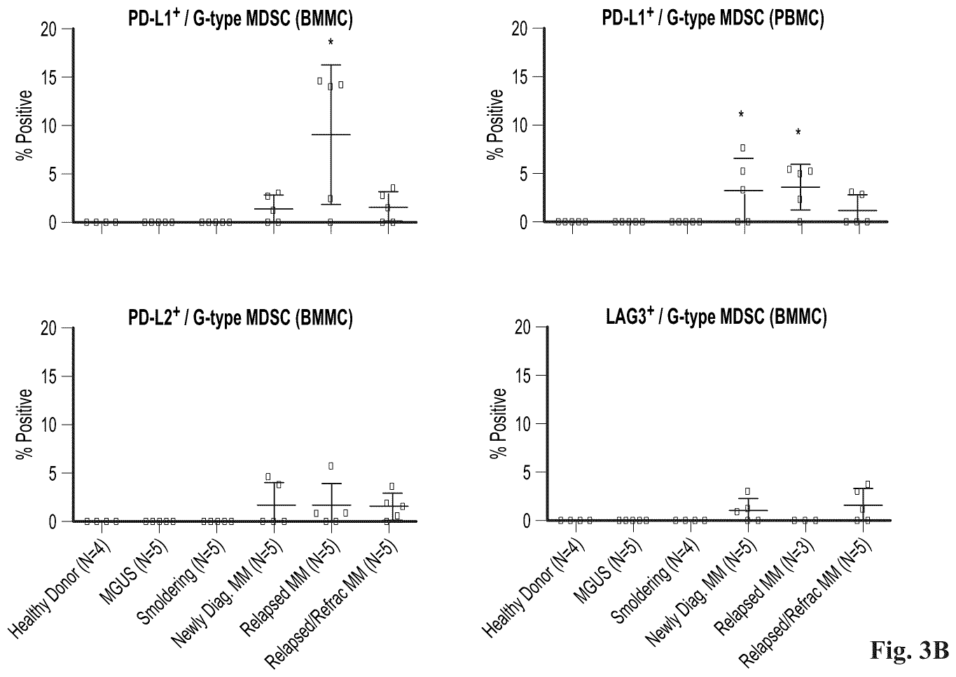

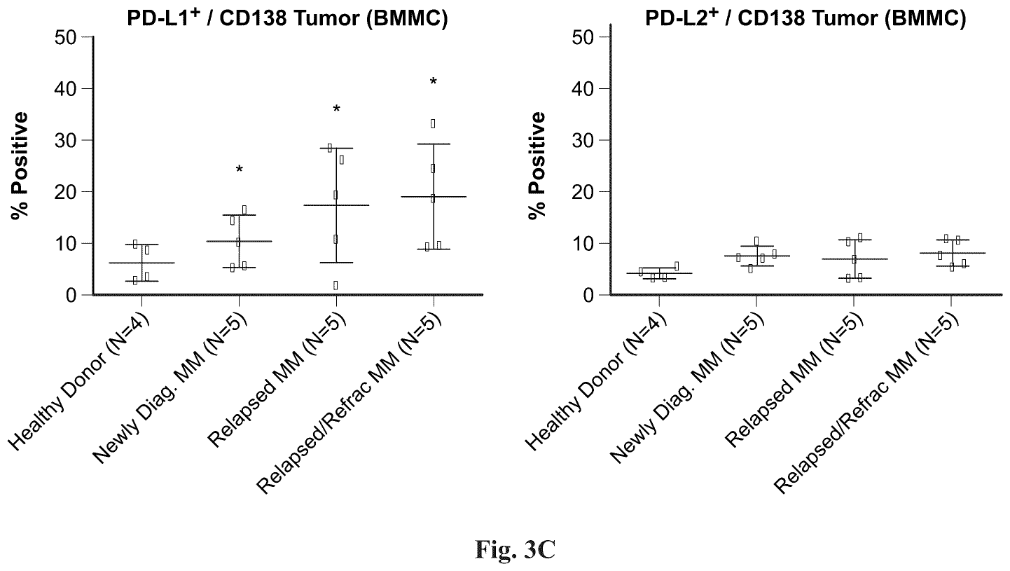

Immune effector, regulatory/suppressor, and tumor cells were evaluated for expression of key immune checkpoints using freshly isolated BMMC and PBMC from patients with MGUS, SMM or MM, as well as normal healthy individuals. Compared to MGUS/SMM patients or healthy individuals, MM patients' (newly diagnosed, relapsed, relapsed/refractory) BMMC and PBMC had significantly (*p<0.05) decreased CD4⁺ Th, but not CD8+ Tc cells (data not shown); as well as increased CD4⁺ Treg (CD3⁺CD4⁺/FOXP3⁺CD25⁺) (FIG. 2A). Of note, PD-1 was more highly expressed on CD4⁺ Treg within BMMC from MM patients than patients with MGUS/SMM or healthy individuals and demonstrated higher expression of PD-1 than LAG3 or GITR (FIG. 2B). Next, G-type Myeloid-derived suppressor cells (MDSC) (CD11b⁺CD33⁺HLA-DR^{low/-} CD14⁻ CD15⁺), but not M-type MDSC (CD11b⁺ CD33⁺ HLA-DR^{low/-}CD14⁺ CD15⁻), were shown to be significantly (*p<0.05) increased in BMMC of MM patients (highest in relapsed/refractory MM) compared to MGUS/SMM patients or healthy donors (FIG. 3A). Moreover, G-type MDSC in BMMC and PBMC of MM patients expressed significantly higher levels of PD-L1 than PD-L2 or LAG3 (FIG. 3B). Finally, CD138⁺ MM cells (newly diagnosed, relapsed, relapsed/refractory) were shown to have significantly (*p<0.05) higher expression of PD-L1, but not PD-L2, than healthy donors (FIG. 3C). In addition, CD4⁺ Th cells and CD8⁺ Tc cells in MM patients had significantly higher PD1 expression compared to MGUS/SMM patients or healthy individuals (data not shown).

Taken together, these data indicate the heterogeneity in the proportion of immune cell subsets among patients with MGUS, SMM or MM, and healthy individuals: decreased effector cells, increased Treg and G-type MDSC, as well as upregulation of immune checkpoints on effector, regulatory, and CD138⁺ MM cells, are observed in MM patients compared to patients with MGUS or SMM or healthy individuals.

### Example 3. Higher Intracellular than Surface Expression of Immune Checkpoints in MM Patient Bone Marrow

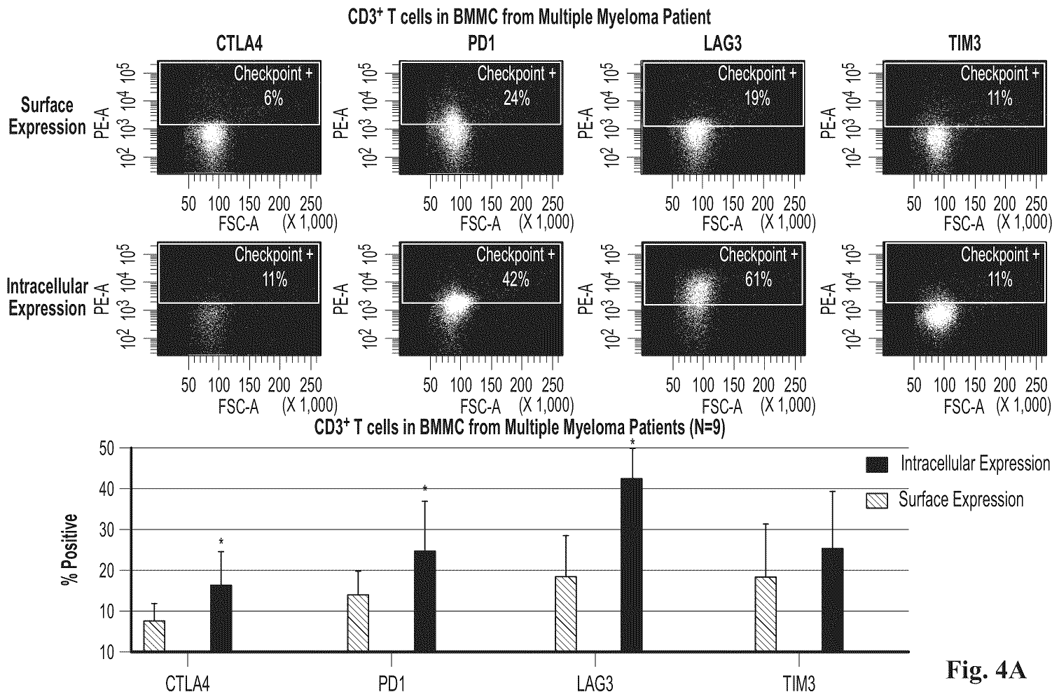

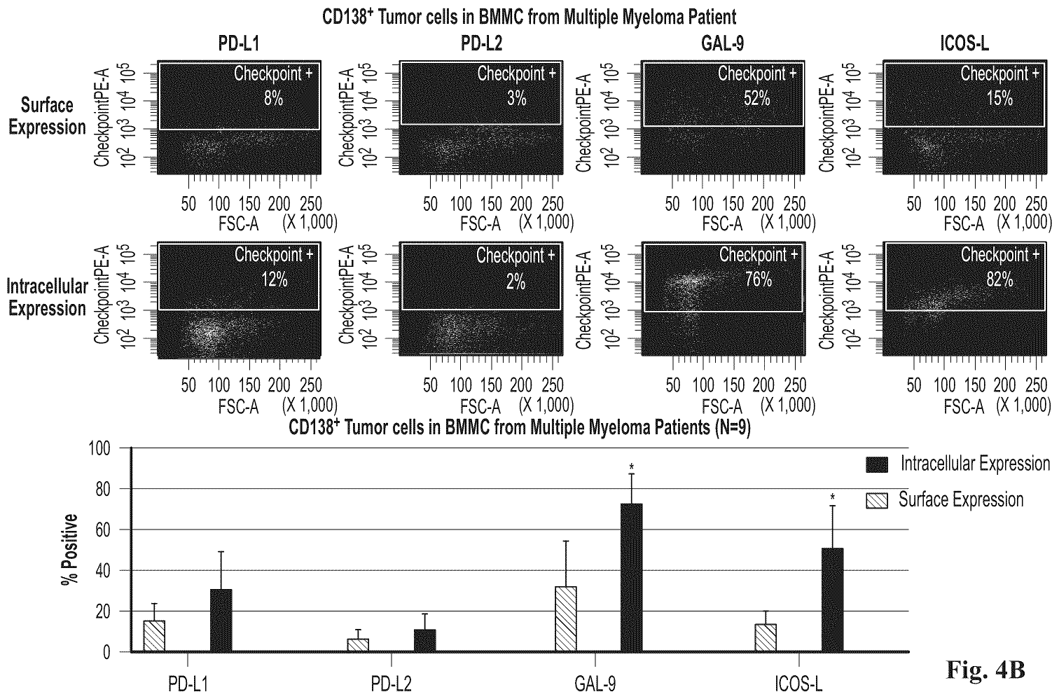

To better understand potential mechanisms of resistance to checkpoint inhibitor therapy in MM, the distribution and localization (cell surface vs. intracellular) of key immune checkpoints in BMMC from MM patients (N=9) were examined. CD3⁺ T cells were found to express PD1 and LAG3 more highly than CTLA4 and TIM3, with significantly (*p<0.05) greater intracellular CTLA4, PD1 and LAG3 expression than cell surface levels (FIG. 4A; Histograms, Bar graph). In contrast, surface and intracellular expression levels of TIM3 were found to be similar. On the CD138 MM cells, GAL-9 and ICOS-L were found to be expressed more highly than PD-L1 and PD-L2; with higher intracellular than cell surface expression of PD-L1, PD-L2, GAL-9 and ICOS-L (FIG. 4B; Histograms, Bar graph).

Taken together, these data provide support for an extended treatment protocol with checkpoint inhibitors to overcome the high intracellular reservoir of immune checkpoints, and thereby overcome immunosuppression and improve outcomes in MM.

### Example 4. Induction of Another Checkpoint Expression and Treg Triggered by Treatment with Checkpoint Inhibitor or Immune Agonist

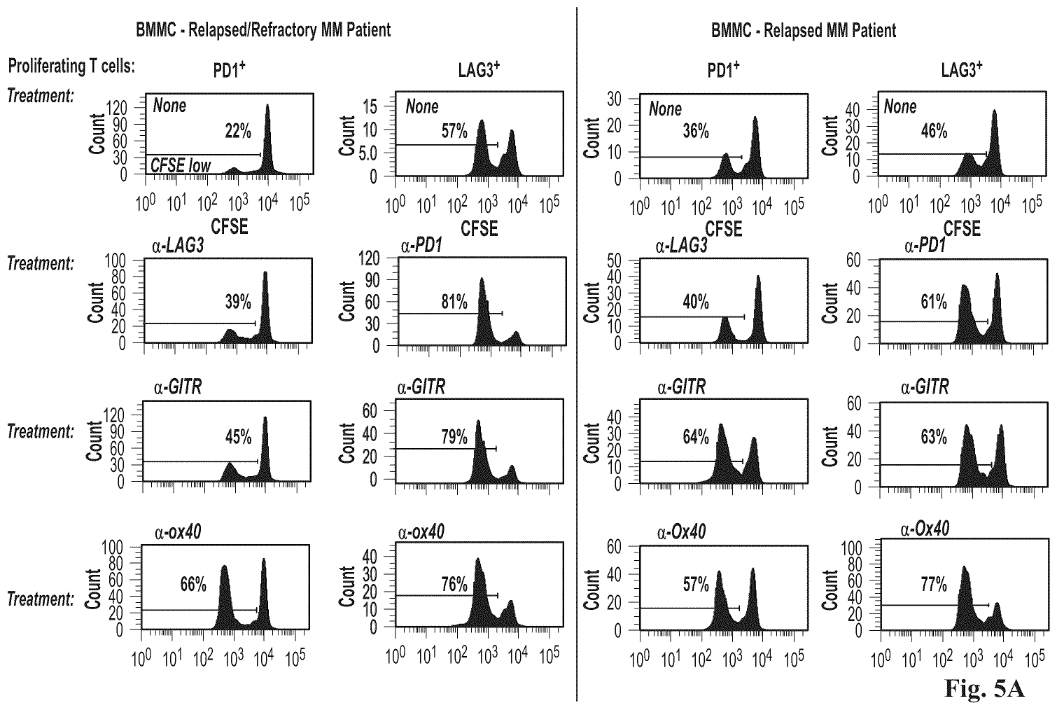

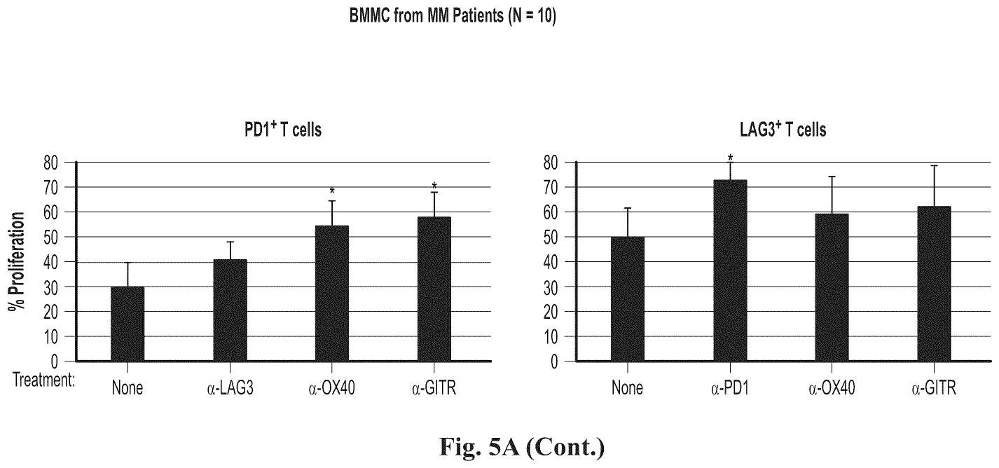

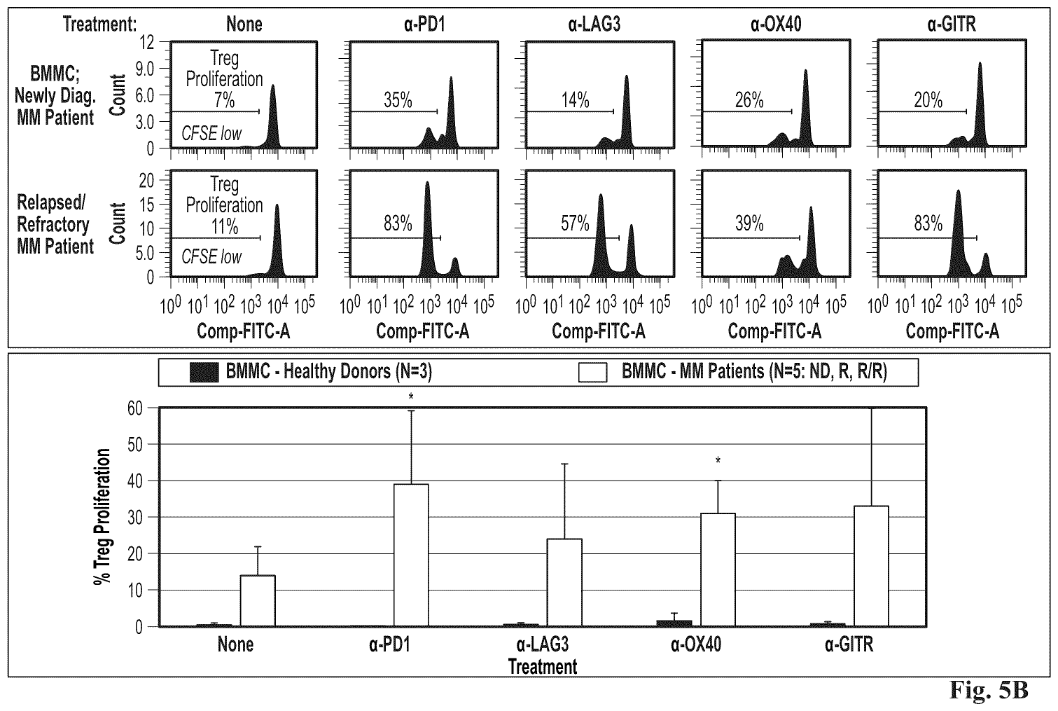

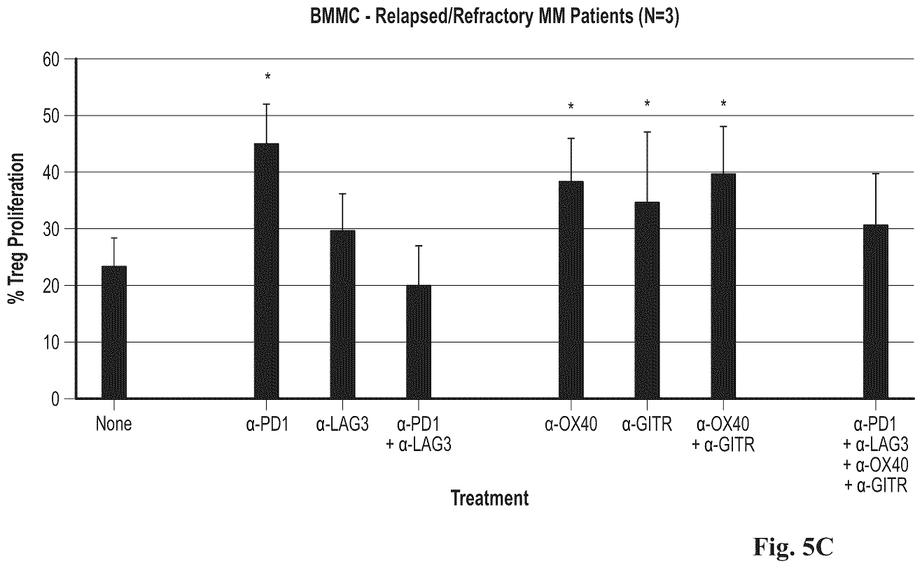

To further elucidate potential mechanisms of resistance to checkpoint inhibitor or immune agonist therapy in MM patients, the effects of clinical grade modulators on effector and regulatory T cell subsets in tumor microenvironment were evaluated. Importantly, treatment of MM patients' (N=10) BMMCs with the specific mAb targeting PD1, LAG3, OX40 or GITR were found to induce upregulation of PD1 and LAG3 expression on T cells (FIG. 5A; Histograms, Bar graph). Of note, anti-PD1 triggered proliferation of T cells expressing an alternative immune checkpoint to a greater extent than anti-LAG3 treatment. Moreover, checkpoint inhibitor or immune agonist treatment of MM patients' (N=5) BMMCs were found to increase Treg proliferation, with a significantly (*p<0.05) high induction by anti-PD1 or anti-OX40 and the lowest induction by anti-LAG3 (FIG. 5B; Histograms, Bar graph).

The impact of single agent or combination modulator treatment on Treg expansion in the MM microenvironment were also investigated (FIG. 5C). Treatment of MM patients' (N=3; relapsed/refractory) BMMC with single agent anti-PD1, anti-OX40 or anti-GITR triggered a significant (*p<0.05) expansion of Treg cells. Treg cell proliferation was found to be decreased upon combination treatment with checkpoint inhibitors, which was not detected in combination treatment with immune agonists. Further, among various combination treatments evaluated, the lowest level of Treg proliferation was noted with anti-PD1 plus anti-LAG3.

Taken together, these results indicate a potential mechanism of immune resistance to checkpoint therapy whereby treatment with a checkpoint inhibitor induces immune suppressive cells and expression of an alternative checkpoint in MM patients BMMC, suggesting the need for combination modulator treatment to overcome resistance to single agent immunotherapy approaches.

### Example 5. Increased Functional Anti-MM Activity of XBP1/CD138/CS1-Specific CTL Treated with Anti-LAG3

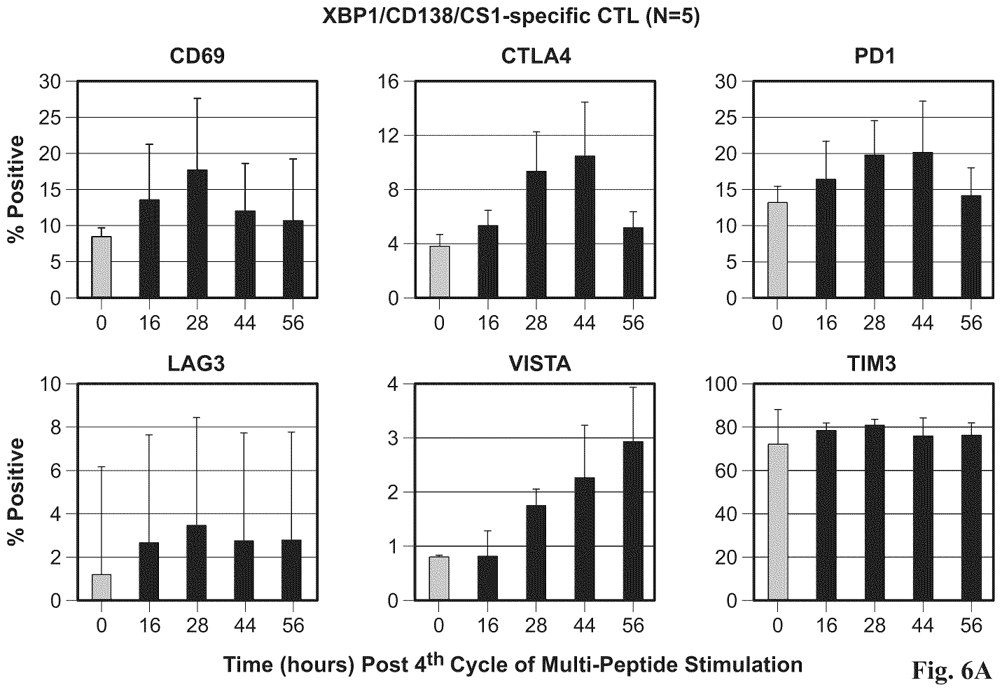

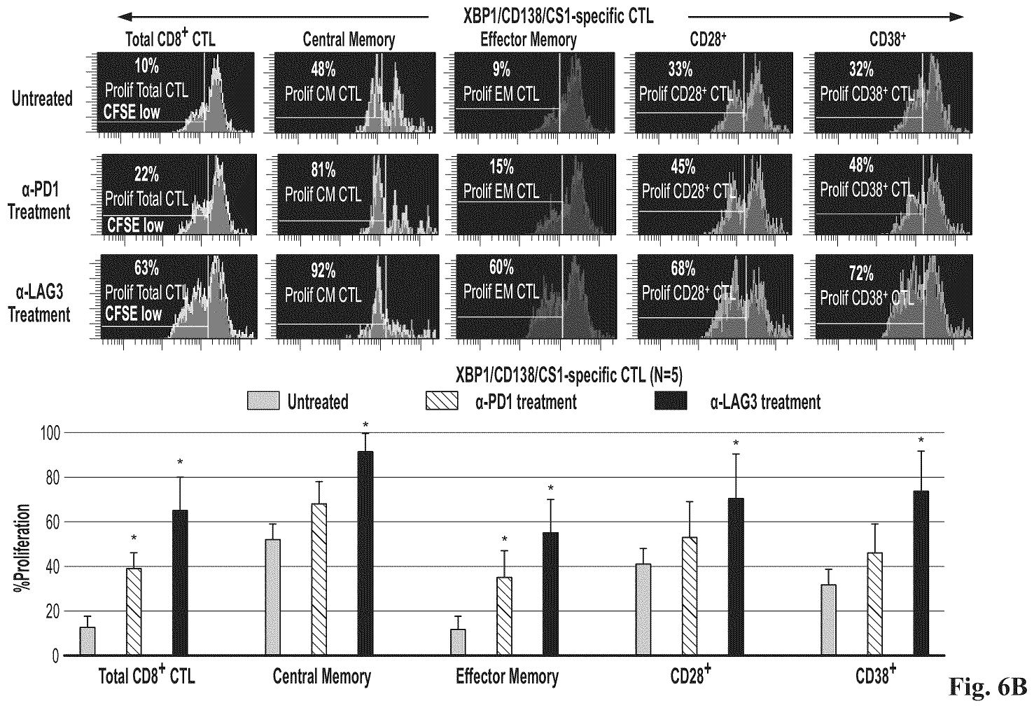

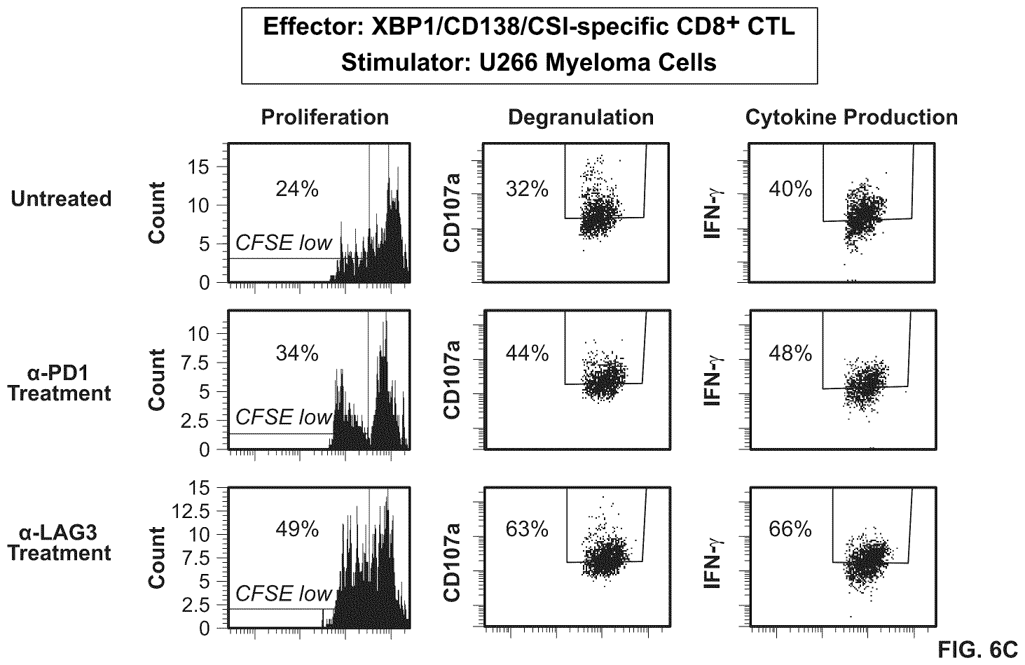

The functional significance of immune modulator therapy was evaluated by examining its impact on anti-tumor activity of MM-specific CTL generated with HLA-A2 XBP1/CD138/CS1 peptides including heteroclitic XBP1 US₁₈₄₋₁₉₂ (YISPWILAV; SEQ ID NO: 24), heteroclitic XBP1 SP₃₆₇₋₃₇₅ (YLFPQLISV; SEQ ID NO: 25), native CD138₂₆₀₋₂₆₈ (GL VGLIFAV; SEQ ID NO: 26), and native CS1₂₃₉₋₂₄₇ (SLFVLGLFL; SEQ ID NO: 27), as described previously. See, e.g., Bae J, et al.,. Leukemia 2011(1); 25: 1610-1619; Bae J, et al. Br J Haematol 2011(2); 155: 349-361; Bae J, et al. Br J Haematol 2012; 157: 687-701; Bae J, et al. Clin Cancer Res 2012; 18: 4850-4860. Phenotypic analyses after four cycles of weekly stimulation with peptides was found to demonstrate time-dependent increased expression of CD69 activation marker and CTLA4, PD1, LAG3 and VISTA immune checkpoints on XBP1/CD138/CS1-specific CTL (N=5) (FIG. 6A). In response to stimulation with HLA-A2 matched MM cells (U266), the central memory CD8⁺ T cell subset was found to display the highest proliferation (48%). Importantly, the XBP1/CD138/CS1-CTL treated with clinical grade anti-LAG3 or anti-PD1 antibodies was found to significantly increase proliferation (α-LAG3>α-PD-1) (*p<0.05) of total CD8⁺ T cells as well as Central Memory (CM), Effector Memory (EM), CD28⁺ and CD38⁺ CTL subsets (FIG. 6B; Histograms, Bar graph [N=5]). The treatment with each checkpoint inhibitor was also found to increase anti-tumor activities of XBP1/CD138/CS1-CTL against MM cells, evidenced by increased CD107a degranulation and IFN-γ production, with the highest anti-tumor activities induced by anti-LAG3 treatment (FIG. 6C).

Taken together, these results further support the ability of LAG3 blockade to augment anti-MM immune responses including antigen-specific memory CTL, their cytotoxic activities, and Th1 cytokine production against tumors.

### Example 6. Surface and Intracellular Expression of LAG3 Ligands, GAL-3 and HLA-DP/DQ/DR, in CD138+ Cells in MM Patient BMMC, and Induction of Specific CD8+ Tc Proliferation by Blocking GAL-3

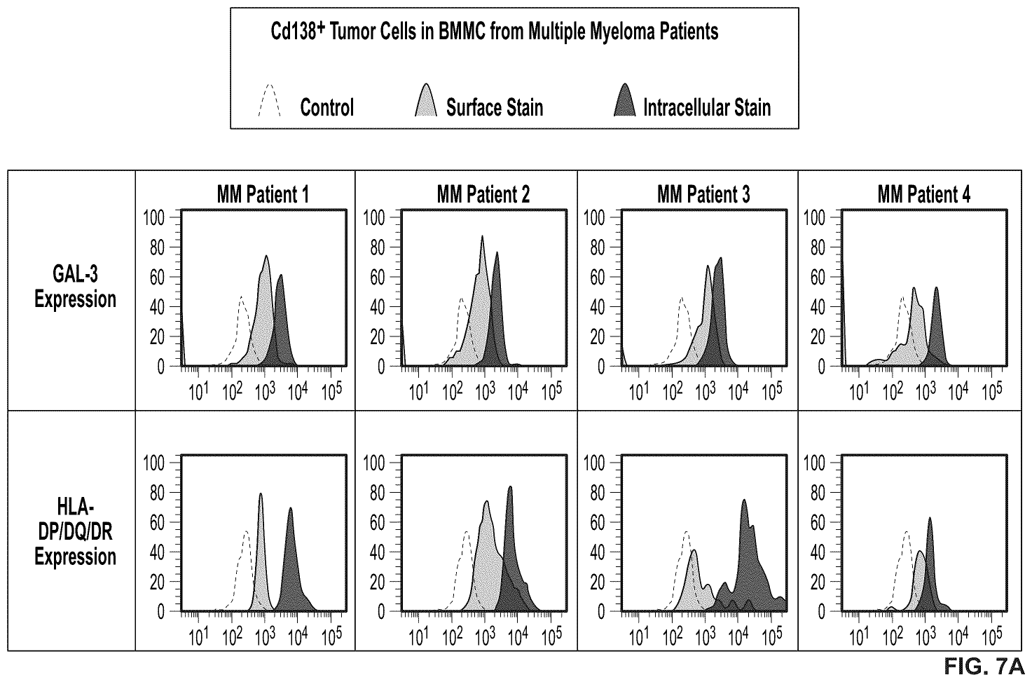

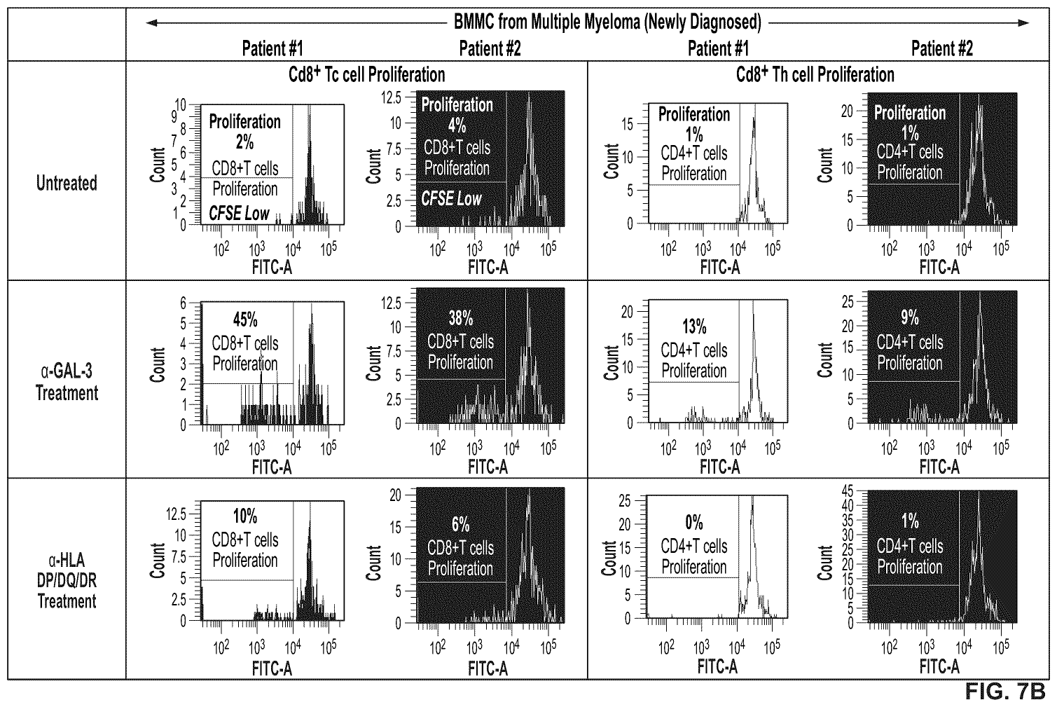

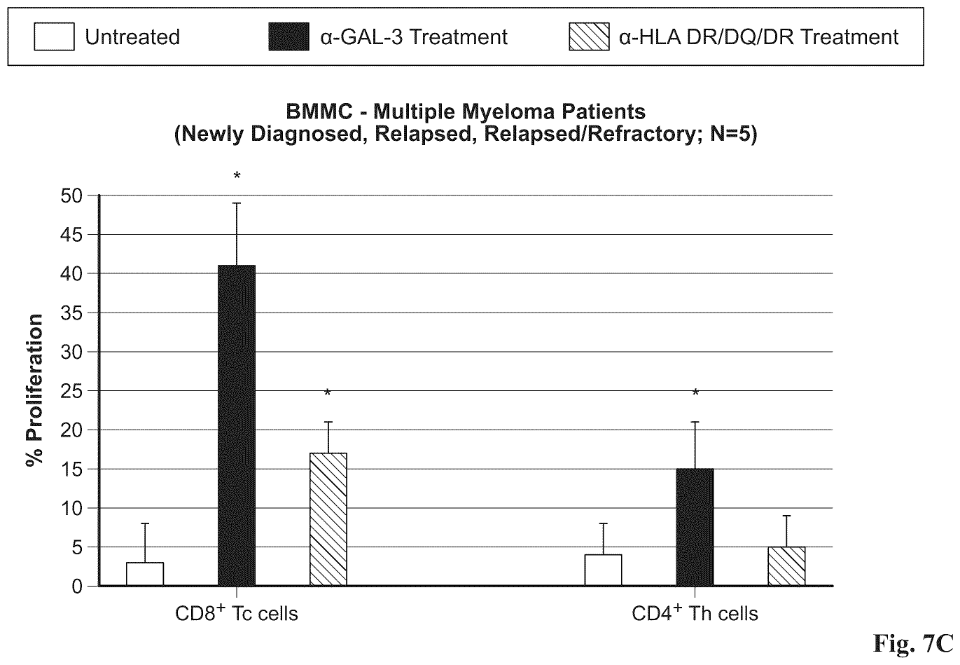

Having shown the functional significance of the LAG3 immune checkpoint in MM, LAG3 ligands GAL-3 and HLA-DP/DQ/DR were next assessed. The expression and role of each of the LAG3 ligands was analyzed in BMMC from MM patients (N=4; newly diagnosed, relapsed, relapsed refractory). CD138+MM cells were found to express both GAL-3 and HLA-DP/DQ/DR, with greater expression seen by higher median fluorescence intensity (MFI) intracellularly than on the cell surface (FIG. 7A). Next, the functional significance of GAL-3 and HLA-DP/DQ/DR blockade in MM was examined. Treatment of newly diagnosed MM patient BMMC with anti-GAL-3 antibodies, but not with anti-HLA-DP/DQ/DR antibodies, was found to induce proliferation of T cells, including CD8+Tc cells (45%: Patient #1, 38%: Patient #2) and CD4+Th cells (13%: Patient #1, 9%: Patient #2) (FIG. 7B). Additional analyses of BMMC from MM patients (newly diagnosed, relapsed, relapsed/refractory; N=5) showed a significant (*p<0.05) increase in proliferation of CD8+Tc and CD4+Th cells (CD8+>CD4+) triggered by GAL-3 blockade (FIG. 7C).

Taken together, these results indicate the potential benefit of GAL-3 blockade in MM patients to induce T cells-specific responses with a high level of CD8+ T cells proliferation.

### Example 7. Induction of MM-Specific CD8+ T Cell Proliferation and Anti-Tumor Activities by Blocking GAL-3 on MM Cells

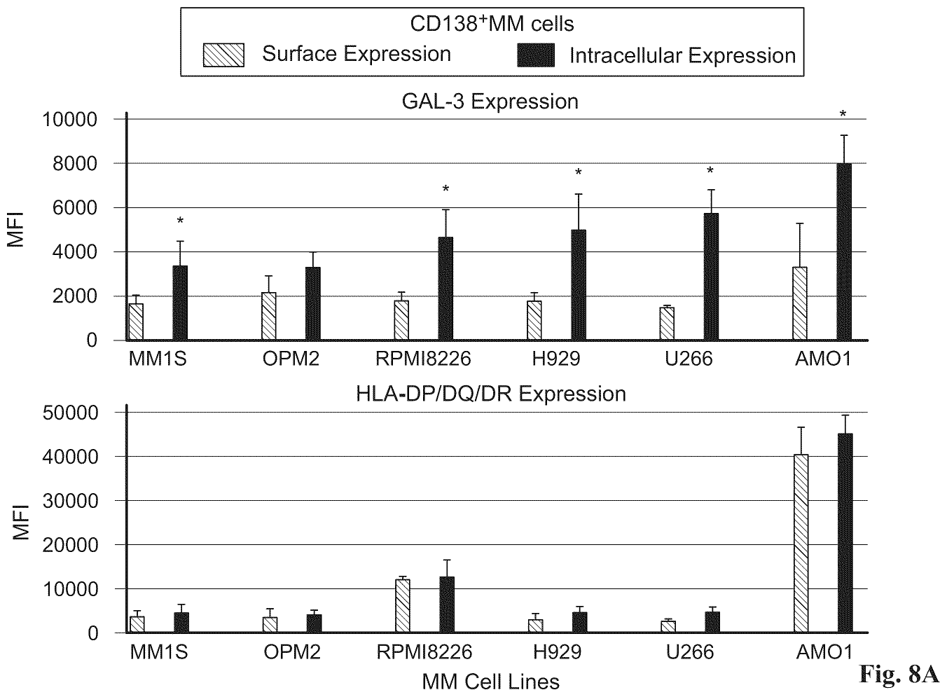

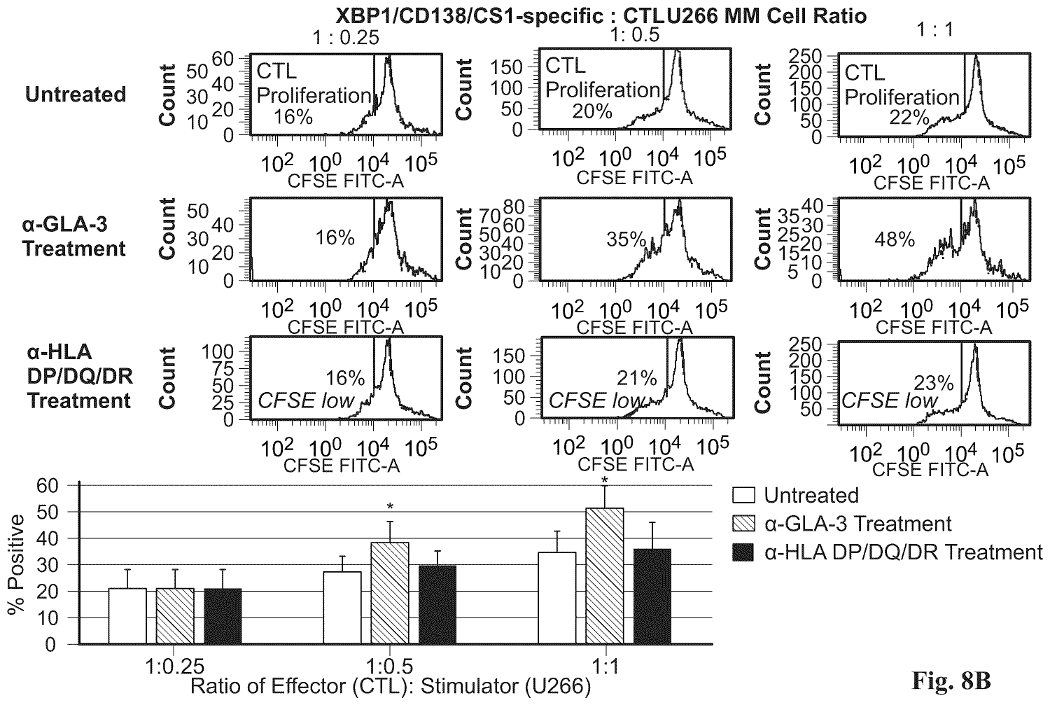

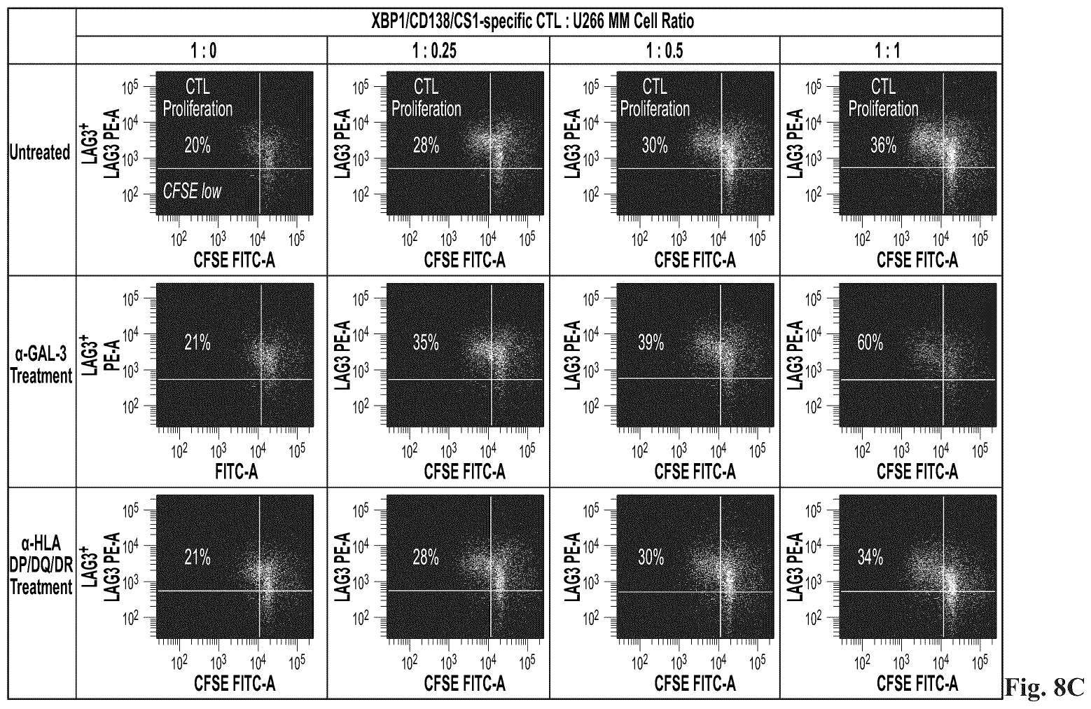

Finally, the impact of inhibiting LAG3-ligand on MM-specific CTL activities was examined. First, GAL-3 and HLA-DP/DQ/DR expression was evaluated in MM cell lines (MM1S, OPM2, RPMI8226, H929, U266, AMO1). Overall, a higher HLA-DP/DQ/DR surface and intracellular expression was detected than GAL-3 expression, whereas GAL-3 displayed a greater (*p<0.05) level of intracellular than cell surface expression (N=3) (FIG. 8A). Next, the functional impact of GAL-3 or HLA-DP/DQ/DR blockade was examined on the specific proliferation and anti-tumor activities of MM-specific CD8+ CTL against MM cells in an HLA-A2-specific manner. As shown in FIG. 8B, proliferation of HLA-A2 XBP1/CD138/CS1-specific CTL was found to be enhanced in response to HLA-A2+U266 MM cells upon the treatment with anti-GAL-3 antibodies, but not with anti-HLA-DP/DQ/DR antibodies, in an effector (CTL):target (MM cells)-dependent manner (1:1>1:0.5>1:0.25) (Histograms, Bar graphs [N=5]). Importantly, the specific blockade of GAL-3 in MM cells was found to further increase proliferation of LAG3 expressing XBP1/CD138/CS1-CTL (FIG. 8C), suggesting an alternative escape mechanism after anti-GAL-3 therapy in MM patients.

Taken together, these results identify the functional relevance of blocking GAL-3 on MM cells as a means to enhance effector T cell activities, and also provide the rationale for targeting GAL-3 (on MM tumor cells) in combination with LAG3 (on effector T cells) to further enhance MM-specific immune responses and anti-tumor activities.

### Discussion of Above Examples

Understanding the biologic and immune sequelae of tumor cell interaction with accessory and immune cells in the tumor microenvironment is crucial for the development of successful cancer immunotherapies. Effective therapeutic strategies in MM may not only target tumor and tumor-promoting accessory cells, but also abrogate mechanisms mediating immunosuppression in the bone marrow milieu. The role of accessory cells (MDSC, plasmacytoid dendritic cells, Treg, osteoclasts) in promoting tumor cell growth, survival and drug resistance, as well as conferring immunosuppression in MM has been delineated. The results in the Examples above characterize the distribution, location, and expression levels of immune checkpoints not only on effector T cells, but also on MM cells and immune regulatory/suppressor cells in the BM and PB of patients with MM (newly diagnosed, relapsed, relapsed/refractory), premalignant diseases (MGUS, SMM), and healthy individuals. These analyses reveal key differences in the frequency of cellular subsets (immune effector, regulatory/suppressor vs. tumor cells) and expression of immune checkpoints/agonists in patients with active MM compared to MGUS/SMM and healthy donors.

Since effective immunotherapy depends upon robust effector T cell function, the presence and function of endogenous T cell subsets in MM patient BM and PB were first defined. Although proliferating CD3⁺ T cells in the presence of IL-2 or MM cell lysates were found to express multiple immune modulators as shown above, the immune checkpoint LAG3 was found to be most highly expressed on both proliferating CD4⁺ Th and CD8⁺ Tc cells, and anti-LAG3 treatment most significantly enhanced their MM-specific immune responses. The Examples above further demonstrate decreased effector CD4⁺ Th cells, increased Treg and G-type MDSC, as well as upregulation of immune checkpoints on both effector/regulatory cells and patients CD138⁺ tumor cells in MM, compared to patients with MGUS, SMM or healthy individuals. Of immune modulators profiled, LAG3 expression and impact of anti-LAG3 treatment was low on G-type MDSC and Tregs, suggesting that anti-LAG3 treatment will not enhance immunosuppression conferred by these accessory cells in the BM milieu. In evaluation of XBP-1/CD138/CS-1 peptides-specific CTL with anti-MM activity, it was shown that anti-LAG3 treatment induced enhanced proliferation of both CM and EM memory CTL subsets and their functional anti-MM activities including cytotoxicity and Th1-type cytokine production. Importantly, The Examples above demonstrate that GAL-3, the ligand for LAG3, was found to be robustly expressed on CD138⁺ MM cells, and confirmed that anti-GAL-3 treatment can similarly augment immune responses against MM cells in patient BM, as well as XBP-1/CD138/CS-1 antigen-specific CTL. These studies identify and validate the potential blockade of LAG3/GAL-3 to enhance anti-tumor immune responses in MM.

Checkpoint blockade is a revolutionary cancer immunotherapy; however, a large proportion (70%-80%) of checkpoint inhibitor-treated cancer patients do not benefit due to either intrinsic or acquired resistance. See, e.g., Park RLL et al, Front Oncol 2020; 10: 258; Gremese E, et al., Clin Immunol 2020; 214: 108395; Kumar P, et al., Semin Cancer Biol 2020; 64: 29-35; Porcu M, et al. Cancers 2019; 11: 305-320. Multiple factors contribute to checkpoint blockade resistance including a lack of antigen-specific immune responses and/or impaired infiltration of effector T cells to tumor sites. The studies, as described in the Examples above were conducted to better elucidate potential mechanisms whereby immune inhibitory receptors and ligands regulate innate and adaptive immunity in MM, and specifically delineate potential mechanisms of resistance to checkpoint blockade in MM. As demonstrated, a direct beneficial impact of checkpoint inhibitor treatment was seen on T cell functional activity in BM cells from MM patients. Specifically, checkpoint inhibitor (especially anti-LAG3) treatment significantly increased T cell responses in BMMC/PBMC from MM patients compared with healthy donors.

Along with identifying the potential functional role of checkpoint inhibitors, we also examined the impact of immune agonists such as OX40 (CD134) and GITR (CD357) to activate costimulatory molecules on effector cells and thereby enhance their immune responses. In both MM patient BMMC and PBMC, immune agonist treatment enhanced immune responses and T cell proliferation. Importantly, the studies described in the Examples demonstrate higher intracellular than cell surface checkpoint expression on CD3⁺ T cells and CD138⁺ MM cells in patient BMMC suggesting that high intracellular levels of checkpoints may provide a continuous source of checkpoint molecules for translocation to the cell surface, thereby maintaining ongoing checkpoint-driven immune resistance in MM. Moreover, these studies show that treatment of MM patient BMMC with one checkpoint inhibitor (e.g., anti-PD-1 antibody) can upregulate expression of another checkpoint (e.g., LAG3), as well as expansion of regulatory and suppressor cells, in addition to effector CD3⁺ T cells. Taken together, these data identify alternative mechanisms of immune resistance induced by checkpoint inhibitors and immune agonists in MM.

Among the clinical grade checkpoint inhibitors and immune agonists evaluated in these studies, anti-PD1 treatment induced the highest level of CD4⁺ Treg expansion and upregulation of other immune checkpoints. Surprisingly, anti-LAG3 treatment induced the most robust effector T cell proliferation, while inducing the lowest level of induction of other checkpoints and Treg expansion. In addition, a higher intracellular expression of LAG3 was seen compared to PD1 in MM patient (N=5) BMMC. These findings suggest that LAG3 blockade in MM can be more effective than PD1 blockade, with a lower induction of alternative checkpoint molecules and a higher induction of effector T cell proliferation and response. These results are of particular relevance, given recent toxicity concerns observed when combining pembrolizumab (anti-PD1) with immunomodulatory drugs lenalidomide or pomalidomide or with daratumumab (anti-CD38) in recent clinical trials for relapsed MM patients. Considering the robust LAG3 expression in the tumor microenvironment and its correlation with poor prognosis in MM and other cancers, targeting the LAG3-specific inhibitory pathway may enhance anti-MM immunity and have a more favorable therapeutic index. Importantly, among the clinical grade checkpoint inhibitors and immune agonists evaluated in these studies, anti-LAG3 treatment significantly enhanced the proliferation of MM-specific effector cells and their functional activities in response to MM. Based on these results, it is proposed that anti-LAG3 can effectively overcome immunosuppression in the tumor microenvironment and be used, either alone or in combination with other immune therapies such as MM-specific vaccination (e.g., with one or more immunogenic peptides from XBP1, CS1 and CD138) and/or adoptive T-cell therapy (e.g., with XBP1 and/or CS1 and/or CD138-specific T cells, such as CTLs), to enhance generation and maintenance of antigen-specific memory CTL function against tumors. Such a strategy may achieve long-term anti-tumor immunity in MM patients. Pharmacological blockade of PD1 or PD-L1 has been at the forefront of immunotherapy for various cancers, as it reinvigorates exhausted T cells in the tumor microenvironment, thereby facilitating robust anti-tumor immune responses. However, up to 50% of patients with PD-L1 positive tumors show acquired resistance or relapse after an initial response to PD1/PD-L1 blockade, highlighting the need to target alternative pathways of inhibitory checkpoint receptor/ligand interaction to improve clinical outcomes. To address this concern and in the context of the surprising and unexpected anti-LAG3 data in MM as described in the Examples, the expression of LAG3 ligands, GAL-3 and HLA-DP/DQ/DR, on CD138⁺ MM cells in patient BMMC and MM cell lines was also evaluated. Surprisingly, it was found that blockade of GAL-3, but not HLA-DP/DQ/DR, enhanced proliferation of both CD4⁺ Th and CD8⁺ Tc cell subsets in BMMC from MM patients, independent of the cell surface or intracellular expression levels of the two respective ligands on primary CD138⁺ MM cells. Thus, checkpoint ligands expression level itself might not be the only factor, which influences effector T cell function and proliferation. The expression level and specificity/affinity between checkpoint receptor (LAG3) on the patients' T cells and the checkpoint ligands (GAL-3, HLA-DP/DQ/DR) on patients' tumor cells are a critical consideration impacting the functional sequelae of their interaction. Recently, Kundapura et al. (Kundapura, S.V., Ramagopal, U.A. Sci Rep 9, 2019: 19191) demonstrated that the CC′ loop of IgV domains of the immune checkpoint receptors, a loop which is distinct from CDRs of antibodies, plays a pivotal role in receptor: ligand affinity modulation. They proposed that a ˜5 amino acid residue long CC′ loop in a ˜120 residue protein makes a significant number of hydrophobic and polar interactions with its cognate checkpoint ligand and suggested that the CC′ loop might be a hotspot for checkpoint receptor modification that enhance their affinity for ligand interaction. In addition, we propose that the interaction between receptor and ligand can be influenced by the unique T cell receptor repertoire of each individual, resulting in variable levels or profiles of T cell or CTL functional responses and proliferation. GAL-3 is a key regulator of cell adhesion and inflammation in cancer and negatively regulates T cell function and proliferation through interaction with LAG3, especially on CD8⁺ CTL, possibly by reducing the affinity of the T-cell receptor and its internalization.

Importantly, the studies described in this disclosure demonstrate increased MM-specific CD8⁺ Tc cells expansion and selective anti-MM immune activities after anti-GAL-3 treatment of both MM patient BMMC and XBP1/CD138/CS1-specific CTL. These findings further indicate the potential role for LAG3 and/or GAL-3 inhibition, alone, and in combination with XBP1/CD138/CS1 peptide vaccination and/or adoptive T cell therapy with T cells generated against one or more of XBP1, CS1 and CD138, to augment MM-specific memory CD8⁺ CTL anti-tumor activities against MM. Further, these studies provide a basis for using the combination therapy of anti-LAG3 and/or anti-GAL3 antibodies with immunotherapeutic approaches (such as XBP1/CD138/CS1 specific peptide vaccination or cell-based therapy (e.g., ex vivo activated XBP1, CS1, and/or CD138-specific T lymphocytes therapy)) to improve patient outcome in pre-cancerous conditions (e.g., SMM or MGUS), as well as blood cancers such as MM.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a human subject with a blood cancer or a pre-cancerous blood condition, the method comprising administering to the human subject a therapeutically effective amount of one or more inhibitory agents that inhibit interaction between human Lymphocyte-activation gene 3 (LAG3) and human Galectin-3 (GAL3).

2. A method of treating a human subject with a pre-cancerous blood condition, or a blood cancer wherein pre-cancerous cells or cancer cells of these conditions express one or more of X-Box Binding Protein 1 (XBP1), CD2 Subset 1 (CS1), and CD138, the method comprising administering to the human subject a combination of
(a) a therapeutically effective amount of one or more inhibitory agents that inhibit the interaction between human LAG3 and human GAL3; with
(b) at least one of
(i) a multipeptide vaccine comprising a mixture of immunogenic peptides from one or more of XBP1, CS1, and CD138 that can induce antigen-specific T lymphocytes with anti-cancer activity;
(ii) ex vivo activated XBP1, CS1, and/or CD138-specific T lymphocytes or peripheral blood mononuclear cells (PBMCs) with anti-cancer activity; and
(iii) nanoparticles comprising a mixture of immunogenic peptides from one or more of XBP1, CS1, and CD138 that can induce antigen-specific T lymphocytes with anti-cancer activity.

3. A method for inhibiting progression from smoldering multiple myeloma (SMM) or monoclonal gammopathy of undermined significance (MGUS) to multiple myeloma (MM) in a human subject in need thereof, the method comprising administering to the human subject a combination of
(a) a therapeutically effective amount of one or more inhibitory agents that inhibit the interaction between human LAG3 and human GAL3; with
(b) at least one of
(i) a multipeptide vaccine comprising a mixture of immunogenic peptides from one or more of XBP1, CS1, and CD138 that can induce antigen-specific T lymphocytes with anti-cancer activity;
(ii) ex vivo activated XBP1, CS1, and/or CD138-specific T lymphocytes or peripheral blood mononuclear cells (PBMCs) with anti-cancer activity; and
(iii) nanoparticles comprising a mixture of immunogenic peptides from one or more of XBP1, CS1, and CD138 that can induce antigen-specific T lymphocytes with anti-cancer activity.

4. The method of any one of claims 1-3, wherein the inhibitory agent is
(a) an anti-LAG3 antibody, that specifically binds to human LAG3;
(b) an anti-GAL3 antibody, that specifically binds to human GAL3;
(c) a polypeptide comprising an extracellular domain of LAG3 that binds GAL3; or
(d) a GAL3 inhibitor.

5. The method of claim 4, wherein the inhibitory agent triggers T lymphocyte proliferation in the human subject and/or Cluster of Differentiation 107a (CD107a) degranulation and/or Th1-type cytokine production.

6. The method of claim 4, wherein the anti-LAG3 antibody and the anti-GAL3 antibody is a chimeric antibody, a humanized antibody, a bispecific antibody, or an antigen-binding fragment.

7. The method of claim 4, wherein the anti-LAG3 antibody and the anti-GAL3 antibody is an antigen-binding fragment that is an Fab, F(ab)₂, a scFv, a sc(Fv)₂, or a diabody.

8. The method of claim 4, wherein
the anti-LAG3 antibody is
(i) an anti-LAG3 antagonistic antibody (optionally, Relatlimab, Encelimab, Favezelimab, Fianlimab, Ieramilimab, or Miptenalimab);
(ii) an anti-LAG3 depleting antibody (optionally IMP731, or GSK2831781); or
(iii) a soluble LAG3 immunoglobulin (optionally Eftilagimod alpha);
(iv) a bispecific antibody that binds LAG3 and Programmed cell death protein 1 (PD1), or LAG3 and tumor necrosis factor receptor superfamily, member 4 (OX40), or LAG3 and Glucocorticoid-Induced Tumor Necrosis Factor Receptor-Related (GITR);
the GAL3 inhibitor is
(i) a small molecule inhibitor of LAG3 or GAL3; or
(ii) GB0139 (TD139), belapectin, or modified citrus pectin.

9. The method of claim 4, wherein the anti-LAG3 antibody and/or the anti-GAL3 antibody has one or more of the following functions:
(a) blocks interaction between LAG3 and GAL3;
(b) the anti-LAG3 antibody competes with GAL3 for LAG3 binding and/or the anti-GAL3 antibody competes with LAG3 for GAL3 binding;
(c) blocks LAG3 and/or GAL3 activation; or
(d) blocks LAG3 and/or GAL3 signaling.

10. The method of any one of claims 1-3, wherein the human subject is concurrently treated with one or more additional treatments, wherein the additional treatment is one or more forms of ionizing radiation and/or one or more agents selected from the group consisting of a therapeutic antibody, an immunomodulatory drug, a histone deacetylase (HDAC) inhibitor, an antineoplastic agent, a proteasome inhibitor, an antibody-drug conjugate, a nuclear export inhibitor; a corticosteroid.

11. The method of claim 10, wherein
the therapeutic antibody is selected from one or more of an anti-PD1 antibody (optionally Pembrolizumab or Nivolumab), an anti-PD-L1 antibody (optionally Durvalumab), an anti-CD38 antibody (optionally Daratumumab or Isatuximab), an anti-SLAMF7 antibody (optionally Elotuzumab), an anti-CTLA4 antibody (optionally Ipilimumab or Tremelimumab), an anti-TIM3 antibody (optionally Cobolimab), an anti-VISTA antibody (optionally SG7 or W0180), an anti-OX-40 antibody (optionally PF-04518600, or IBI101), and an anti-GITR antibody (optionally BMS-986156);
the immunomodulatory drug is selected from one or more of lenalidomide, pomalidomide, and thalidomide;
the HDAC inhibitor is citarinostat and/or panobinostat;
the antineoplastic agent is selected from one or more of cyclophosphamide, etoposide, oxorubicin, liposomal doxorubicin, melphalan, melphalan flufenamide, and bendamustine;
the proteasome inhibitor is selected from one or more of bortezomib, carfilzomib, and ixazomib;
the antibody-drug conjugate is belantamab mafodotin-blmf;
the nuclear export inhibitor is selinexor; and
the corticosteroid is dexamethasone and/or prednisone.

12. The method of claim 1 or 2, wherein the blood cancer is multiple myeloma (MM), leukemia, Non-Hodgkin lymphoma (NHL), or Waldenstrom's macroglobulinemia; and the pre-cancerous blood condition is smoldering multiple myeloma (SMM) or monoclonal gammopathy of undermined significance (MGUS).

13. The method of claim 12, wherein the MM is active MM, newly diagnosed MM, relapsed MM, or relapsed/refractory MM.

14. The method of claim 2 or 3, wherein the peptide vaccination further comprises administering an adjuvant (optionally incomplete Freund's adjuvant (IFA), Polyinosinic-polycytidylic acid, or poly-L-lysine (poly-ICLC)).

15. The method of claim 5, wherein the Th1-type cytokine is one or more of interferon-γ (IFNγ), interleukin 2 (IL-2), IL-12, IL-18, and IL-27.

16. The method of claim 2 or 3, wherein the mixture of immunogenic peptides is selected from
(a) one, two, three, or four HLA-A2-restricted peptides recited below:
(i) a peptide of 50 amino acids or less in length comprising the amino acid sequence of non-spliced XBP1 set forth in SEQ ID NO: 24 (YISPWILAV) with 0, 1, 2, 3, or 4 substitutions, wherein the non-spliced XBP1 peptide binds to HLA-A2;
(ii) a peptide of 50 amino acids or less in length and comprising the amino acid sequence of spliced XBP1 set forth in SEQ ID NO: 25 (YLFPQLISV) with 0, 1, 2, 3, or 4 substitutions, wherein the spliced XBP1 peptide binds to HLA-A2;
(iii) a peptide of 50 amino acids or less in length and comprising the amino acid sequence of CD138 peptide set forth in SEQ ID NO: 26 (GLVGLIFAV) with 0, 1, 2, 3, or 4 substitutions, wherein the CD138 peptide binds to HLA-A2; and
(iv) a peptide of 50 amino acids or less in length comprising the amino acid sequence of CS-1 set forth in SEQ ID NO: 27 (SLFVLGLFL) with 0, 1, 2, 3, or 4 substitutions, wherein the CS-1 peptide binds to HLA-A2;
or
(b) one, two, three, or four HLA-A24-restricted peptides recited below
(i) a peptide of 50 amino acids or less in length comprising the amino acid sequence of non-spliced XBP1 set forth in SEQ ID NO: 28 (ISPWILAVL) with 0, 1, 2, 3, or 4 substitutions, wherein the non-spliced XBP1 peptide binds to HLA-A24;
(ii) a peptide of 50 amino acids or less in length and comprising the amino acid sequence of spliced XBP1 set forth in SEQ ID NO: 29 (VYPEGPSSL) with 0, 1, 2, 3, or 4 substitutions, wherein the spliced XBP1 peptide binds to HLA-A24;
(iii) a peptide of 50 amino acids or less in length and comprising the amino acid sequence of CD138 set forth in SEQ ID NO: 30 (IFAVCLVGF) with 0, 1, 2, 3, or 4 substitutions, wherein the CD138 peptide binds to HLA-A24; and
(iv) a peptide of 50 amino acids or less in length comprising the amino acid sequence of CS-1 set forth in SEQ ID NO: 32 (LFVLGLFLW) with 0, 1, 2, 3, or 4 substitutions, wherein the CS-1 peptide binds to HLA-A24.

17. The method of claim 2 or 3, wherein the mixture of immunogenic peptides is selected from
(a) one, two, three, or four of HLA-A2-restricted peptides recited below:
(i) a peptide comprising the amino acid sequence of non-spliced XBP1 set forth in SEQ ID NO: 24 (YISPWILAV);
(ii) a peptide comprising the amino acid sequence of spliced XBP1 set forth in SEQ ID NO: 25 (YLFPQLISV);
(iii) a peptide comprising the amino acid sequence of CD138 set forth in SEQ ID NO: 26 (GLVGLIFAV); and
(iv) a peptide of comprising the amino acid sequence of CS-1 set forth in SEQ ID NO: 27 (SLFVLGLFL); or
(b) one, two, three, or four of HLA-A24-restricted peptides recited below:
(i) a peptide comprising the amino acid sequence of non-spliced XBP1 set forth in SEQ ID NO: 28 (ISPWILAVL);
(ii) a peptide comprising the amino acid sequence of spliced XBP1 set forth in SEQ ID NO: 29 (VYPEGPSSL);
(iii) a peptide comprising the amino acid sequence of CD138 set forth in SEQ ID NO: 30 (IFAVCLVGF); and
(iv) a peptide comprising the amino acid sequence of CS-1 set forth in SEQ ID NO: 31 (LFVLGLFLW).

18. The method of claim 2 or 3, wherein the mixture of immunogenic peptides is selected from (a) one, two, three, or four of HLA-A2-restricted peptides recited below:
(i) a peptide consisting of the amino acid sequence of non-spliced XBP1 set forth in SEQ ID NO: 24 (YISPWILAV);
(ii) a peptide consisting of the amino acid sequence of spliced XBP1 set forth in SEQ ID NO: 25 (YLFPQLISV);
(iii) a peptide consisting of the amino acid sequence of CD138 set forth in SEQ ID NO: 26 (GLVGLIFAV); and
(iv) a peptide of consisting of the amino acid sequence of CS-1 set forth in SEQ ID NO: 27 (SLFVLGLFL); or
(b) one, two, three, or four of HLA-A24-restricted peptides recited below:
(i) a peptide consisting of the amino acid sequence of non-spliced XBP1 set forth in SEQ ID NO: 28 (ISPWILAVL);
(ii) a peptide consisting of the amino acid sequence of spliced XBP1 set forth in SEQ ID NO: 29 (VYPEGPSSL);
(iii) a peptide consisting of the amino acid sequence of CD138 set forth in SEQ ID NO: 30 (IFAVCLVGF); and
(iv) a peptide consisting of the amino acid sequence of CS-1 set forth in SEQ ID NO: 31 (LFVLGLFLW).

19. The method of claim 2 or 3, wherein the ex vivo activated T lymphocytes are generated by the following steps:
(a) providing or isolating T lymphocytes and antigen presenting cells from the human subject or an HLA-matched donor;
(b) contacting the antigen presenting cells with a multipeptide vaccine comprising three or more of a non-spliced XBP1 peptide, a spliced XBP1 peptide, a CD138 peptide, and a CS-1 peptide; and
(c) contacting the T lymphocytes with the antigen presenting cells from step (b) to generate ex vivo activated T lymphocytes.

20. The method of claim 2 or 3, wherein the ex vivo activated PBMCs are generated by the following steps:
(a) providing or isolating PBMCs from the human subject or an HLA-matched donor;
(b) contacting the PBMCs with a mixture of immunogenic peptides comprising three or more of a non-spliced XBP1 peptide, a spliced XBP1 peptide, a CD138 peptide, and a CS-1 peptide; and
(c) generating ex vivo activated PBMCs.

21. The method of claim 2 or 3, wherein the components of (a) and (b) of the combination are administered simultaneously, sequentially or, alternately.

22. The method of claim 2 or 3, wherein the components of (a) and (b) of the combination are administered multiple times during treatment.

23. The method of claim 2 or 3, wherein the components of (a) and (b) of the combination are administered intravenously, intra-arterially, subcutaneously, intramuscularly, intraperitoneally, transdermally, orally, sublingually, intranasally, or transmucosally to the subject.

24. The method of claim 10, wherein the method increases at least one of the following parameters in the human subject relative to a control population treated with the one or more additional treatments alone:
(a) objective response rate (ORR);
(b) time to next treatment (TTNT);
(c) overall survival (OS);
(d) progression free survival (PFS); and
(e) the chance of achieving a negative minimal residual disease (MRD).

25. The method of claim 1 or 2, wherein the human subject with the pre-cancerous blood condition does not develop MM.

26. A method for increasing T lymphocyte responses in a tumor microenvironment while reducing immunosuppression in a human subject in need thereof, the method comprising administering a combination of
(a) a therapeutically effective amount of one or more inhibitory agents that inhibit the interaction between human LAG3 and human GAL3; with
(b) a therapeutically effective amount of one or more of an anti-PD1 antibody, an anti-OX40 antibody, and an anti-GITR antibody.

27. The method of claim 26, wherein the inhibitory agent is an anti-LAG3 antibody or an anti-GAL3 antibody.

28. The method of claim 27, wherein the anti-LAG3 antibody is Relatlimab and the anti-PD1 antibody is Pembrolizumab or Nivolumab.

29. A pharmaceutical composition comprising (a) any one or a mixture of two or more immunogenic peptides, wherein the immunogenic peptides comprise the amino acid sequence of a non-spliced XBP1 peptide, a spliced XBP1 peptide, a CD 138 peptide, and a CS1 peptide; (b) a LAG3 inhibitory agent and/or a GAL3 inhibitory agent; and (c) a pharmaceutically acceptable carrier.

30. A combination comprising (a) a multipeptide vaccine comprising at least three immunogenic peptides, wherein the immunogenic peptides comprise the amino acid sequence of a non-spliced XBP1 peptide, a spliced XBP1 peptide, a CD 138 peptide, and a CS-1 peptide; and (b) a composition comprising a LAG3 inhibitory agent and/or a GAL3 inhibitory agent.

31. A combination comprising (a) means for targeting HLA-A2+ or HLA-A24+ pre-cancerous or cancerous cells that express one or more of XBP1, CS1, and CD138, and (b) a LAG3 inhibitory agent and/or a GAL3 inhibitory agent.

32. A combination comprising (a) anti-myeloma-specific T lymphocytes targeting XBP1, CD138 and CS1-expressing cells from SMM, MGUS, or MM patients and (b) means for specifically binding human LAG3 and/or means for specifically binding human GAL3.

33. A combination comprising (a) anti-myeloma-specific PBMCs targeting XBP1, CD138 and CS1-expressing cells from SMM, MGUS, or MM patients and (b) means for specifically binding human LAG3 and/or means for specifically binding human GAL3.

34. A combination comprising (a) nanoparticles comprising a mixture of immunogenic peptides from one or more of XBP1, CS1, and CD138 and (b) means for specifically binding human LAG3 and/or means for specifically binding human GAL3.

35. The combination of any one of claims 30-34, wherein (a) and (b) are formulated for administration to a human subject in need thereof, simultaneously, sequentially or, alternately.

36. A kit comprising (a) a first composition comprising at least three of a non-spliced XBP1 peptide, a spliced XBP1 peptide, a CD138 peptide and a CS-1 peptide, (b) a second composition comprising an anti-LAG3 antibody and/or an anti-GAL3 antibody, and optionally, (c) instructions for administering the first and second compositions to a subject.

37. The kit of claim 36, further comprising one or more additional therapeutic agents.


SEQUENCE LISTING (38 sequences)

SEQ ID NO: 1 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 360 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITGQPQVGKE

SEQ ID NO: 3 - protein, 5 aa, Artificial Sequence
  Description of Artificial Sequence Synthetic peptide
DYYWN

SEQ ID NO: 4 - protein, 16 aa, Artificial Sequence
  Description of Artificial Sequence Synthetic peptide
EINHRGSTNSNPSLKS

SEQ ID NO: 5 - protein, 12 aa, Artificial Sequence
  Description of Artificial Sequence Synthetic peptide
GYSDYEYNWFDP

SEQ ID NO: 6 - protein, 11 aa, Artificial Sequence
  Description of Artificial Sequence Synthetic peptide
RASQSISSYLA

SEQ ID NO: 7 - protein, 7 aa, Artificial Sequence
  Description of Artificial Sequence Synthetic peptide
DASNRAT

SEQ ID NO: 8 - protein, 9 aa, Artificial Sequence
  Description of Artificial Sequence Synthetic peptide
QQRSNWPLT

SEQ ID NO: 9 - protein, 120 aa, Artificial Sequence
  Description of Artificial Sequence Synthetic polypeptide
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 10 - protein, 107 aa, Artificial Sequence
  Description of Artificial Sequence Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 11 - protein, 447 aa, Artificial Sequence
  Description of Artificial Sequence Synthetic polypeptide
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, Artificial Sequence
  Description of Artificial Sequence Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 250 aa, Homo sapiens [sequence omitted]

SEQ ID NO: 14 - protein, 16 aa, Artificial Sequence
  Description of Artificial Sequence Synthetic peptide
RSSKSLLYKDGKTYLN

SEQ ID NO: 15 - protein, 7 aa, Artificial Sequence
  Description of Artificial Sequence Synthetic peptide
LMSTHAS

SEQ ID NO: 16 - protein, 9 aa, Artificial Sequence
  Description of Artificial Sequence Synthetic peptide
QQLVDYPLT

SEQ ID NO: 17 - protein, 7 aa, Artificial Sequence
  Description of Artificial Sequence Synthetic peptide
GYTFTNY

SEQ ID NO: 18 - protein, 6 aa, Artificial Sequence
  Description of Artificial Sequence Synthetic peptide
NTNTGE

SEQ ID NO: 19 - protein, 7 aa, Artificial Sequence
  Description of Artificial Sequence Synthetic peptide
YDNFFAY

SEQ ID NO: 20 - protein, 115 aa, Artificial Sequence
  Description of Artificial Sequence Synthetic polypeptide
QVQLVQSGSELKKPGASVKVSCKASGYTFTNYGMNWVRQAPGQGLKWMGWINTNTGEPTYAQEFTGRFVFSLDTSVSTAYLQISSLKAEDTAVYFCAPYDNFFAYWGQGTTVTVS

SEQ ID NO: 21 - protein, 112 aa, Artificial Sequence
  Description of Artificial Sequence Synthetic polypeptide
DIVLTQSPLSLPVTPGEPASISCRSSKSLLYKDGKTYLNWFLQKPGQSPQLLIYLMSTHASGVPDRFSGSGSGTDFTLKISRVEAEDVGVYYCQQLVDYPLTFGGGTKLEIK

SEQ ID NO: 22 - protein, 442 aa, Artificial Sequence
  Description of Artificial Sequence Synthetic polypeptide
QVQLVQSGSELKKPGASVKVSCKASGYTFTNYGMNWVRQAPGQGLKWMGWINTNTGEPTYAQEFTGRFVFSLDTSVSTAYLQISSLKAEDTAVYFCAPYDNFFAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 219 aa, Artificial Sequence
  Description of Artificial Sequence Synthetic polypeptide
DIVLTQSPLSLPVTPGEPASISCRSSKSLLYKDGKTYLNWFLQKPGQSPQLLIYLMSTHASGVPDRFSGSGSGTDFTLKISRVEAEDVGVYYCQQLVDYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 9 aa, Homo sapiens
YISPWILAV

SEQ ID NO: 25 - protein, 9 aa, Homo sapiens
YLFPQLISV

SEQ ID NO: 26 - protein, 9 aa, Homo sapiens
GLVGLIFAV

SEQ ID NO: 27 - protein, 9 aa, Homo sapiens
SLFVLGLFL

SEQ ID NO: 28 - protein, 9 aa, Homo sapiens
ISPWILAVL

SEQ ID NO: 29 - protein, 9 aa, Homo sapiens
VYPEGPSSL

SEQ ID NO: 30 - protein, 9 aa, Homo sapiens
IFAVCLVGF

SEQ ID NO: 31 - protein, 9 aa, Homo sapiens
LFVLGLFLW

SEQ ID NO: 32 - protein, 9 aa, Homo sapiens
LFVLGLFLW

SEQ ID NO: 33 - protein, 376 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 34 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 35 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 36 - protein, 447 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 200 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEV

SEQ ID NO: 38 - protein, 261 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAALRLRAPLQQVQAQLSPLQNISPWILAVLTLQIQSLISCWAFWTTWTQSCSSNALPQSLPAWRSSQRSTQKDPVPYQPPFLCQWGRHQPSWKPLMN